(19) United States
(12) Patent Application Publication
(10) Pub. No.: US 2019/0185541 A1
(43) Pub. Date: Jun. 20, 2019

(54) METHODS FOR SELECTING AND TREATING PATIENTS WITH A TRAIL-BASED THERAPEUTIC OR DEATH RECEPTOR AGONIST

(71) Applicant: Merrimack Pharmaceuticals, Inc., Cambridge, MA (US)

(72) Inventors: Tamara DAKE, Sharon, MA (US); Sara GHASSEMIFAR, Medford, MA (US); Yasmin HASHAMBHOY-RAMSAY, Watertown, MA (US); Diana Hung-yi Chai MARCANTONIO, Concord, MA (US); Eric M. TAM, Cambridge, MA (US); Haluk YUZUGULLU, Newton, MA (US)

(21) Appl. No.: 16/305,289

(22) PCT Filed: Jun. 13, 2017
(86) PCT No.: PCT/US2017/037259

§ 371 Date: Nov. 28, 2018

Related U.S. Application Data
(60) Provisional application No. 62/458,824, filed on Feb. 14, 2017.
(60) Provisional application No. 62/349,497, filed on Jun. 13, 2016.

(51) Int. Cl. C07K 14/705; A61P 35/00; A61K 47/60

(52) U.S. Cl. CPC C07K 14/70575; A61P 35/00; A61K 38/00; C07K 2319/30; A61K 47/60

(57) ABSTRACT

Provided herein are methods of identifying cancer patients (e.g., colorectal cancer cancers) who will benefit from treatment with a TRAIL-based therapeutic or death receptor agonist based on their levels of DR4 and cIAP1. Also provided are methods of treating a patient diagnosed with cancer (e.g., colorectal cancer) based on their levels of DR4 and cIAP1.

### RELATED APPLICATIONS

This application claims priority to U.S. Provisional Application No. 62/349,497, filed Jun. 13, 2016 and U.S. Provisional Application No. 62/458,824, filed Feb. 14, 2017. The contents of the aforementioned application is hereby incorporated by reference.

### BACKGROUND

Apo2L/TRAIL (tumor necrosis factor (TNF)-related apoptosis-inducing ligand, also known as CD253) is a member of the TNF family that binds and activates the death receptors (specifically DR4 and DR5). TRAIL also binds non-signaling decoy receptors, DcR1, DcR2, and osteoprotegerin (OPG). TRAIL naturally occurs as a type 2 transmembrane protein, with an extracellular domain that can be cleaved to release a soluble trimeric protein. Clustering of the receptor complex, e.g., as mediated by the trimeric structure of TRAIL, is necessary for efficient signaling and induction of apoptosis by death receptors. Additionally, higher order oligomerization of receptor complexes can amplify signaling, resulting in greater induction of apoptosis.

Over the years, there has been interest in TRAIL and its potential in oncology due to its ability to induce apoptosis preferentially in cancer cells; however, clinical success of recombinant human TRAIL (rhTRAIL) and other death receptor agonists have been limited. Two possible factors contributing to this lack of clinical success include sub-optimal agonist design and intrinsic resistance in patient tumors.

The present disclosure addresses this need and provides additional advantages.

### SUMMARY

Provided herein are methods of identifying cancer patients (e.g., colorectal cancer patients) who will benefit from treatment with a TRAIL-based therapeutic of a death receptor agonist based on their particular levels of DR4 and/or cIAP1 expression. Also provided are methods of treating a cancer patient who has been determined to have particular levels of one or more biomarkers (DR4 and/or cIAP1) by administering a TRAIL-based therapeutic or death receptor agonist.

In another aspect, a two gene predictive biomarker allows identification of patient responders. This latter aspect is based on the observation that death receptor 4 (DR4) and cellular inhibitor of apoptosis 1 (cIAP1) are important nodes in the TRAIL cell signaling pathway. It has been discovered that when the levels of death receptor 4 (DR4) are high and the levels of cIAP1 are low, treatment with a TRAIL antagonist or death receptor (DR4 or DR5) agonist is very effective in inducing cell death, and thus a reduction in tumor size.

In one embodiment, levels of DR4 in the patient are high enough and levels of cIAP1 in the patient are low enough that treating the patient results in greater than 50% cancer cell death.

In another embodiment, the treatment results in greater than 60% cancer cell death, greater than 65% cancer cell death, greater than 70% cancer cell death, greater than 75% cancer cell death, greater than 80% cancer cell death, greater than 85% cancer cell death, greater than 90% cancer cell death, greater than 95% cancer cell death, or 100% cancer cell death. In one embodiment, methods of treating a patient diagnosed with cancer (e.g., colorectal cancer) having a tumor with high levels of DR4 and low levels of cIAP1 are provided, the method comprising administering a therapeutically effective amount of a TRAIL-based therapeutic or a death receptor agonist.

In another aspect, a one gene predictive biomarker allows identification of patient responders. For example, in one embodiment, the levels of DR4 are high enough that treating the patient results in greater than 50% cancer cell death. In another embodiment, the treatment results in greater than 60% cancer cell death, greater than 70% cancer cell death, greater than 80% cancer cell death, great than 90% cancer cell death, or 100% cancer cell death.

In one embodiment, methods of treating a patient diagnosed with cancer (e.g., colorectal cancer) having a tumor with high levels of DR4 and/or cIAP1 are provided, the method comprising administering a therapeutically effective amount of a death receptor agonist. In one embodiment, the death receptor agonist is a DR4 agonist. In another embodiment, the death receptor agonist is a DRS agonist. In another embodiment, the death receptor agonist is a dual DR4/5 agonist.

In another embodiment, methods of treating a patient diagnosed with cancer (e.g., colorectal cancer) having a tumor with high levels of DR4 and/or cIAP1 are provided, the method comprising administering a therapeutically effective amount of a TRAIL-based therapeutic. In various embodiments, such TRAIL-based therapeutics may be cross-linked, PEGylated, or recombinant human trail (rhTRAIL). In one embodiment, the TRAIL-based therapeutic is a TRAIL polypeptide. In another embodiment, the TRAIL polypeptide is a TRAIL monomer, dimer, or trimer. In another embodiment, the TRAIL polypeptide comprises at least one stabilizing mutation. In another embodiment, the TRAIL polypeptide is an Fc-TRAIL fusion polypeptide. In another embodiment, the TRAIL polypeptide is an Fc-TRAIL fusion polypeptide selected from the group consisting of “Fc-T148”, “Fc-T151”, “Fc-T153”, “Fc-T182”, “Fc-T183”, “Fc-T186”, “Fc-T191”, “Fc-T196”, “Fc-T202”, “Fc-T203”, “Fc-T204”, “Fc-T205”, “Fc-T206”, “Fc-T207”, “Fc-T208”, “Fc-T209”, “Fc-T210”, and “Fc-T211” (SEQ ID NO: 22-39, respectively). In another embodiment, the Fc-TRAIL fusion polypeptide is Fc-T191 (SEQ ID NO: 28).

Levels of DR4 and/or cIAP1 can be measured by any suitable technique, including measurement of RNA or protein. In one embodiment, RNA levels of DR4 and/or cIAP1 are measured by RT-PCR, quantitative fluorogenic RT-PCR, or RNA-ISH. In another embodiment, protein levels of DR4 and/or cIAP1 are measured by immunohistochemistry (IHC), enzyme-linked immunosorbent assay (ELISA), or western blot.

In one embodiment, the patient sample is determined to have a DR4 IHC score of 1 or higher, 2 or higher, or 3 or higher. In another embodiment, the patient sample is determined to have a DR4 RNA-ISH score of 1+ or higher, 2+ or higher, or 3+ or higher. In another embodiment, the patient sample is determined to have a DR4 RT-PCR score of greater than or equal to −5.

In another embodiment, the patient sample is determined to have a cIAP1 IHC score of 0, 1 or lower, or 2 or lower. In another embodiment, the patient sample is determined to have a cIAP1 RNA-ISH score of 0, 1+ or lower, or 2+ or lower. In another embodiment, the patient sample is determined to have a cIAP1 RT-PCR score of less than or equal to 5.

In another embodiment, the biomarkers are measured as a ratio of DR4 to cIAP1 (i.e., DR4/cIAP1). In one embodiment, the relative DR4/cIAP1 ratio is at least about 0.5, at least about 0.6, at least about 0.7, at least about 0.8, at least about 0.9, at least about 1.0, at least about 1.1, at least about 1.2, at least about 1.3, at least about 1.4, at least about 1.5, at least about 1.6, at least about 1.7, at least about 1.8, at least about 1.9, or at least about 2.0. In another embodiment, the DR4/cIAP1 ratio is at least about 0.5. In another embodiment, the DR4/cIAP1 ratio is at least about 0.7.

In another embodiment, methods of treating a patient having a cancer (e.g., colorectal cancer) that has been determined to have a DR4/cIAP1 ratio of at least about 0.5 are provided, the method comprising administering a therapeutically effective amount of a TRAIL-based therapeutic (e.g., a TRAIL-polypeptide or Fc-TRAIL fusion polypeptide, such as Fc-T191 (SEQ ID NO:28), or a death receptor agonist (e.g., a DR4, DRS, or DR4/DR5 agonist).

In another embodiment, methods of treating a patient having a colorectal cancer that has been determined to have a DR4/cIAP1 ratio of at least about 0.5 are provided, wherein the method comprises administering a therapeutically effective amount of a Fc-TRAIL fusion polypeptide comprising SEQ ID NO:28.

In another embodiment, the method comprises (1) determining the DR4/cIAP1 ratio of the cancer and (2) administering the TRAIL-based therapeutic or death receptor agonist if the DR4/cIAP1 ratio is about 0.5 or greater.

In another embodiment, the treatment methods described herein include administration of TRAIL-based therapeutic or a death receptor agonist as a monotherapy. In another aspect, methods for selecting therapy for, or for providing treatment to, a patient having a cancer (e.g. colorectal cancer) are provided, the method comprising determining a DR4/cIAP1 ratio from at least one cancer cell sample from the patient, wherein if the ratio is at least about 0.5, then 1) selecting the patient for treatment with, and/or 2) administering to the patient, an effective amount of a TRAIL-based therapeutic or death receptor agonist. In one embodiment, the DR4/cIAP1 ratio is at least about 0.5, at least about 0.6, at least about 0.7, at least about 0.8, at least about 0.9, at least about 1.0, at least about 1.1, at least about 1.2, at least about 1.3, at least about 1.4, at least about 1.5, at least about 1.6, at least about 1.7, at least about 1.8, at least about 1.9, or at least about 2.0. In a particular embodiment, the DR4/cIAP1 ratio is at least about 0.5. In another particular embodiment, the DR4/cIAP1 ratio is at least about 0.7.

In another aspect, methods of identifying cancer patients (e.g., colorectal cancer patients) who will benefit from treatment with a TRAIL-based therapeutic of a death receptor agonist based on their particular levels of DR4 and/or cIAP1 expression are provided, wherein the identification is based only on levels of DR4 and/cIAP1. For example, the method does not involve measuring levels of one or more of the following biomarkers: APAF1, CASP3, CASP6, CASP7, CASP8, CASP9, CASP10, FADD, SPTAN1, BCL2, GAS2, BID, LMNA, CFLAR, MAP3K14, BIRC3, CHUK, NFKB1, NFKBIA, TNFRSF10B, TNFSF25, TNFSF10, TNFSF12, CYCS, DFFA, DFFB, RELA, TRADD, RIPK1, TRAF2, and XIAP. In another embodiment, the method includes measuring levels of DR4 and/cIAP1 and one or more additional biomarkers, wherein the one or more additional biomarkers is not APAF1, CASP3, CASP6, CASP7, CASP8, CASP9, CASP10, FADD, SPTAN1, BCL2, GAS2, BID, LMNA, CFLAR, MAP3K14, BIRC3, CHUK, NFKB1, NFKBIA, TNFRSF10B, TNFSF25, TNFSF10, TNFSF12, CYCS, DFFA, DFFB, RELA, TRADD, RIPK1, TRAF2, and/or XIAP. In another aspect, methods of treating a cancer patient who has been determined to have particular levels of one or more biomarkers (DR4 and/or cIAP1) by administering a TRAIL-based therapeutic or death receptor agonist are provided, wherein the decision to treat a patient is based only on levels of Dr4 and/or cIAP1. For example, the patient's levels of one or more of the following biomarkers have not been assessed as part of the method: APAF1, CASP3, CASP6, CASP7, CASP8, CASP9, CASP10, FADD, SPTAN1, BCL2, GAS2, BID, LMNA, CFLAR, MAP3K14, BIRC3, CHUK, NFKB1, NFKBIA, TNFRSF10B, TNFSF25, TNFSF10, TNFSF12, CYCS, DFFA, DFFB, RELA, TRADD, RIPK1, TRAF2, and XIAP. In another embodiment, the decision to treat a patient is based on levels of Dr4 and/or cIAP1 and one or more additional biomarkers, wherein the one or more additional biomarkers is not APAF1, CASP3, CASP6, CASP7, CASP8, CASP9, CASP10, FADD, SPTAN1, BCL2, GAS2, BID, LMNA, CFLAR, MAP3K14, BIRC3, CHUK, NFKB1, NFKBIA, TNFRSF10B, TNFSF25, TNFSF10, TNFSF12, CYCS, DFFA, DFFB, RELA, TRADD, RIPK1, TRAF2, and/or XIAP.

### BRIEF DESCRIPTION OF THE DRAWINGS

FIG. 1A is a graph showing logic model scores vs. cell viability from colorectal cancer (CRC) cells exposed to cross-linked TRAIL. A logic model was created using normalized DR4 and cIAP1 gene expression levels and cell viability data from 27 CRC samples exposed to cross-linked TRAIL (training dataset). The logic model score is equal to DR4*(1-cIAP1). Logic model scores correlate with cell viability with a Pearson correlation coefficient of −0.69 (p<0.0001). Each dot represents one cell line.

FIG. 1B is a graph showing the fit logic model (see FIG. 1A) vs. cell viability from CRC cells exposed to rhTRAIL. The DR4*(1-cIAP1) model correlates with the maximum inhibition of cell viability in 11 CRC cell lines in response to rhTRAIL. Each dot represents one cell line. A regression model (red line) is fit to predict cell viability in response to 10 nM rhTRAIL as a function of the logic model score. Regression was performed using data from 10 out of the 11 cell lines. MDST8 (logic model score=0.14 and cell viability=6%) was excluded from the fit to create a predictive model because it is clearly an outlier. The regression model is: percent of viable cells remaining=86.7−122.0*(logic model score).

FIG. 1C is a graph showing the comparison between predicted and actual cell viability in response to rhTRAIL in 9 CRC cell lines. The regression model described in FIG. 1B is used to predict cell viability in response to 10 nM rhTRAIL in 9 new CRC cell lines (test set). Red circles show the model predictions. Box plots show the actual cell viability in response to rhTRAIL (4 replicates/cell line). Using 50% as a threshold for response, the model is able to accurately predict 8 out of 9 responder cell lines.

FIG. 2A is a histogram of normalized DR4 levels in normal tissues and tumor of colorectal cancer patients.

FIG. 2B is a histogram of normalized cIAP1 levels in normal tissues and tumor of colorectal cancer patients.

FIG. 2C is a histogram of biomarker logic model scores of tumor tissues of colorectal cancer patients.

FIG. 2D is a histogram of biomarker logic model scores of normal tissues of colorectal cancer patients.

FIG. 3A is a graph showing cell viability of colorectal cancer cell lines following treatment with 10 nM recombinant TRAIL.

FIG. 3B is a graph showing the ratio of DR4/cIAP1 mRNA expression in colorectal cancer cell lines, which are sensitive or resistant to TRAIL.

FIG. 3C is a scatter plot showing the relationship between TRAIL-induced growth inhibition and relative DR4/cIAP1 mRNA expression in various colorectal cancer cell lines.

FIG. 4 is a graph showing growth inhibition of colorectal cancer cell lines by rhTRAIL and Fc-T191. The anti-tumor activities were assessed at concentrations up to 10 nM for rhTRAIL and Fc-T191 using CellTiter-Glo luminescence assays. Each dot represents one cell line, and standard deviation is shown for each treatment (7 replicates for rhTRAIL, 3 replicates for Fc-T191, correlation coefficient=0.92).

FIGS. 5A-5C are graphs showing the effect of Fc-T191 on tumor volume using Colo205, RKO, and KM12 cells. Arrows indicate the day when treatment started.

FIGS. 6A-6C show that DR4 knockdown is sufficient to render LIM1215 colorectal cancer cells resistant to TRAIL. FIG. 6A is a graph showing cell viability of colorectal cancer cells lines in the presence of 30 nM Fc-T191. FIG. 6B is a western blot showing siRNA knockdown of DR4 and/or DRS in LIM1215 cells. FIG. 6C is a graph showing growth inhibition of LIM1215 cells transfected with siRNA against DR4 and/or DRS in the presence of increasing concentrations of Fc-T191.

FIGS. 7A-7C show that DR4 knockdown is sufficient to render HCT116 colorectal cancer cells resistant to TRAIL. FIG. 7A is a graph showing cell viability of colorectal cancer cells lines in the presence of 30 nM Fc-T191. FIG. 7B is a western blot showing siRNA knockdown of DR4 and/or DRS in LIM1215 cells. FIG. 7C is a graph showing growth inhibition of LIM1215 cells transfected with siRNA against Dr4 and/or DRS in the presence of increasing concentrations of Fc-T191.

FIGS. 8A-8B are graphs showing response of colorectal cancer cells to Fc-T191 in a patient-derived xenograft (PDX) mouse model. Arrows indicate the day when treatment started.

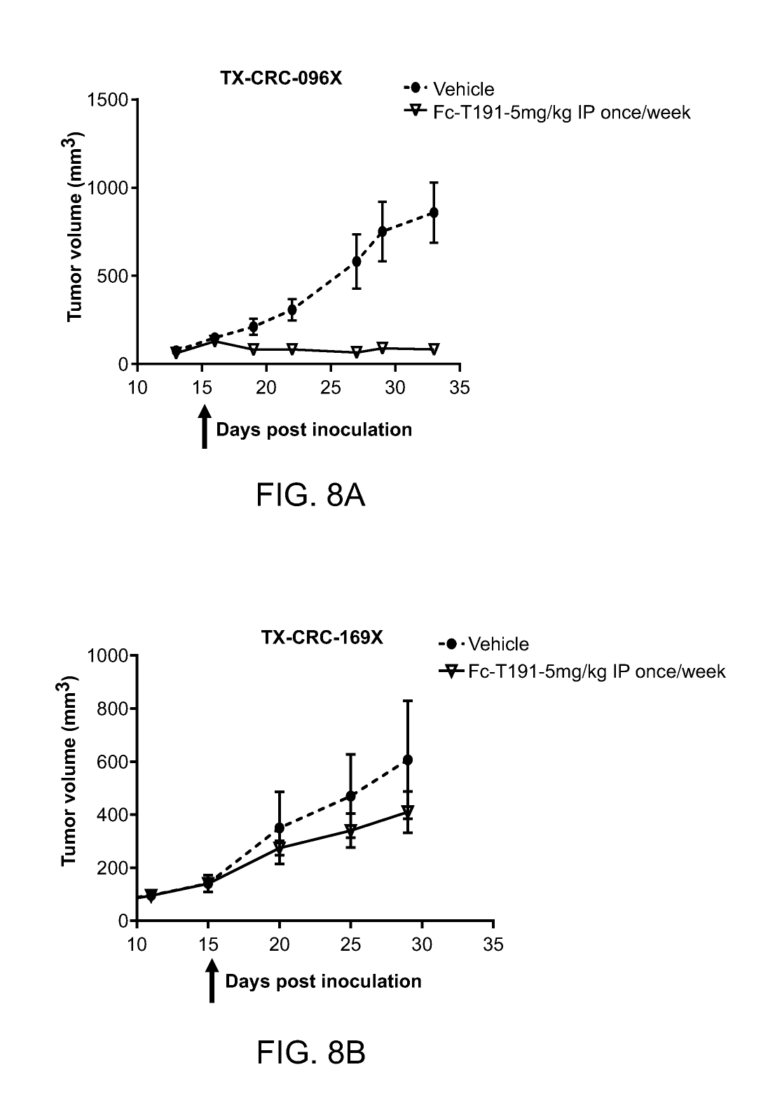

FIG. 9 is a graph showing DR4/cIAP1 ratio correlates with TRAIL response in colorectal cancer PDXs.

FIG. 10 shows the relative mRNA and protein expression of DR4 and cIAP1 in colorectal cancer PDXs.

### DETAILED DESCRIPTION

Provided herein are methods for identifying cancer patients (e.g., colorectal cancer) who will respond to treatment with a TRAIL-based therapeutic (e.g., rhTRAIL or TRAIL polypeptide) or a death receptor agonist.

### Definitions

For convenience, the meaning of certain terms and phrases used in the specification, examples, and claims, are provided below.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a”, “an”, and “the” include plural referents unless the context clearly dictates otherwise. The use of “or” or “and” means “and/or” unless stated otherwise. Furthermore, use of the term “including” as well as other forms, such as “include”, “includes”, and “included”, is not limiting.

The term “about” as used herein when referring to a measurable value such as an amount, a temporal duration and the like, encompasses variations of up to ±10% from the specified value. Unless otherwise indicated, all numbers expressing quantities of ingredients, properties such as molecular weight, reaction conditions, etc., used herein are to be understood as being modified by the term “about”.

As used herein, the term “subject” or “patient” is a human patient (e.g., a patient having a cancer, such as colorectal cancer).

The terms “treat,” “treating,” and “treatment,” as used herein, refer to therapeutic or preventative measures described herein. The methods of “treatment” employ administration to a subject the composition disclosed herein in order to prevent, cure, delay, reduce the severity of, or ameliorate one or more symptoms of the disease or disorder or recurring disease or disorder, or to prolong the survival of a subject beyond that expected in the absence of such treatment.

As used herein, “antineoplastic agent” or “anti-cancer agent” refers to agents that have the functional property of inhibiting the development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “TRAIL” (also referred to as “Apo2L/TRAIL,” “TNF-related apoptosis-inducing ligand” and “CD253”) refers to a member of the TNF family that binds and activates death receptors (specifically DR4 and DR5). The human TRAIL amino acid sequence (1-281) (NP_003801.1) is:

(SEQ ID NO: 1)
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG.

TRAIL also binds non-signaling decoy receptors, DcR1, DcR2, and osteoprotegrin (OPG, also known as osteoclastogenesis inhibitory factor (OCIF)). TRAIL naturally occurs as a type 2 transmembrane protein, with an extracellular domain that can be cleaved to release a soluble trimeric protein. Clustering of the receptor complex, e.g., as mediated by the trimeric structure of TRAIL, is necessary for efficient signaling and induction of apoptosis by the death receptors. Additionally, higher order oligomerization of receptor complexes can amplify signaling, resulting in greater induction of apoptosis.

“Peptide” or “polypeptide” refers to any peptide comprising two or more amino acids joined by peptide bonds or modified peptide bonds (e.g., peptide isosteres). Peptides can contain amino acids other than the 20 naturally occurring nucleic acid encoded amino acids, and include amino acid sequences modified either by natural processes, such as post-translational processing, or by chemical modification techniques which are well known in the art. Modifications can occur anywhere in a peptide, including the peptide backbone, the amino acid side-chains and the amino or carboxyl termini. It will be appreciated that the same type of modification can be present in the same or varying degrees at several sites in a given peptide. Also, a given polypeptide can contain many types of modifications. Polypeptides can be branched as a result of ubiquitination, and they can be cyclic, with or without branching. Cyclic, branched and branched cyclic polypeptides can result from natural posttranslational processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “variant” as used herein is defined as a modified or altered form of a wildtype sequence, e.g. where one or more amino acids may be replaced by other amino acid(s) or non-amino acid(s) which do not substantially affect function. In some embodiments, the variant may contain an altered side chain for at least one amino acid residue.

The term “inhibit” or “inhibition” means to reduce by a measurable amount.

“Inhibitors” and “antagonists,” or “activators” and “agonists,” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a compound that reduces, blocks, or inactivates a constitutive activity.

An “agonist” is a compound that interacts with a target to cause or promote an increase in the activation of the target (e.g., a polypeptide which agonizes (promotes) TRAIL signaling).

An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

One of ordinary skill in the art will appreciate that starting materials, biological and chemical materials, biological and chemical reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this disclosure.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although aspects of the present invention have been specifically disclosed by various embodiments which may include preferred embodiments, exemplary embodiments and optional features, modifications and variations of the concepts herein disclosed may be resorted to by those skilled in the art. Such modifications and variations are considered to be within the scope of embodiments of the invention as described and as may be defined by the appended claims.

### A. Biomarkers

The methods described herein involve treating a patient who has been determined to have particular levels of one or more particular biomarkers by administering a TRAIL-based therapeutic or death receptor agonist.

As used herein, “DR4” (also referred to as “TNFRSF10A,” “APO2,” “CD261,” “TRAILR-1,” and “TNF receptor superfamily member 10a”) refers to a member of the TNF receptor superfamily. The terms DR4 and TNFRSF10A are used interchangeably throughout the specification. DR4 is a membrane-bound cell surface receptor that that binds TRAIL and mediates apoptosis. The human DR4 (TNFRSF10A) amino acid sequence (1-468) (NP_003835.3) is:

(SEQ ID NO: 2)
MAPPPARVHLGAFLAVTPNPGSAASGTEAAAATPSKVWGSSAGRIEPRGG
GRGALPTSMGQHGPSARARAGRAPGPRPAREASPRLRVHKTFKFVVVGVL
LQVVPSSAATIKLHDQSIGTQQWEHSPLGELCPPGSHRSEHPGACNRCTE
GVGYTNASNNLFACLPCTACKSDEEERSPCTTTRNTACQCKPGTFRNDNS
AEMCRKCSRGCPRGMVKVKDCTPWSDIECVHKESGNGHNIWVILVVTLVV
PLLLVAVLIVCCCIGSGCGGDPKCMDRVCFWRLGLLRGPGAEDNAHNEIL
SNADSLSTFVSEQQMESQEPADLTGVTVQSPGEAQCLLGPAEAEGSQRRR
LLVPANGADPTETLMLFFDKFANIVPFDSWDQLMRQLDLTKNEIDVVRAG
TAGPGDALYAMLMKWVNKTGRNASIHTLLDALERMEERHAREKIQDLLVD
SGKFIYLEDGTGSAVSLE.

Binding of TRAIL to DR4 leads to trimerization of the receptors and formation of a death inducing signaling complex (DISC) which facilitates the recruitment of procaspase-8/10. In addition to regulating cell death pathways and apoptosis, DR4 also mediates cell survival signals.

As used herein, “cIAP1” (also referred to as “BIRC-2,” “BIRC2,” “cIAP-1,” and “baculoviral IAP repeat containing 2”) refers to a member of the Inhibitor of Apoptosis family of proteins that inhibit apoptosis by interfering with the activation of caspases. The terms cIAP1 and BIRC2 are used interchangeably throughout the specification. CIAP1 is known to directly bind to activated caspase-3 and -7 and inhibit their activities. CIAP1 has been shown to inhibit TNF-induced apoptosis. The human cIAP1 (BIRC2) (isoform 1) amino acid sequence (1-618) (NP_001157) is:

(SEQ ID NO: 3)
MHKTASQRLFPGPSYQNIKSIMEDSTILSDWTNSNKQKMKYDFSCELYRM
STYSTFPAGVPVSERSLARAGFYYTGVNDKVKCFCCGLMLDNWKLGDSPI
QKHKQLYPSCSFIQNLVSASLGSTSKNTSPMRNSFAHSLSPTLEHSSLFS
GSYSSLSPNPLNSRAVEDISSSRTNPYSYAMSTEEARFLTYHMWPLTFLS
PSELARAGFYYIGPGDRVACFACGGKLSNWEPKDDAMSEHRRHFPNCPFL
ENSLETLRFSISNLSMQTHAARMRTFMYWPSSVPVQPEQLASAGFYYVGR
NDDVKCFCCDGGLRCWESGDDPWVEHAKWFPRCEFLIRMKGQEFVDEIQG
RYPHLLEQLLSTSDTTGEENADPPIIHFGPGESSSEDAVMMNTPVVKSAL
EMGFNRDLVKQTVQSKILTTGENYKTVNDIVSALLNAEDEKREEEKEKQA
EEMASDDLSLIRKNRMALFQQLTCVLPILDNLLKANVINKQEHDIIKQKT
QIPLQARELIDTILVKGNAAANIFKNCLKEIDSTLYKNLFVDKNMKYIPT
EDVSGLSLEEQLRRLQEERTCKVCMDKEVSVVFIPCGHLVVCQECAPSLR
KCPICRGIIKGTVRTFLS

### B. Detecting and Measuring Biomarkers

In one embodiment, expression levels of one or more biomarkers measured in a sample are used to derive or calculate a value or score (e.g., which correlates with “high” or “low” expression of the biomarker). This value may be derived solely from these expression levels or optionally derived from a combination of the expression value/score with other components to give a more comprehensive value/score.

Scores for any single one of the biomarkers DR4 (TNFRSF10A) or cIAP1 (BIRC-2) can be used in the methods provided herein. In one embodiment, a patient treated according to the methods described herein has been determined to have a high DR4 score (i.e., high DR4 expression). In another embodiment, a patient treated according to the methods described herein has been determined to have a low cIAP1 score (i.e., low cIAP1 expression). In another embodiment, a patient treated according to the methods described herein has been determined to have a high DR4 score (i.e., high DR4 expression) and a low cIAP1 score (i.e., low cIAP1 expression). In another embodiment, the biomarker score is a ratio of normalized DR4 RNA or protein expression to cIAP RNA or protein expression.

In another embodiment, the patient has a cancer (e.g., colorectal cancer) that has been determined to have a DR4/cIAP1 ratio of at least about 0.5. In one embodiment, the DR4/cIAP1 ratio is at least about 0.5, at least about 0.6, at least about 0.7, at least about 0.8, at least about 0.9, at least about 1.0, at least about 1.1, at least about 1.2, at least about 1.3, at least about 1.4, at least about 1.5, at least about 1.6, at least about 1.7, at least about 1.8, at least about 1.9, or at least about 2.0. In a particular embodiment, the DR4/cIAP1 ratio is at least about 0.5. In another particular embodiment, the DR4/cIAP1 ratio is at least about 0.7.

Various techniques for determining the status of a gene or protein in a tissue or cell sample are known in the art and include, but are not limited to, microarray analysis (e.g., for assaying mRNA or microRNA expression, copy number, etc.), quantitative real-time PCR™ (“qRT-PCR™.”, e.g., TaqMan™), immunoanalysis (e.g., ELISA, immunohistochemistry), etc. The activity level of a polypeptide encoded by a gene may be used in much the same way as the expression level of the gene or polypeptide. Often higher activity levels indicate higher expression levels and while lower activity levels indicate lower expression levels. The methods of the disclosure may be practiced independent of the particular technique used.

In various embodiments, expression of the biomarker is detected at the nucleic acid level. For example, the biomarker score for DR4 (TNFRSF10A) or cIAP1 (BIRC-2) can be assessed based on RNA levels. In the case of measuring RNA levels for the genes, one convenient and sensitive approach is real-time quantitative PCR™ (qPCR) assay, following a reverse transcription reaction. Accordingly, in one embodiment, the method for determining the level of RNA in a sample involves the process of nucleic acid amplification from homogenized tissue, e.g., by RT-PCR (reverse transcribing the RNA and then, amplifying the resulting cDNA employing PCR or any other nucleic acid amplification method), followed by the detection of the amplified molecules. Typically, a cycle threshold (Cₜ) is determined for each test gene and each normalizing gene, i.e., the number of cycles at which the fluorescence from a qPCR reaction above background is detectable.

In one embodiment, RNA expression is assessed by quantitative fluorogenic RT-PCR (qPCR), e.g., by using the TaqMan™ System. Such methods typically utilize pairs of oligonucleotide primers that are specific for the nucleic acid of interest. Further details of such assays are provided below in the Examples.

In another embodiment, the patient sample is determined to have a DR4 RT-PCR score of greater than or equal to −5. In another embodiment, the patient sample is determined to have a DR4 RT-PCR score selected from the group consisting of 5.0, 4,9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0,7, 0.6, 0.5, 0.4, 0,3, 0.2, 0,1, 0, −0.1, −0.2, −0.3, −0.4, −0.5, −0.6, −0.7, −0.8, −0.9, −1.0, −1.1, −1.2, −1,3, −1.4, −1.5, −1.6, −1.7, −1.8, −1.9, −2.0, −2.1, −2.2, −2.3, −2.4, −2.5, −2.6, −2.7, −2.8, −2.9, −3.0, −3.1, −3.2, −3.3, −3.4, −3.5, −3.6, −3.7, −3.8, −3.9, −4.0, −4.1, 4.2, −4.3, −4.4, −4.5, −4.6, −4.7, −4.8, −4.9, −5.0.

In another embodiment, the patient sample is determined to have a cIAP1 RT-PCR score of less than or equal to 5. In another embodiment, the patient sample is determined to have a cIAP1 RT-PCR score selected from the group consisting of 5.0, 4,9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0,7, 0.6, 0.5, 0.4, 0,3, 0.2, 0,1, 0, −0.1, −0.2, −0.3, −0.4, −0.5, −0.6, −0.7, −0.8, −0.9, −1.0, −1.1, −1.2, −1,3, −1.4, −1.5, −1.6, −1.7, −1.8, −1.9, −2.0, −2.1, −2.2, −2.3, −2.4, −2.5, −2.6, −2.7, −2.8, −2.9, −3.0, −3.1, −3.2, −3.3, −3.4, −3.5, −3.6, −3.7, −3.8, −3.9, −4.0, −4.1, 4.2, −4.3, −4.4, −4.5, −4.6, −4.7, −4.8, −4.9, −5.0.

In one embodiment, the expression of one or more normalizing (often called “housekeeping”) genes is also obtained for use in normalizing the expression of test genes. As used herein, “normalizing genes” referred to the genes whose expression is used to calibrate or normalize the measured expression of the gene of interest (e.g., test genes). Importantly, the expression of normalizing genes should be independent of cancer outcome/prognosis, and the expression of the normalizing genes is very similar among all the tumor samples. The normalization ensures accurate comparison of expression of a test gene between different samples. For this purpose, housekeeping genes known in the art can be used. Housekeeping genes are well known in the art, with examples including, but are not limited to, GUSB (glucuronidase, beta), HMBS (hydroxymethylbilane synthase), SDHA (succinate dehydrogenase complex, subunit A, flavoprotein), UBC (ubiquitin C) and YWHAZ (tyrosine 3-monooxygenase/tryptophan 5-monooxygenase activation protein, zeta polypeptide). One or more housekeeping genes can be used. Preferably, at least 2, 5, 10 or 15 housekeeping genes are used to provide a combined normalizing gene set. The amount of gene expression of such normalizing genes can be averaged, combined together by straight additions or by a defined algorithm. In one embodiment, the gene expression is calculated using a geometric mean.

The overall expression of the one or more normalizing genes can be represented by a “normalizing value” which can be generated by combining the expression of all normalizing genes, either weighted equally (straight addition or averaging) or by different predefined coefficients. For example, in a simplest manner, the normalizing value C_{tH} can be the cycle threshold (Cₜ) of one single normalizing gene, or an average of the Cₜ values of 2 or more, preferably 10 or more, or 15 or more normalizing genes, in which case, the predefined coefficient is 1/N, where N is the total number of normalizing genes used. Thus, C_{tH}=(C_{tH1}+C_{tH2}±C_{tHn})/N. As will be apparent to skilled artisans, depending on the normalizing genes used, and the weight desired to be given to each normalizing gene, any coefficients (from 0/N to N/N) can be given to the normalizing genes in weighting the expression of such normalizing genes. That is, C_{tH}=xC_{tH1}+yC_{tH2}±zC_{tHn}, wherein x+y+ . . . +z=1.

In various embodiments, the RNA expression values are calculated as 2^{-ΔΔct}. Δct values are the ct values of the genes of interest normalized to the ct values of housekeeping genes. In one embodiment, ΔΔct values are obtained by normalizing to the Δct values of the reference cell line CCK81.

In another embodiment, the RNA expression is detected using an RNA-ISH assay. For example, RNA may be detected using a chromogenic RNA-In Situ Hybridization Assay (RNA-ISH). A chromogenic RNA-ISH assay may be used to stain an FFPE tissue section for an RNA of interest. For each RNA-ISH assay, a scoring system may be applied by a certified pathologist. The system scores levels as the discrete variables 0, 1+, 2+, 3+, or 4+. FFPE tumor samples may be scored for Biomarker RNA levels using the following variant of an Advanced Cell Diagnostics® (“ACD” Hayward, Calif.) RNAscope® assay. In this assay, cells are permeabilized and incubated with a set of oligonucleotide “Z” probes (see, e.g., U.S. Pat. No. 7,709,198) specific for the biomarker. Using “Z” probes, as well as using multiple sets of probes per transcript, increases the specificity of the assay over standard ISH methods. Following Z probe incubation, a pre-amplifier is added that can only hybridize to a pair of adjacent Z probes bound to the target transcript. This minimizes amplification of non-specific binding. Several sequential amplification steps are then performed based on sequence-specific hybridization to the pre-amplifier, followed by enzyme-mediated chromogenic detection that enables semi-quantitative measurement of biomarker RNA levels in the tumor tissue.

Step 1: FFPE tissue sections are deparaffinized and pretreated to block endogenous phosphatases and peroxidases and to unmask RNA binding sites. Step 2: Target-specific double Z probes are applied, which specifically hybridize to the target RNA at adjacent sequences. Step 3: Targets are detected by sequential applications of a preamplifier oligonucleotide, amplifier oligonucleotides, a final HRP-conjugated oligonucleotide, and DAB. Step 4: Slides are visualized using a light microscope and scored by a pathologist.

To score the assay, a reference tissue microarray (TMA) of four cell lines is stained alongside the tumor sample. These cell lines express different levels of the biomarker, ranging from low to high. A pathologist then assigns the patient sample a score based on a visual comparison with the reference TMA.

In another embodiment, the patient sample is determined to have a DR4 RNA-ISH score of 1+ or higher, 2+ or higher, or 3+ or higher. In another embodiment, the patient sample is determined to have a DR4 RNA-ISH score of 1+ or higher. In another embodiment, the patient sample is determined to have a DR4 RNA-ISH score of 2+ or higher. In another embodiment, the patient sample is determined to have a DR4 RNA-ISH score of 3+ or higher.

In another embodiment, the patient sample is determined to have a cIAP1 RNA-ISH score of 0, 1+ or lower, or 2+ or lower. In another embodiment, the patient sample is determined to have a cIAP1 RNA-ISH score of 0. In another embodiment, the patient sample is determined to have a cIAP1 RNA-ISH score of 1+ or lower. In another embodiment, the patient sample is determined to have a cIAP1 RNA-ISH score of 2+ or lower.

Expression of the biomarker also can be detected at the protein level. Accordingly, the score for DR4 (TNFRSF10A) or cIAP1 (BIRC-2) can be assessed based on detected levels of protein. In a particular embodiment, expression of protein levels is measured using immunohistochemistry (IHC). Immunohistochemistry is a technique for detecting proteins in cells of a tissue section by using antibodies that specifically bind to the proteins. Exemplary IHC assays, such as Fl-IHC and qIHC are well known in the art.

For example, Fl-IHC can be used to measure biomarker levels in tumor cells in FFPE tissue. Fl-IHC is an imaging-based assay and provides a measure of the number of protein molecules per tumor cell in each sample. Either surgically resected tumor tissue or core needle biopsies are collected from patients, fixed in formalin, and embedded in paraffin blocks using standard procedures. FFPE blocks are sectioned, mounted on glass slides, and co-stained for DNA, cytokeratin (CK), and the biomarker(s). Final detection of the markers is based on fluorescence. The slides may be imaged using an Aperio® ScanScope® FL set at 20×magnification and analyzed using quantitative digital image analysis. The automated image analysis algorithm applies a regular grid across the tissue region where each square tile is approximately the size of a single cell. The fluorescence measurements of the CK+tiles are then converted to an absolute scale (receptors per cell) using a standard curve generated from a tissue microarray (TMA), stained and imaged at the same time as the patient sample, that is composed of cell lines with known biomarker protein levels.

qIHC can also be used to detect protein levels of the biomarkers. qIHC uses a standard brown-stain technology to indicate protein levels in FFPE tissue sections. A TMA-based scoring system may be applied by a certified pathologist. This system yields scores based on staining intensity (e.g., 0, 1, 2, 3, 4).

In one embodiment, the patient sample is determined to have a DR4 IHC score of 1 or higher, 2 or higher, or 3 or higher. In another embodiment, the patient sample is determined to have a DR4 IHC score of 1 or higher. In another embodiment, the patient sample is determined to have a DR4 IHC score of 2 or higher. In another embodiment, the patient sample is determined to have a DR4 IHC score of 3 or higher.

In another embodiment, the patient sample is determined to have a cIAP1 IHC score of 0, 1 or lower, or 2 or lower. In another embodiment, the patient sample is determined to have a cIAP1 IHC score of 0. In another embodiment, the patient sample is determined to have a cIAP1

IHC score of 1 or lower. In another embodiment, the patient sample is determined to have a cIAP1 IHC score of 2 or lower.

In another aspect, methods of identifying cancer patients (e.g., colorectal cancer patients) who will benefit from treatment with a TRAIL-based therapeutic of a death receptor agonist based on their particular levels of DR4 and/or cIAP1 expression are provided, wherein the identification is based only on levels of DR4 and/cIAP1. For example, the method does not involve measuring levels of one or more of the following biomarkers: APAF1, CASP3, CASP6, CASP7, CASP8, CASP9, CASP10, FADD, SPTAN1, BCL2, GAS2, BID, LMNA, CFLAR, MAP3K14, BIRC3, CHUK, NFKB1, NFKBIA, TNFRSF10B, TNFSF25, TNFSF10, TNFSF12, CYCS, DFFA, DFFB, RELA, TRADD, RIPK1, TRAF2, and XIAP. In another embodiment, the method includes measuring levels of DR4 and/cIAP1 and one or more additional biomarkers, wherein the one or more additional biomarkers is not APAF1, CASP3, CASP6, CASP7, CASP8, CASP9, CASP10, FADD, SPTAN1, BCL2, GAS2, BID, LMNA, CFLAR, MAP3K14, BIRC3, CHUK, NFKB1, NFKBIA, TNFRSF10B, TNFSF25, TNFSF10, TNFSF12, CYCS, DFFA, DFFB, RELA, TRADD, RIPK1, TRAF2, and/or XIAP.

In another aspect, methods of treating a cancer patient who has been determined to have particular levels of one or more biomarkers (DR4 and/or cIAP1) by administering a TRAIL-based therapeutic or death receptor agonist are provided, wherein the decision to treat a patient is based only on levels of Dr4 and/or cIAP1. For example, the patient's levels of one or more of the following biomarkers have not been assessed as part of the method: APAF1, CASP3, CASP6, CASP7, CASP8, CASP9, CASP10, FADD, SPTAN1, BCL2, GAS2, BID, LMNA, CFLAR, MAP3K14, BIRC3, CHUK, NFKB1, NFKBIA, TNFRSF10B, TNFSF25, TNFSF10, TNFSF12, CYCS, DFFA, DFFB, RELA, TRADD, RIPK1, TRAF2, and XIAP. In another embodiment, the decision to treat a patient is based on levels of Dr4 and/or cIAP1 and one or more additional biomarkers, wherein the one or more additional biomarkers is not APAF1, CASP3, CASP6, CASP7, CASP8, CASP9, CASP10, FADD, SPTAN1, BCL2, GAS2, BID, LMNA, CFLAR, MAP3K14, BIRC3, CHUK, NFKB1, NFKBIA, TNFRSF10B, TNFSF25, TNFSF10, TNFSF12, CYCS, DFFA, DFFB, RELA, TRADD, RIPK1, TRAF2, and/or XIAP.

For example, in one embodiment, the one or more biomarkers is not APAF1. In another embodiment, the one or more biomarkers is not CASP3. In another embodiment, the one or more biomarkers is not CASP6. In another embodiment, the one or more biomarkers is not CASP7. In another embodiment, the one or more biomarkers is not CASP8. In another embodiment, the one or more biomarkers is not CASP9. In another embodiment, the one or more biomarkers is not CASP10. In another embodiment, the one or more biomarkers is not FADD. In another embodiment, the one or more biomarkers is not SPTAN1. In another embodiment, the one or more biomarkers is not comprise BCL2. In another embodiment, the one or more biomarkers is not GAS2. In another embodiment, the one or more biomarkers is not BID. In another embodiment, the one or more biomarkers is not LMNA. In another embodiment, the one or more biomarkers is not CFLAR. In another embodiment, the one or more biomarkers is not MAP3K14. In another embodiment, the one or more biomarkers is not BIRC3. In another embodiment, the one or more biomarkers is not CHUK. In another embodiment, the one or more biomarkers is not NFKB1. In another embodiment, the one or more biomarkers is not NFKBIA. In another embodiment, the one or more biomarkers is not TNFRSF10B. In another embodiment, the one or more biomarkers do not comprise TNFSF25. In another embodiment, the one or more biomarkers is not TNFSF10. In another embodiment, the one or more biomarkers is not TNFSF12. In another embodiment, the one or more biomarkers is not CYCS. In another embodiment, the one or more biomarkers is not DFFA. In another embodiment, the one or more biomarkers is not DFFB. In another embodiment, the one or more biomarkers is not RELA. In another embodiment, the one or more biomarkers is not TRADD. In another embodiment, the one or more biomarkers is not RIPK1. In another embodiment, the one or more biomarkers is not TRAF2. In another embodiment, the one or more biomarkers is not comprise XIAP.

### C. Biological Samples

The expression of one or more biomarkers (e.g., DR4 or cIAP1) may be determined in a biological sample (biopsy) obtained from a subject.

As used herein, “tumor sample” refers to any biological sample containing one or more tumor cells, or one or more tumor derived RNA or protein, and obtained from a patient (e.g., a cancer patient). For example, a tissue sample obtained from a tumor tissue of a cancer patient is a useful tumor sample in the methods described herein. Preferably, the sample contains largely tumor cells. A single malignant cell from a cancer patient's tumor is also a useful tumor sample. Such a malignant cell can be obtained directly from the patient's tumor, or purified from the patient's bodily fluid (e.g., blood, urine). Thus, a bodily fluid such as blood, urine, sputum and saliva containing one or tumor cells, or tumor-derived RNA or proteins, can also be useful as a tumor sample for purposes of the methods described herein. Such a sample is typically further processed after it is obtained from the subject. Biopsy samples suitable for detecting and quantitating the biomarkers described herein may be fresh, frozen, or fixed. Suitable samples are preferably sectioned. Alternatively, samples may be solubilized and/or homogenized and subsequently analyzed. In one embodiment, a freshly obtained biopsy sample embedded in a cryoprotectant such as OCT® or Cryomatrix® and frozen using, for example, liquid nitrogen or difluorodichloromethane. The frozen sample is serially sectioned in a cryostat. In another embodiment, samples are fixed and embedded prior to sectioning. For example, a tissue sample may be fixed in, for example, formalin, gluteraldehyde, ethanol or methanol, serially dehydrated (e.g., using alcohol and or xylenes) and embedded in, for example, paraffin.

In another embodiment, the sample is a microtome section of a biopsy (e.g., FFPE prior to microtome sectioning). In another embodiment, the biopsy was obtained within 30, 60, or 90 days prior to treating the patient.

In another embodiment, the tumor sample is derived from circulating tumor cells in the blood. Circulating tumor cells are cells that have detached from a primary tumor and entered the vascular system. These may be found in frequencies on the order of 1-10 CTC per mL of whole blood in patients with metastatic disease and the isolation of these cells may offer a non-invasive alternative to tumor biopsies and may often be used in cases where a procuring a biopsy sample isn't possible.

### D. TRAIL-Based Therapeutics

### i. TRAIL Polypeptides

In additional to rhTRAIL, TRAIL polypeptides as described in international application number PCT/US2017/022789 (which is expressly incorporated herein by reference) are also useful in the methods described herein. TRAIL polypeptides may be TRAIL monomers, dimers, or trimers in a single polypeptide chain construct, regardless of precise format or fusion partner (if any). For example, a single chain TRAIL construct can comprise one, two, or three TRAIL monomers.

In one embodiment, the TRAIL polypeptide comprises one TRAIL domain (monomer). In another embodiment, the TRAIL polypeptide comprises two TRAIL monomers (dimer). In another embodiment, the polypeptide comprises three TRAIL monomers (trimer). In another embodiment, the polypeptide comprises the amino acid residues 95-281, 114-281, or 120-281 of SEQ ID NO: 1. In another embodiment, the polypeptide comprises a TRAIL polypeptide linked (e.g., fused) to an antibody Fc region or a fragment thereof and/or a Fab or fragment thereof and/or an antibody and/or an albumin (e.g., HSA).

In one embodiment, the TRAIL monomer comprises full-length human TRAIL (i.e., amino acid residues 1-281 of SEQ ID NO: 1). In another embodiment, the TRAIL monomer comprises a portion of the amino acid sequence set forth in SEQ ID NO: 1. In another embodiment, the TRAIL monomer comprises amino acids 114-281 of SEQ ID NO: 1. In another embodiment, the TRAIL monomer consists of amino acids 114-281 of SEQ ID NO: 1. In another embodiment, the TRAIL domain comprises amino acid residues 95-281 of SEQ ID NO: 1. In another embodiment, the TRAIL monomer consists of amino acid residues 95-281 of SEQ ID NO: 1. In another embodiment, the TRAIL monomer comprises amino acid residues 120-281 of SEQ ID NO: 1. In another embodiment, the TRAIL monomer consists of amino acid residues 120-281 of SEQ ID NO: 1.

In another embodiment, the TRAIL domain consists of or comprises amino acid residues 90-281 of SEQ ID NO: 1. In another embodiment, the TRAIL domain consists of or comprises amino acid residues 91-281 of SEQ ID NO: 1. In another embodiment, the TRAIL domain consists of or comprises amino acid residues 92-281 of SEQ ID NO: 1. In another embodiment, the TRAIL domain consists of or comprises amino acid residues 93-281 of SEQ ID NO: 1. In another embodiment, the TRAIL domain consists of or comprises amino acid residues 94-281 of SEQ ID NO: 1. In another embodiment, the TRAIL domain consists of or comprises amino acid residues 95-281 of SEQ ID NO: 1. In another embodiment, the TRAIL domain consists of or comprises amino acid residues 96-281 of SEQ ID NO: 1. In another embodiment, the TRAIL domain consists of or comprises amino acid residues 97-281 of SEQ ID NO: 1. In another embodiment, the TRAIL domain consists of or comprises amino acid residues 98-281 of SEQ ID NO: 1. In another embodiment, the TRAIL domain consists of or comprises amino acid residues 99-281 of SEQ ID NO: 1. In another embodiment, the TRAIL domain consists of or comprises amino acid residues 100-281 of SEQ ID NO: 1. In another embodiment, the TRAIL domain consists of or comprises amino acid residues 101-281 of SEQ ID NO: 1. In another embodiment, the TRAIL domain consists of or comprises amino acid residues 102-281 of SEQ ID NO: 1. In another embodiment, the TRAIL domain consists of or comprises amino acid residues 103-281 of SEQ ID NO: 1. In another embodiment, the TRAIL domain consists of or comprises amino acid residues 104-281 of SEQ ID NO: 1. In another embodiment, the TRAIL domain consists of or comprises amino acid residues 105-281 of SEQ ID NO: 1. In another embodiment, the TRAIL domain consists of or comprises amino acid residues 106-281 of SEQ ID NO: 1. In another embodiment, the TRAIL domain consists of or comprises amino acid residues 107-281 of SEQ ID NO: 1. In another embodiment, the TRAIL domain consists of or comprises amino acid residues 108-281 of SEQ ID NO: 1. In another embodiment, the TRAIL domain consists of or comprises amino acid residues 109-281 of SEQ ID NO: 1. In another embodiment, the TRAIL domain consists of or comprises amino acid residues 110-281 of SEQ ID NO: 1. In another embodiment, the TRAIL domain consists of or comprises amino acid residues 111-281 of SEQ ID NO: 1. In another embodiment, the TRAIL domain consists of or comprises amino acid residues 112-281 of SEQ ID NO: 1. In another embodiment, the TRAIL domain consists of or comprises amino acid residues 113-281 of SEQ ID NO: 1. In another embodiment, the TRAIL domain consists of or comprises amino acid residues 114-281 of SEQ ID NO: 1. In another embodiment, the TRAIL domain consists of or comprises amino acid residues 115-281 of SEQ ID NO: 1. In another embodiment, the TRAIL domain consists of or comprises amino acid residues 116-281 of SEQ ID NO: 1. In another embodiment, the TRAIL domain consists of or comprises amino acid residues 117-281 of SEQ ID NO: 1. In another embodiment, the TRAIL domain consists of or comprises amino acid residues 118-281 of SEQ ID NO: 1. In another embodiment, the TRAIL domain consists of or comprises amino acid residues 119-281 of SEQ ID NO: 1. In another embodiment, the TRAIL domain consists of or comprises amino acid residues 120-281 of SEQ ID NO: 1. In another embodiment, the TRAIL domain consists of or comprises amino acid residues 121-281 of SEQ ID NO: 1. In another embodiment, the TRAIL domain consists of or comprises amino acid residues 122-281 of SEQ ID NO: 1. In another embodiment, the TRAIL domain consists of or comprises amino acid residues 123-281 of SEQ ID NO: 1. In another embodiment, the TRAIL domain consists of or comprises amino acid residues 124-281 of SEQ ID NO: 1. In another embodiment, the TRAIL domain consists of or comprises amino acid residues 125-281 of SEQ ID NO: 1. In another embodiment, the TRAIL domain consists of or comprises amino acid residues 126-281 of SEQ ID NO: 1. In another embodiment, the TRAIL domain consists of or comprises amino acid residues 127-281 of SEQ ID NO: 1. In another embodiment, the TRAIL domain consists of or comprises amino acid residues 128-281 of SEQ ID NO: 1. In another embodiment, the TRAIL domain consists of or comprises amino acid residues 129-281 of SEQ ID NO: 1. In another embodiment, the TRAIL domain consists of or comprises amino acid residues 130-281 of SEQ ID NO: 1.

In another embodiment, the TRAIL monomer comprises or consists of a sequence at least 80%, 85%, 90%, 91%, 92%, 93%, 94%, 95%, 96%, 97%, 98%, or 99% identical to a sequence having an N-terminus at any one of amino acid residues 90-130 of SEQ ID NO: 1 and a C terminus at any one of amino acid residues 251-281 of SEQ ID NO: 1.

In another embodiment, the TRAIL monomer comprises no more than about 250 amino acid residues, preferably no more than about 200 amino acid residues, and more preferably no more than about 150 amino acid residues. In another embodiment, the TRAIL monomer consists of no more than about 250 amino acid residues, preferably no more than about 200 amino acid residues, and more preferably no more than about 150 amino acid residues.

In one embodiment, the fusion polypeptide comprises a set of three human TRAIL monomers to form a single-chain TRAIL trimer. In one embodiment, the single-chain TRAIL trimer comprises, in amino- to carboxyl-terminal order, a first TRAIL monomer, a linker, a second TRAIL monomer, a second linker, and a third TRAIL monomer. In one embodiment, the linker comprises a G₄S domain (SEQ ID NO: 40). In another embodiment, each linker consists of 15-20 amino acids. In another embodiment, each of the two inter-TRAIL monomer linkers comprises 3 G₄S domains (SEQ ID NO: 50).

In one embodiment, the TRAIL fusion polypeptide is an Fc-TRAIL fusion polypeptide. In another embodiment the TRAIL fusion polypeptide is a Fab-TRAIL fusion polypeptide. In yet another embodiment the TRAIL fusion polypeptide is an HSA-TRAIL fusion polypeptide. Suitable human serum albumin (HSA) moieties for use in such an HSA-TRAIL fusion polypeptide include native and mutant HSAs disclosed in U.S. Pat. Nos. 8,927,694 and 8,877,687.

In one embodiment, the TRAIL polypeptide binds to at least one of its signaling receptors (specifically DR4 and DR5) or non-signaling decoy receptors, DcR1, DcR2, and osteoprotegrin (OPG). In another embodiment, the TRAIL polypeptide induces apoptosis.

Each TRAIL monomer may contain a mutation or combination of mutations can be independently present or absent from each of the three monomers. In one embodiment, the TRAIL mutations may be selected from amino acid substitution at one or more of positions 121, 130, 213, 215, 228, and 247 of SEQ ID NO: 1. As provided in PCT/US2017/022789 (which is expressly incorporated herein by reference), beneficial mutations in TRAIL monomer for use in a single chain TRAIL molecule include individual mutations (numbered per SEQ ID NO: 1, above) as follows: R121I, R130G, Y213W, S215D, N228S and I247V.

In one aspect, each of the three monomers contains the same mutation or the same combination of mutations, in another aspect two of the three monomers contains the same mutation or the same combination of mutations, while the third comprises a different mutation or combination of mutations, or no mutation, and in yet another aspect, each of the three monomers comprises a different mutation or combination of mutations, or no mutation is present in one or two of the three monomers. For example, exemplary single chain mutant TRAIL trimers may be selected from “T148”, “T151”, “T153”, “T182”, “T183”, “T186”, “T191”, “T196”, “T202”, “T203”, “T204”, “T205”, “T206”, “T207”, “T208”, “T209”, “T210”, and “T211” (SEQ ID NO: 4-21, respectively).

Provided herein are methods of treating a patient having cancer with high levels of DR4 and low levels of cIAP1, the method comprising administering a therapeutically effective amount of a TRAIL-based therapeutic or a death receptor agonist. In one embodiment, the TRAIL-based therapeutic is rhTRAIL. In another embodiment, the TRAIL-based therapeutic is a TRAIL monomer, dimer, or trimer. In another embodiment, the TRAIL-based therapeutic is T148 (SEQ ID NO: 4). In another embodiment, the TRAIL-based therapeutic is T151 (SEQ ID NO: 5). In another embodiment, the TRAIL-based therapeutic is T153 (SEQ ID NO: 6). In another embodiment, the TRAIL-based therapeutic is T182 (SEQ ID NO: 7). In another embodiment, the TRAIL-based therapeutic is T183 (SEQ ID NO: 8). In another embodiment, the TRAIL-based therapeutic is T186 (SEQ ID NO: 9). In another embodiment, the TRAIL-based therapeutic is T191 (SEQ ID NO: 10). In another embodiment, the TRAIL-based therapeutic is T196 (SEQ ID NO: 11). In another embodiment, the TRAIL-based therapeutic is T202 (SEQ ID NO: 12). In another embodiment, the TRAIL-based therapeutic is T203 (SEQ ID NO: 13). In another embodiment, the TRAIL-based therapeutic is T204 (SEQ ID NO: 14). In another embodiment, the TRAIL-based therapeutic is T205 (SEQ ID NO 15). In another embodiment, the TRAIL-based therapeutic is T206 (SEQ ID NO: 16). In another embodiment, the TRAIL-based therapeutic is T207 (SEQ ID NO: 17). In another embodiment, the TRAIL-based therapeutic is T208 (SEQ ID NO: 18). In another embodiment, the TRAIL-based therapeutic is T209 (SEQ ID NO 19). In another embodiment, the TRAIL-based therapeutic is T210 (SEQ ID NO: 20). In another embodiment, the TRAIL-based therapeutic is T211 (SEQ ID NO: 21).

Other known TRAIL-based polypeptides and ligands can be used in the methods described herein (e.g., such as those described in U.S. Pat. No. 9,359,420, which is expressly incorporated herein by reference).

### ii. Fc-TRAIL Fusion Polypeptides

TRAIL-based therapies useful in the methods described herein may also comprise TRAIL polypeptides linked to an Fc region or fragment thereof.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (C_{H2}) and third (C_{H3}) constant domains of the antibody's two heavy chains; IgM and IgE Fc regions comprise three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or amino acid between these two amino acids) to the carboxy-terminus of the heavy chain, wherein the numbering is according to the EU index as in Kabat. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG. As used herein, the Fc region may be a native sequence Fc, including any allotypic variant, or a variant Fc (e.g., a non-naturally occurring Fc). Fc may also refer to this region in isolation or in the context of an Fc-comprising protein polypeptide such as a “binding protein comprising an Fc region,” also referred to as an “Fc fusion protein” (e.g., an antibody or immunoadhesin).

In another embodiment, the Fc-TRAIL fusion polypeptide comprises a native sequence Fc region. A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1:1).

In certain embodiments, the Fc region is a variant Fc region, e.g., an Fc sequence that has been modified (e.g, by amino acid substitution, deletion and/or insertion) relative to a parent Fc sequence (e.g., an unmodified Fc polypeptide that is subsequently modified to generate a variant), to provide desirable structural features and/or biological activity.

For example, one may make modifications in the Fc region in order to generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fe. Such Fe region variants will generally comprise at least one amino acid modification in the Fc region. Combining amino acid modifications is thought to be particularly desirable. For example, the variant Fc region may include two, three, four, five, etc. substitutions therein, e.g. of the specific Fe region positions identified herein.

A variant Fe region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fe domain that is held together non-covalently. In other embodiments, the Fe region may be modified to make it more compatible with a selected host cell. For example, one may remove the PA sequence near the N-terminus of a typical native Fe region, which may be recognized by a digestive enzyme in E. coli such as proline iminopeptidase. in other embodiments, one or more glycosylation sites within the Fc domain may be removed. Residues that are typically glycosylated (e.g., asparagine) may confer cytolytic response. Such residues may be deleted or substituted with unglyeosylated residues (e.g., alanine). In other embodiments, sites involved in interaction with complement, such as the C1q binding site, may be removed from the Fe region. For example, one may delete or substitute the EKK sequence of human IgG1. In certain embodiments, sites that affect binding to Fe receptors may be removed, preferably sites other than salvage receptor binding sites. In other embodiments, an Fe region may be modified to remove an ADCC site. ADCC sites are known in the art; see, for example, Molec. Immunol. 29 (5): 633-9 (1992) with regard to ADCC sites in IgG 1. Specific examples of variant Fe domains are disclosed for example, in WO 97/34631 and WO 96/32478.

In one embodiment, the hinge region of Fc is modified such that the number of cysteine residues in the hinge region is altered, e.g., increased or decreased. This approach is described further in U.S. Patent No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. Nos. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase antibody dependent cellular cytotoxicity (ADCC) and/or to increase the affinity for an Fcγreceptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268E, 324T, 332D, and 332E. Exemplary variants include 239D/332E, 236A/332E, 236A/239D/332E, 268F/324T, 267E/ 268F, 267E/324T, and 267E/268F/324T. Other modifications for enhancing FeyR and complement interactions include but are not limited to substitutions 298A, 333A, 334A, 326A, 2471, 339D, 339Q, 28011, 290S, 298D, 2985, 2431., 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249 252, 254 255, 256. 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fe region, wherein the numbering of the residues in the Fc region is that of the EU index as in Kabat (WO00/42072).

Other Fc modifications that can be made to Fes are those for reducing or ablating binding to FcγR and/or complement proteins, thereby reducing or ablating Fc-mediated effector functions such as ADCC. ADCP, and CDC. Exemplary modifications include but are not limited substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 2361Z/328R. Other modifications for reducing FcyR and complement interactions include substitutions 297A, 234A, 235A, 237A, 318A, 228P, 236E, 268Q, 309L, 330S, 331 S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fe region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCT Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

Fc variants that enhance affinity for an inhibitory receptor Fcy Rllb may also be used. Such variants may provide an Fc fusion protein with immunomodulatory activities related to FcyRllb⁺cells, including for example B cells and monocytes. In one embodiment, the Fc variants provide selectively enhanced affinity to FcyRllb relative to one or more activating receptors. Modifications for altering binding to FcyRllb include one or more modifications at a position selected from the group consisting of 234, 235, 236, 237, 239, 266, 267, 268, 325, 326, 327, 328, and 332, according to the EU index. Exemplary substitutions for enhancing FcyRllb affinity include but are not limited to 234D, 234E, 234F, 234W, 235D, 235F, 235R, 235Y, 236D, 236N, 237D, 237N, 239D, 239E, 266M, 267D, 267E, 268F), 268E, 327E), 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcyRllb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art including but not limited to, equilibrium methods (e.g., enzyme-linked immunoabsorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In certain embodiments, the antibody is modified to increase its biological half-life. Various approaches are possible. For example, this may be done by increasing the binding affinity of the Fc region for FcRn. For example, one or more of more of following residues can be mutated: 252, 254, 256, 433, 435, and 436, as described in U.S. Pat. No, 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FeRn and/or improve pharmacokinetic properties include substitutions at positions 259, 308, 428, and 434, including for example 259I, 308F, 428L, 428M, 434S, 434H, 434F, 434Y, and 434M. Other variants that increase Fe binding to FcRn include: 250E, 250Q, 428L, 428F, 250Q/428L (Hinton et al., 2004, J. Biol. Chem. 279(8): 6213-6216, Hinton et al, 2006 Journal of Immunology 176:346-356), 256A, 272A, 286A, 305A, 307A, 307Q, 31 1A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 31 1 5, 433R, 433S, 433I, 433P, 433Q, 434H, 434F, 434Y, 252Y/254T/256E, 433K/434F/436H, 308T/309P/311S (Dall Acqua et al. Journal of Immunology, 2002, 169:5171-5180, Dall Acqua et al., 2006, Journal of Biological Chemistry 281:23514-23524). Other modifications for modulating FeRn binding are described in Yeung et al., 2010, J Immunol, 182:7663-7671. In certain embodiments, hybrid IgG isotypes with particular biological characteristics may be used. For example, an IgG1AgG3 hybrid variant may be constructed by substituting IgG1 positions in the CH2 and/or CH3 region with the amino acids from IgG3 at positions where the two isotypes differ. Thus a hybrid variant IgG antibody, may be constructed that comprises one or more substitutions, e.g., 274Q, 276K, 300F, 339T, 356E, 358M, 384S, 392N, 397M, 4221, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed that comprises one or more substitutions, e.g., one or more of the following amino acid substitutions: 233E, 234L, 235L, −236G (referring to an insertion of a glycine at position 236), and 327A.

Moreover, the binding sites on human IgG1 for FcγR1, FcγRII, FcγRIII and FcRn have been mapped and variants with improved binding have been described (see Shields, R.L. et al. (2001) J. Biol. Chem. 276:6591-6604). Specific mutations at positions 256, 290, 298, 333, 334 and 339 were shown to improve binding to FcγRIII Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/I332E, S239D/I332E/A330L, L235V/F243L/R292P/Y300L/P396L, and M428L/N434S.

In another embodiment, an Fc-TRAIL polypeptide chain is dimerized to a second Fc-TRAIL polypeptide chain. In a particular embodiment, the two Fc-TRAIL polypeptide chains are dimerized by at least one inter-Fc disulfide bond. In another embodiment, the two Fc-TRAIL polypeptide chains are dimerized by at least two inter-Fc disulfide bonds. In another embodiment, the two Fc-TRAIL polypeptide chains are dimerized by at least three inter-Fc disulfide bonds.

In a particular embodiment, the Fc-TRAIL fusion polypeptide comprises two polypeptide chains dimerized by at least one inter-Fc disulfide bond, each chain comprising a human IgG Fc moiety peptide-bound to a set of three human TRAIL domains to form a single unbranched polypeptide comprising, in amino- to carboxyl-terminal order, the Fc moiety, a linker, a first TRAIL monomer, an inter-monomer linker, a second TRAIL monomer, a second inter-monomer linker, and a third TRAIL monomer, wherein each linker consists of 15-20 amino acids and each of the two inter-TRAIL monomer linkers comprises 3 G₄S motifs.

In another embodiment, the Fc region is modified with respect to effector function, so as to enhance the effectiveness of the polypeptide in treating a disease, e.g., cancer. For example cysteine residue(s) may be introduced in the Fc region, thereby allowing inter-chain disulfide bond formation in this region. The homodimeric polypeptide thus generated may have improved internalization capability and/or increased complement-mediated cell killing and antibody-dependent cellular cytotoxicity (ADCC). Homodimeric polypeptides with enhanced anti-tumor activity may also be prepared using heterobifunctional cross-linkers. Alternatively, a polypeptide can be engineered which has dual Fc regions and may thereby have enhanced complement lysis and ADCC capabilities.

In a particular embodiment, the Fc-TRAIL fusion polypeptide comprises a human IgG Fc moiety, or fragment thereof, bound to a set of three human TRAIL domains to form a single unbranched polypeptide comprising, in amino- to carboxyl-terminal order, the Fc moiety, a linker, a first TRAIL monomer, an inter-monomer linker, a second TRAIL monomer, a second inter-monomer linker, and a third TRAIL monomer. In a particular embodiment, for example, the Fc-TRAIL fusion polypeptide comprises any one of SEQ ID NO: 22-39. In another embodiment, the Fc-TRAIL fusion polypeptide comprises at least one, two, three, or four mutations not found in native wild-type human TRAIL.

Exemplary single chain mutant Fc-TRAIL fusion polypeptides may be selected from “Fc-T148”, “Fc-T151”, “Fc-T153”, “Fc-T182”, “Fc-T183”, “Fc-T186”, “Fc-T191”, “Fc-T196”, “Fc-T202”, “Fc-T203”, “Fc-T204”, “Fc-T205”, “Fc-T206”, “Fc-T207”, “Fc-T208”, “Fc-T209”, “Fc-T210”, and “Fc-T211” (SEQ ID NO: 22-39, respectively). In another embodiment, the Fc-TRAIL fusion polypeptide is T148 (SEQ ID NO: 22). In another embodiment, the Fc-TRAIL fusion polypeptide is Fc-T151 (SEQ ID NO: 23). In another embodiment, the Fc-TRAIL fusion polypeptide is Fc-T153 (SEQ ID NO: 24). In another embodiment, the Fc-TRAIL fusion polypeptide is Fc-T182 (SEQ ID NO: 25). In another embodiment, the Fc-TRAIL fusion polypeptide is Fc-T183 (SEQ ID NO: 26). In another embodiment, the Fc-TRAIL fusion polypeptide is Fc-T186 (SEQ ID NO: 27). In another embodiment, the Fc-TRAIL fusion polypeptide is Fc-T191 (SEQ ID NO: 28). In another embodiment, the Fc-TRAIL fusion polypeptide is Fc-T196 (SEQ ID NO: 29). In another embodiment, the Fc-TRAIL fusion polypeptide is Fc-T202 (SEQ ID NO: 30). In another embodiment, the Fc-TRAIL fusion polypeptide is Fc-T203 (SEQ ID NO: 31). In another embodiment, the Fc-TRAIL fusion polypeptide is Fc-T204 (SEQ ID NO: 32). In another embodiment, the Fc-TRAIL fusion polypeptide is Fc-T205 (SEQ ID NO: 33). In another embodiment, the Fc-TRAIL fusion polypeptide is Fc-T206 (SEQ ID NO: 34). In another embodiment, the Fc-TRAIL fusion polypeptide is Fc-T207 (SEQ ID NO: 35). In another embodiment, the Fc-TRAIL fusion polypeptide is Fc-T208 (SEQ ID NO: 36). In another embodiment, the Fc-TRAIL fusion polypeptide is Fc-T209 (SEQ ID NO: 37). In another embodiment, the Fc-TRAIL fusion polypeptide is Fc-T210 (SEQ ID NO: 38). In another embodiment, the Fc-TRAIL fusion polypeptide is Fc-T211 (SEQ ID NO: 39).

In one embodiment, the Fc-TRAIL fusion polypeptide induces cancer cell apoptosis.

Other known TRAIL-based therapies and Fc-fusions can be used in the methods described herein. For example, in one embodiment, the TRAIL-based therapeutic is a hexavalent Fc-fusion protein (e.g., ABBV-621) (see also ClinicalTrials.gov identifier NCT03082209, the contents of which are expressly incorporated herein by reference). ABBV-621 is a TRAIL-receptor agonist consisting of six receptor binding domains of TRAIL, fused to the Fc-domain of a human IgG1 antibody.

In addition, Fc-fusion polypeptides that can be used in the methods described herein included single-chain trail-receptor agonists (e.g., such as those described in WO2015/164588 and US2015/0337027, the contents of which are expressly incorporated herein by reference).

### iii. Fab-Fc-TRAIL and Fab-TRAIL Fusion Polypeptides

The Fc-TRAIL fusion polypeptides described herein may further comprise an antibody Fab region, or fragment thereof (e.g., Fab-Fc-TRAIL fusion polypeptide). “Fab” refers to the antigen binding portion of an antibody, comprising two chains: a first chain that comprises a VH domain and a CH1 domain and a second chain that comprises a VL domain and a CL domain. Although a Fab is typically described as the N-terminal fragment of an antibody that was treated with papain and comprises a portion of the hinge region, it is also used herein as referring to a binding domain wherein the heavy chain does not comprise a portion of the hinge. In another embodiment, the TRAIL fusion comprises a full-length heavy and light chain, or fragment thereof. In another embodiment the TRAIL fusion comprises a full-length antibody.

In one embodiment, the Fab-Fc-TRAIL fusion or the full-length heavy and light chain heavy chain TRAIL fusion, or fragment thereof, can be dimerized to a second fusion polypeptide chain. In a particular embodiment, the two fusion polypeptide chains are dimerized by at least one inter-Fc disulfide bond. In another embodiment, the two fusion polypeptide chains are dimerized by at least two inter-Fc disulfide bonds. In another embodiment, the two fusion polypeptide chains are dimerized by at least three inter-Fc disulfide bonds.

In another embodiment the Fab-Fc, heavy and light chain, full-length antibody, or fragment thereof, is fused to a TRAIL polypeptide with a linker. In another embodiment the linker is an amino acid linker. Modifications can also be made within one or more of the framework or joining regions of the heavy and/or the light chain variable regions of the Fab region or antibody, so long as antigen binding affinity subsequent to these modifications is maintained.

In another embodiment, the Fab-Fc-TRAIL fusion polypeptide comprises a human Fab moiety, or fragment thereof, bound to a human Fc moiety, or fragment thereof, bound to a set of three human TRAIL monomers to form a single unbranched polypeptide comprising, in amino-to carboxyl-terminal order, the Fc moiety, a linker, a first TRAIL monomer, an inter-monomer linker, a second TRAIL monomer, a second inter-monomer linker, and a third TRAIL monomer. In another embodiment, the Fab-Fc-TRAIL fusion polypeptide comprises at least one, two, three, or four mutations not found in native wild-type human TRAIL.

The TRAIL fusions describe herein, may also comprise an antibody Fab region, or antigen-binding portion thereof (Fab-TRAIL). In one embodiment the Fab region comprises a full-length heavy chain. In another embodiment, the Fab region comprises a full-length heavy and light chain, or fragment thereof. In another embodiment, the Fab-TRAIL fusion, can be dimerized to a second fusion polypeptide chain. In a particular embodiment, the two fusion polypeptide chains are dimerized by at least one inter-Fc disulfide bond. In another embodiment, the two fusion polypeptide chains are dimerized by at least two inter-Fc disulfide bonds. In another embodiment, the two fusion polypeptide chains are dimerized by at least three inter-Fc disulfide bonds.

In another embodiment the Fab, or fragment thereof, is fused to a TRAIL polypeptide with a linker. In another embodiment the linker is an amino acid linker. Modifications can also be made within one or more of the framework or joining regions of the heavy and/or the light chain variable regions of the Fab region or antibody, so long as antigen binding affinity subsequent to these modifications is maintained.

In another embodiment, the Fab-TRAIL fusion polypeptide comprises a human Fab moiety, or fragment thereof, bound to a set of three human TRAIL monomers to form a single unbranched polypeptide comprising, in amino- to carboxyl-terminal order, the Fab moiety, a linker, a first TRAIL monomer, an inter-monomer linker, a second TRAIL monomer, a second inter-monomer linker, and a third TRAIL monomer. In another embodiment, the Fab-TRAIL fusion polypeptide comprises at least one, two, three, or four mutations not found in native wild-type human TRAIL. An exemplary Fab-TRAIL fusion polypeptide may comprise an anti-EpCAM Fab fused to a soluble TRAIL (scTRAIL) polypeptide.

### iv. Albumin-TRAIL Fusion Polypeptides

In another embodiment, a TRAIL polypeptide is linked to an albumin moiety (e.g., Human Serum Albumin (HSA)). In another embodiment, the albumin-TRAIL fusion polypeptide comprises one, two, or three TRAIL monomers.

In a particular embodiment, a single TRAIL fusion polypeptide chain comprises a human serum albumin moiety peptide-bound to a set of three human TRAIL monomers to form a single unbranched polypeptide comprising, in amino- to carboxyl-terminal order, the albumin moiety, a linker, a first TRAIL monomer, an inter-monomer linker, a second TRAIL monomer, a second inter-monomer linker, and a third TRAIL monomer.

### v. Bispecific Fusion Polypeptides

Also provided are bispecific antibody fusions. In one embodiment, the TRAIL polypeptide is fused to the c-terminus of a heavy chain of a bispecific antibody. Bispecific antibodies herein include at least two binding specificities for the same or different proteins which preferably bind non-overlapping or non-competing epitopes. Such bispecific antibodies can include additional binding specificities, e.g., a third protein binding specificity for another antigen, such as the product of an oncogene. Bispecific antibodies can be prepared as full length antibodies or antibody fragments (e.g. F(ab')₂ bispecific antibodies).

### E. Methods of Producing TRAIL Fusion Polypeptides

The TRAIL fusion proteins described herein can be produced by standard recombinant techniques. Methods for recombinant production are widely known in the state of the art and comprise protein expression in prokaryotic and eukaryotic cells with subsequent isolation of the antibody and usually purification to a pharmaceutically acceptable purity. For the expression of the binding proteins in a host cell, nucleic acids encoding the respective polypeptides are inserted into expression vectors by standard methods. Expression is performed in appropriate prokaryotic or eukaryotic host cells (such as CHO cells, NSO cells, SP2/0 cells, HEK293 cells, COS cells, PER.C6 cells, yeast, or E. coli cells), and the binding protein is recovered from the cells (supernatant or cells after lysis). General methods for recombinant production of antibodies are well-known in the state of the art and described, for example, in the review articles of Makrides, S.C., Protein Expr. Purif 17 183-202 (1999); Geisse, S., et al, Protein Expr. Purif. 8 271-282 (1996); Kaufman, R.J., MoI. Biotechnol. 16 151-161 (2000); Werner, R.G., Drug Res. 48 870-880 (1998).

The polypeptides may be suitably separated from the culture medium by conventional purification procedures. Purification can be performed in order to eliminate cellular components or other contaminants, e.g. other cellular nucleic acids or proteins, by standard techniques, including alkaline/SDS treatment, CsC1 banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxylmethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M.A. Appl. Biochem. Biotech. 75 93-102 (1998)). DNA and RNA encoding the polypeptides are readily isolated and sequenced using conventional procedures.

### F. Patient Populations

Provided herein are methods for treating cancer in a human patient and for selecting patients to be so treated based on particular levels of biomarkers, such as DR4 (TNFRSF10A) or cIAP1 (BIRC2). In one embodiment, the cancer is selected from the group consisting of colorectal cancer, non-small cell lung cancer (NSCLC), renal cell carcinoma (RCC), melanoma (e.g., cutaneous or intraocular malignant melanoma), serous ovarian carcinoma, liver cancer, bone cancer, pancreatic cancer, skin cancer, cancer of the head or neck, breast cancer, lung cancer, uterine cancer, colon cancer, rectal cancer, cancer of the anal region, esophageal cancer, gastric cancer, gastro-esophageal junction cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), spinal axis tumor, glioma, pituitary adenoma, Kaposi's sarcoma, epidermoid cancer, squamous cell cancer, and mesothelioma. The methods are also applicable to treatment of metastatic cancers.

In one embodiment, the patient has evidence of recurrent or persistent disease following primary chemotherapy. In another embodiment, the patient has had at least one prior platinum based chemotherapy regimen for management of primary or recurrent disease. In another embodiment, the patient has a cancer that is platinum-resistant or refractory. In another embodiment, the patient has evidence of recurrent or persistent disease following a) primary treatment or b) an adjuvant treatment.

In another embodiment, the patient has an advanced cancer. In one embodiment, the term “advanced” cancer denotes a cancer above Stage II. In another, “advanced” refers to a stage of disease where chemotherapy is typically recommended, which is any one of the following: 1. in the setting of recurrent disease: any stage or grade; 2. stage IC or higher, any grade; 3. stage IA or IB, grade 2 or 3; or 4. in the setting of incomplete surgery or suspected residual disease after surgery (where further surgery cannot be performed): any stage or grade.

### G. Outcomes

The efficacy of the treatment methods provided herein can be assessed using any suitable means. In one embodiment, the treatment produces at least one therapeutic effect selected from the group consisting of reduction in growth rate of tumor, reduction in size of tumor, reduction in number of metastatic lesions over time, increase in duration of progression-free survival, and increase in overall response rate. The method provided herein, may inhibit tumor growth by at least about 10%, for example, at least about 20%, at least about 30%, at least about 40%, at least about 50%, at least about 60%, at least about 70%, at least about 80%, at least about 90%, at least about 99%, or 100%.

In one embodiment, the treatment methods described herein result in greater than 50% cancer cell death. In another embodiment, the treatment results in greater than 60% cancer cell death, greater than 65% cancer cell death, greater than 70% cancer cell death, greater than 75% cancer cell death, greater than 80% cancer cell death, greater than 85% cancer cell death, greater than 90% cancer cell death, greater than 95% cancer cell death, or 100% cancer cell death.

With respect to target lesions, responses to therapy may include:

Complete Response (CR): Disappearance of all target lesions. Any pathological lymph nodes (whether target or non-target) must have reduction in short axis to <10 mm;

Partial Response (PR): At least a 30% decrease in the sum of the diameters of target lesions, taking as reference the baseline sum diameters;

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression); and

Stable Disease (SD): Neither sufficient shrinkage to qualify for PR, nor sufficient increase to qualify for PD, taking as reference the smallest sum diameters while on study. (Note: a change of 20% or less that does not increase the sum of the diameters by 5 mm or more is coded as stable disease). To be assigned a status of stable disease, measurements must have met the stable disease criteria at least once after study entry at a minimum interval of 6 weeks.

With respect to non-target lesions, responses to therapy may include:

Complete Response (CR): Disappearance of all non-target lesions and normalization of tumor marker level. All lymph nodes must be non-pathological in size (<10 mm short axis). If tumor markers are initially above the upper normal limit, they must normalize for a patient to be considered in complete clinical response;

Non-CR/Non-PD: Persistence of one or more non-target lesion(s) and/or maintenance of tumor marker level above the normal limits; and

Progressive Disease (PD): Appearance of one or more new lesions and/or unequivocal progression of existing non-target lesions. Unequivocal progression should not normally trump target lesion status. It must be representative of overall disease status change, not a single lesion increase.

In exemplary outcomes, patients treated according to the methods disclosed herein may experience improvement in at least one sign of a cancer.

In one embodiment, the patient so treated exhibits CR, PR, or SD.

In another embodiment, the patient so treated experiences tumor shrinkage and/or decrease in growth rate, i.e., suppression of tumor growth. In yet another embodiment, one or more of the following can occur: the number of cancer cells is reduced; tumor size is reduced; cancer cell infiltration into peripheral organs is inhibited, retarded, slowed, or stopped; tumor metastasis is slowed or inhibited; tumor growth is inhibited; recurrence of tumor is prevented or delayed; or one or more of the symptoms associated with cancer is relieved to some extent.

In other embodiments, such improvement is measured by a reduction in the quantity and/or size of measurable tumor lesions. Measurable lesions are defined as those that can be accurately measured in at least one dimension (longest diameter is to be recorded) as >10 mm by either or both of CT scan (CT scan slice thickness no greater than 5 mm) and caliper measurement via clinical exam, or as >20 mm by chest X-ray. The size of non-target lesions, e.g., pathological lymph nodes, can also be measured for improvement. In one embodiment, lesions can be measured on chest x-rays or CT or MRI outputs.

In other embodiments, cytology or histology can be used to evaluate responsiveness to a therapy. The cytological confirmation of the neoplastic origin of any effusion that appears or worsens during treatment when the measurable tumor has met criteria for response or stable disease can be considered to differentiate between response or stable disease (an effusion may be a side effect of the treatment) and progressive disease.

### H. Kits and Articles of Manufacture

Further provided are kits containing a TRAIL-based therapeutic or death receptor agonist and instructions for use according to the methods described herein. Kits typically include a packaged combination of reagents in predetermined amounts with instructions and a label indicating the intended use of the contents of the kit. The term label or instruction includes any writing, or recorded material supplied on or with the kit, or which otherwise accompanies the kit at any time during its manufacture, transport, sale or use. It can be in the form prescribed by a governmental agency regulating the manufacture, use or sale of pharmaceuticals or biological products, which notice reflects approval by the agency of the manufacture, use or sale for administration to a human or for veterinary use. The label or instruction can also encompass advertising leaflets and brochures, packaging materials, and audio or video instructions.

For example, in some embodiments, the kit contains the TRAIL-based therapeutic or death receptor agonist in suitable containers and instructions for administration in accordance with the treatment regimens described herein. In some embodiments, the kit further comprises an additional antineoplastic agent. In some embodiments, the TRAIL-based therapeutic or death receptor agonist are provided in suitable containers as a dosage unit for administration. Suitable containers include, for example, bottles, vials, syringes, and test tubes. The containers may be formed from a variety of materials such as glass or plastic.

The following examples are merely illustrative and should not be construed as limiting the scope of this disclosure in any way as many variations and equivalents will become apparent to those skilled in the art upon reading the present disclosure.

All patents, patent applications and publications cited herein are incorporated herein by reference in their entireties.

### EXAMPLES

### Example 1: Logic Model Used to Derive Predictive Biomarker in CRC

Gene expression data were used to train logic models that predict response to cross-linked recombinant human TRAIL (rhTRAIL). For the training set, publicly available data were used: gene expression data from the Cancer Cell Line Encyclopedia (CCLE) and cell viability changes by cross-linked rhTRAIL on a panel of CRC cell lines. AND and OR functions were applied to all possible pairs of genes from the BIOCARTA Death Pathway gene list to create logic model scores. A two-gene biomarker was discovered, TNFRSF10A AND (NOT BIRC2), that correlated well with maximum inhibition by cross-linked rhTRAIL, and bootstrapping predicted that this would be statistically significant.

### Materials and Methods

### Cell Lines

Colorectal cell lines were purchased/obtained from American Type Culture Collection (ATCC, Manassas, Va., USA), National Cancer Institute (NCI, Frederick, Md., USA), Sigma-Aldrich (St. Louis, Mo., USA), Korean Cell Line Bank (KCLB, Seoul, Korea), National Institute of Biomedical Innovation (NIBIO, Osaka, Japan), or Japanese Collection of Research Bioresources Cell Bank (JCRB, Osaka, Japan), as indicated in Tables 1A and 1B. All cells were grown at 37° C. in 5% CO2 except for SW1417 cells, which were maintained in an incubator with 100% air atmosphere. The culture conditions of all cell lines are provided in Tables 1A and 1B. All growth media were supplemented with 10% heat-inactivated fetal bovine serum (HI FBS, Gibco Life Technologies, and Grand Island, N.Y., USA), 100 units/ml penicillin, and 100 μg/ml streptomycin (Gibco Life Technologies). For the C2BBe-1 cell line, 0.01 mg/mL human transferrin (Sigma, Cat # T8158) was added to the growth media along with other supplements. Cell culture media were purchased from Gibco Life Technologies, except for EMEM media which was obtained from ATCC.

### Cell Viability Assay

Colorectal cell lines were cultured in complete media to sub-confluency. The cells were detached with trypsin (Gibco Life Technologies, Grand Island, N.Y., USA), washed once with PBS pH 7.4 (Gibco Life Technologies,) and suspended in complete fresh growth media. The cells were seeded in 96-well plates at optimal density as shown in Tables 1A and 1B, and allowed to recover overnight at 37 ° C. Cells were then treated with increasing concentrations (0-10 nM) of recombinant human TRAIL (rhTRAIL, R&D Systems, Minneapolis, Minn., USA). At 24 hours post treatment, viability was determined by measuring the amount of cellular ATP using CellTiter-Glo® Luminescent Assay (CTG, Promega Life Sciences, Madison, Wis., USA) according to manufacturer's protocols. Data were obtained from at least two independent studies and luminescence was normalized to untreated controls. Dose-response curves were generated and Aₘₐₓ values were calculated using MATLAB (Natick, Mass., USA).

TABLE 1A
Cell lines used for training the logic model

| | Source | | Optimal |
|---|---|---|---|
| | of Cell | | Seeding |
| Cell line | Line | Media Condition | Density |
| C2BBe-1 | ATCC | DMEM + 10% FBS + Pen | 10000 |
| | | Strep + Transferrin | |
| Colo 201 | ATCC | RPMI + 10% FBS + Pen Strep | 10000 |
| HCT15 | NCI | RPMI + 10% FBS + Pen Strep | 10000 |
| HCT116 | NCI | RPMI + 10% FBS + Pen Strep | 10000 |
| HT29 | ATCC | RPMI + 10% FBS + Pen Strep | 10000 |
| KM12 | NCI | RPMI + 10% FBS + Pen Strep | 10000 |
| LOVO | ATCC | DMEM/F12 (1:1) + 10% FBS + | 10000 |
| | | Pen Strep | |
| LS180 | ATCC | EMEM + 10% FBS + Pen Strep | 10000 |
| MDST8 | Sigma | DMEM + 10% FBS + Pen Strep | 10000 |
| RKO | ATCC | EMEM + 10% FBS + Pen Strep | 10000 |
| SW620 | NCI | RPMI + 10% FBS + Pen Strep | 10000 |

TABLE 1B
Cell lines used for testing the logic model

| | Source | | Optimal |
|---|---|---|---|
| | of Cell | | Seeding |
| Cell line | Line | Media Condition | Density |
| CCK81 | JCRB | EMEM + 10% FBS + Pen Strep | 10000 |
| GP2D | Sigma | RPMI + 10% FBS + Pen Strep | 20000 |
| LS123 | ATCC | EMEM + 10% FBS + Pen Strep | 20000 |
| LS411N | ATCC | RPMI + 10% FBS + Pen Strep | 10000 |
| NCIH747 | ATCC | RPMI + 10% FBS + Pen Strep | 10000 |
| RCM1 | NIBIO | RPMI + 10% FBS + Pen Strep | 20000 |
| SNU175 | KCLB | RPMI + 10% FBS + Pen Strep | 20000 |
| SNUC2A | KCLB | RPMI + 10% FBS + Pen Strep | 20000 |
| SW1417 | ATCC | Leibovitz's L-15 + 10% FBS + | 5000 |
| | | Pen Strep (No CO₂) | |

### Processing RNAseq Data

The CCLE consortium has published raw RNA-Seq data for 933 cell lines. These data were processed using the RNA-Seq quantification program kallisto (N. L. Bray, H. Pimentel, P. Melsted, L. Pachter, A. Rxiv (2015)). This program outputs gene abundance for each cell line, and these data were then upper quantile normalized. Because biological noise is often proportional to signal, the data were then loge-normalized so that results would not be skewed by samples with high mRNA expression of particular genes. Finally, to use the data in logic models, the expression for each gene was normalized to a value between 0 and 1 inclusive. Expression for each gene was normalized by the 95^{th} and 5^{th} percentile values for that gene across all 933 CCLE samples.

The genes that were included in subsequent analyses are those that are in the curated, publicly available BIOCARTA Death Pathway (see Table 2).

TABLE 2
Genes in the BIOCARTA Death Pathway

| | | | |
|---|---|---|---|
| APAF1 | CASP7 | FADD | SPTAN1 |
| BCL2 | CASP8 | GAS2 | TNFRSF10A |
| BID | CASP9 | LMNA | TNFRSF10B |
| BIRC2 | CFLAR | MAP3K14 | TNFRSF25 |
| BIRC3 | CHUK | NFKB1 | TNFSF10 |
| CASP10 | CYCS | NFKBIA | TNFSF12 |
| CASP3 | DFFA | RELA | TRADD |
| CASP6 | DFFB | RIPK1 | TRAF2 |
| | | | XIAP |

### Building Logic Models

When considering continuous variables X and Y ranging between 0 and 1 inclusive, logic gates “AND”, “OR” and “NOT” can be described using the following equations:

NOT X=1−X

X AND Y=X*Y

X OR Y=NOT((NOT X) AND (NOT Y))=1−((1−X)*(1−Y))

Logic models were created for all pairs of genes and their NOT values for all CRC cell lines in the CCLE. The percent of cells remaining was correlated with logic model scores. Models were considered non-random if they correlated with actual viability at least as well as the correlation with 95% of scrambled outputs.

### Choosing the Best Models

A publicly available dataset reporting cell line response to cross-linked rhTRAIL was used as a training set to choose the most predictive logic models (P. M. Nair et al., Proceedings of the National Academy of Sciences 112, 5679-5684 (2015)). Cell viability data from CCLE cell lines derived from the colon and cecum was correlated with logic model scores. Bootstrapping was performed by correlating scrambled viability data with logic model scores. Logic models were considered to be non-random if they correlated with actual viability at least as well as the correlation with 95% of the scrambled outputs.

### Testing the Models with rhTRAIL

The biomarker hypothesis was then tested with rhTRAIL. Fourteen cell lines that were in the original dataset were exposed to rhTRAIL in a dose-dependent manner and cell viability was measured using a CellTiter Glo® (CTG) assay.

Before the hypothesis was validated on new cell lines, a linear model was fit to predict cell viability based on logic model scores from the rhTRAIL dataset. Logic model scores were calculated for nine additional CRC cell lines based on their RNA-Seq values from the CCLE (as was done for the training set) and cell viability was predicted and then compared against the median of actual minimum viability for each cell line.

### Results

### Biomarker Prediction

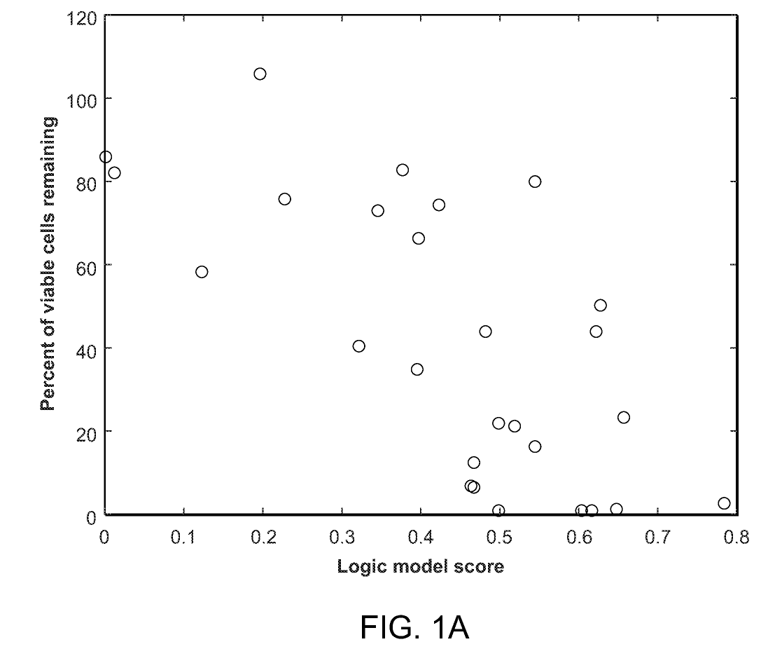

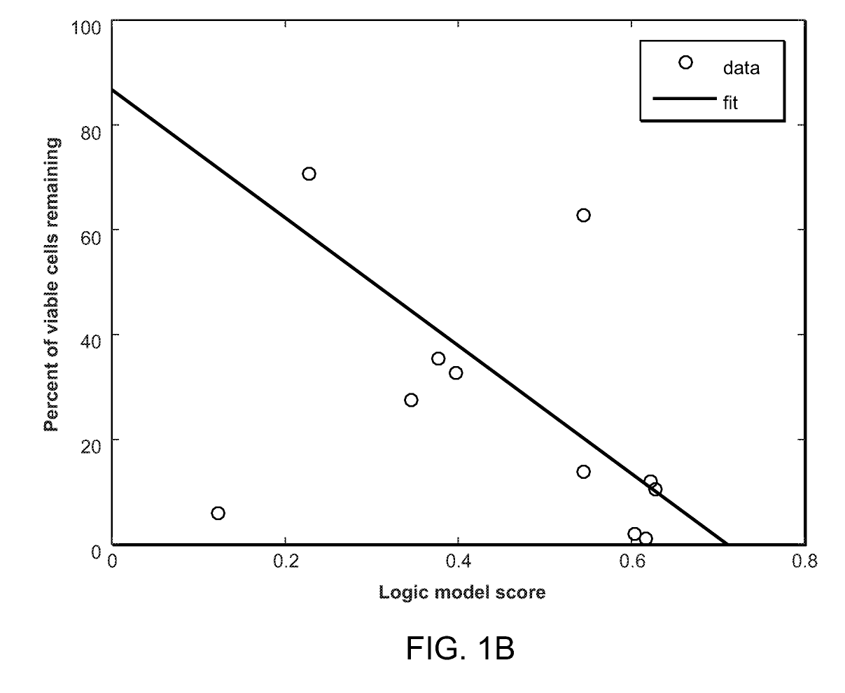

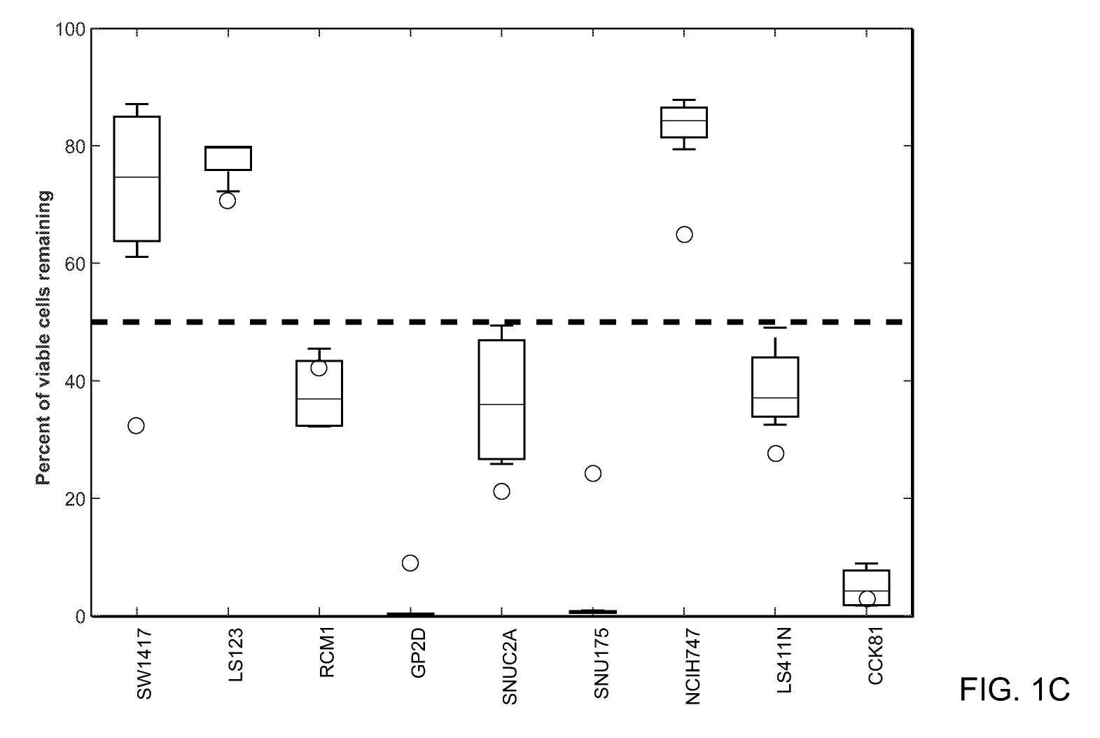

One of the best predictors of cell viability was found to be DR4 (TNFRSF10A). TNFSFR10A is the gene encoding Death Receptor 4, and BIRC2 is the gene encoding the anti-apoptotic protein cIAP1. The higher the output of this logic function, the more the cross-linked rhTRAIL was able to reduce cell viability. The Pearson correlation between the logic model score and cell viability was −0.69. This was calculated using 27 samples. A graph of the logic model fit vs. the percent of viable cells remaining is shown in FIG. 1A.

Bootstrapping analysis showed that the correlation between the predictors resulting from the logic function TNFRSF10A AND (NOT BIRC2) and the actual response was better than the correlation between the best predictors from the scrambled dataset 974 out of 1000 times (i.e., p=0.026). This indicated that the biomarker data were not random. Thus, the model proposes that DR4 and cIAP1 are the most sensitive nodes in the TRAIL signaling pathway. This model suggests that, if DR4 is not present, rhTRAIL cannot transmit its signal from outside the cell. If cIAP1 levels are too high, the anti-apoptotic signal will overpower apoptotic cues from TRAIL, thus requiring a balance of high DR4 and low cIAP1 for rhTRAIL to induce apoptosis.

The same trends that were observed in the original publicly available dataset held true in the assay with a smaller set of cell lines that was exposed to rhTRAIL: the median of maximum inhibition of cell viability for each cell line was found to correlate with the logic score from the function TNFRSF10A AND (NOT BIRC2). Table 3 summarizes CTG data for the model training set, using 11 CRC cell lines that overlap with the original publicly available dataset that were used to fit the regression model to predict cell viability from logic model scores.

TABLE 3
Minimum Percent Cell Viability in the Presence of rhTRAIL

| Cell Line | Rep 1 | Rep 2 | Rep 3 | Rep 4 | Rep 5 | Rep 6 | Rep 7 |
|---|---|---|---|---|---|---|---|
| C2BBE1 | 32.414095 | 38.97575 | 32.65526 | 41.95817 | 39.74466 | 35.39979 | 31.18505 |
| COLO201 | 0.8705625 | 0.824415 | 1.317418 | 1.490751 | 0.950274 | 1.032271 | 0.191561 |
| HCT116 | 13.879338 | 11.97673 | 9.69697 | 13.47826 | 16.90531 | 13.91236 | 14.65688 |
| HCT15 | 1.555543 | 1.891978 | 2.441386 | 2.59009 | 4.738243 | 1.398022 | 1.444062 |
| HT29 | 32.821457 | 50.56871 | 32.18778 | 51.43426 | 53.31691 | 32.58101 | 24.66045 |
| KM12 | 22.100375 | 44.98798 | 27.32282 | 36.65409 | 48.91292 | 27.40696 | 22.2437 |
| LOVO | 4.4235925 | 15.53081 | 15.44929 | 12.07796 | 19.03836 | 3.733253 | 3.912716 |
| LS180 | 10.460083 | 10.25373 | 13.41075 | 11.69129 | 16.41002 | 3.17609 | 5.588585 |
| MDST8 | 6.0480784 | 6.194176 | 8.81882 | 5.119098 | 5.997491 | 6.381583 | 2.878234 |
| RKO | 69.030462 | 70.69706 | 81.02936 | 78.09553 | 72.49558 | 66.87089 | 66.17501 |
| SW620 | 63.95638 | 57.24391 | 67.24857 | 61.04062 | 59.77765 | 67.39022 | 62.61596 |

A graph of the logic model output, the percent cell viability and the fit is shown in FIG. 1B. A regression model (red line) is fit to predict cell viability in response to 10 nM rhTRAIL as a function of the logic model score. Regression was performed using data from 10 out of the 11 cell lines. MDST8 (logic model score=0.14 and cell viability=6%) was excluded from the fit to create a predictive model because it is clearly an outlier. The regression model is: percent of viable cells remaining=86.7-122.0*(logic model score).

Nine additional CRC cell lines that were not part of the original publicly available dataset were then exposed to rhTRAIL and cell viability was measured. These cell lines express varying levels of DR4 and cIAP1, based on CCLE data.

Table 4 summarizes CTG data from the test set using the nine additional CRC cell lines that were exposed to rhTRAIL to validate the biomarker hypothesis.

TABLE 4
Minimum Percent Cell Viability in the presence of rhTRAIL

| Cell Line | Rep 1 | Rep 2 | Rep 3 | Rep 4 |
|---|---|---|---|---|
| CCK81 | 1.529686285 | 1.374531 | 5.771396 | 7.869524 |
| GP2D | 0.16305234 | 0.241734 | 0.342833 | 0.372376 |
| LS123 | 67.20183486 | 70.06956 | 75.84583 | 68.95748 |
| LS411N | 31.07165504 | 32.54284 | 38.96952 | 37.78724 |
| NCIH747 | 79.04129962 | 76.26916 | 82.04991 | 77.21605 |
| RCM1 | 29.65709729 | 32.19447 | 43.95882 | 41.24717 |
| SNU175 | 0.929614874 | 0.683234 | 0.445765 | 0.418994 |
| SNUC2A | 45.30132789 | 38.03599 | 23.17659 | 27.46143 |
| SW1417 | 76.70129567 | 76.93096 | 58.28165 | 65.0076 |

Table 5 summarizes normalized DR4 and cIAP1 levels from the test set using nine additional CRC cell lines that were exposed to rhTRAIL to validate the biomarker hypothesis.

TABLE 5
Normalized DR4 and
cIAP1 levels in the test set

| Cell line | DR4 | cIAP1 |
|---|---|---|
| SW1417 | 0.64 | 0.31 |
| LS123 | 0.69 | 0.81 |
| RCM1 | 0.79 | 0.54 |
| GP2D | 0.80 | 0.20 |
| SNUC2A | 1.00 | 0.46 |
| SNU175 | 0.79 | 0.35 |
| NCIH747 | 0.74 | 0.76 |
| LS411N | 0.73 | 0.33 |
| CCK81 | 0.81 | 0.15 |

The cell lines were exposed in a dose-dependent manner to rhTRAIL (4 replicates/cell line). Minimum cell viability was recorded.

The logic model predicted the response of eight out of the nine additional cell lines correctly as either responders or non-responders to rhTRAIL, using a 50% inhibition cut off (for example, a cell line with greater than 50% cell death after treatment is classified as a responder, and a cell line with less than 50% cell death after treatment is a non-responder), and predicted actual viability within 10% of at least one measured value for six of the cell lines. The predictions vs. the actual cell viability are shown in FIG. 1C. Taken together, these results demonstrate that patients whose tumors have high levels of DR4 and low levels of cIAP1 are likely to respond favorably to treatment with a death receptor agonist.

### Example 2: Analysis of Cancer Genomic Information

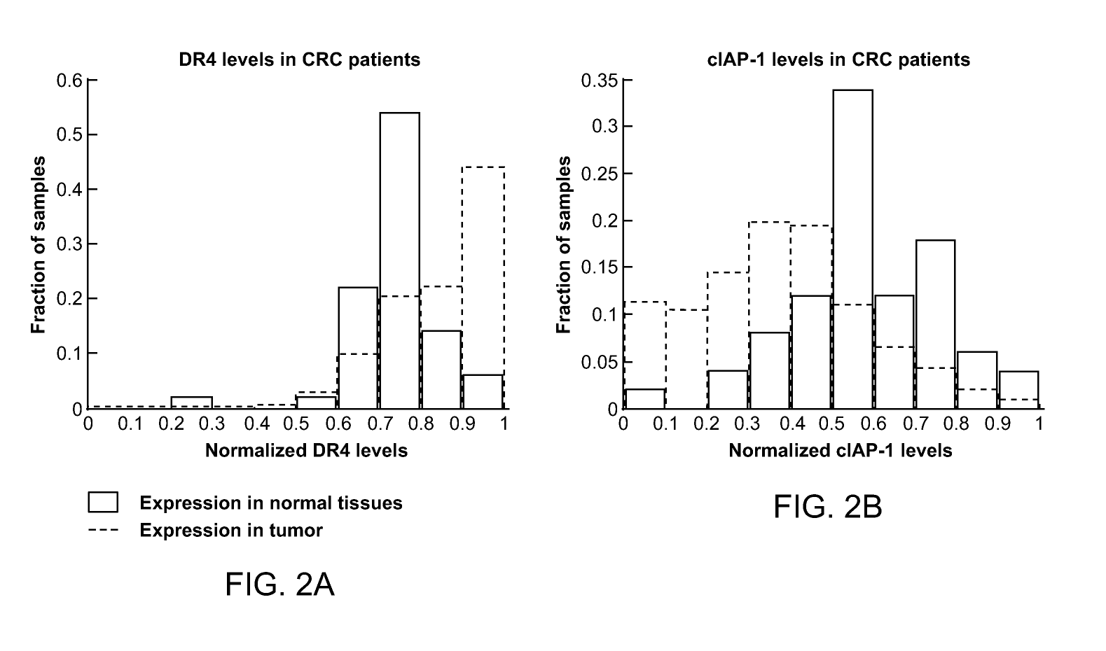

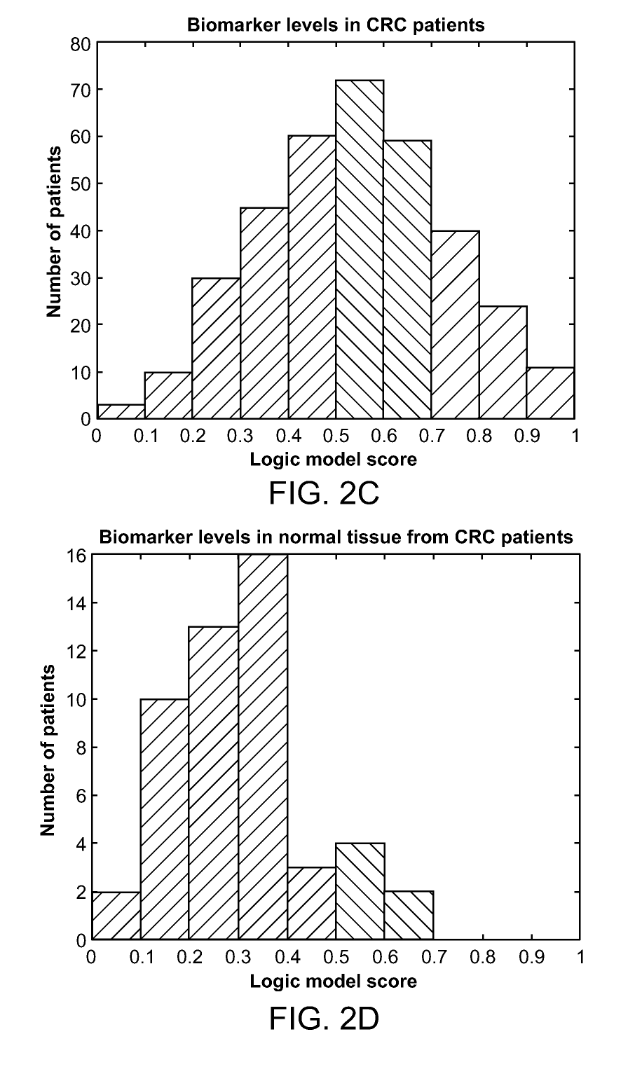

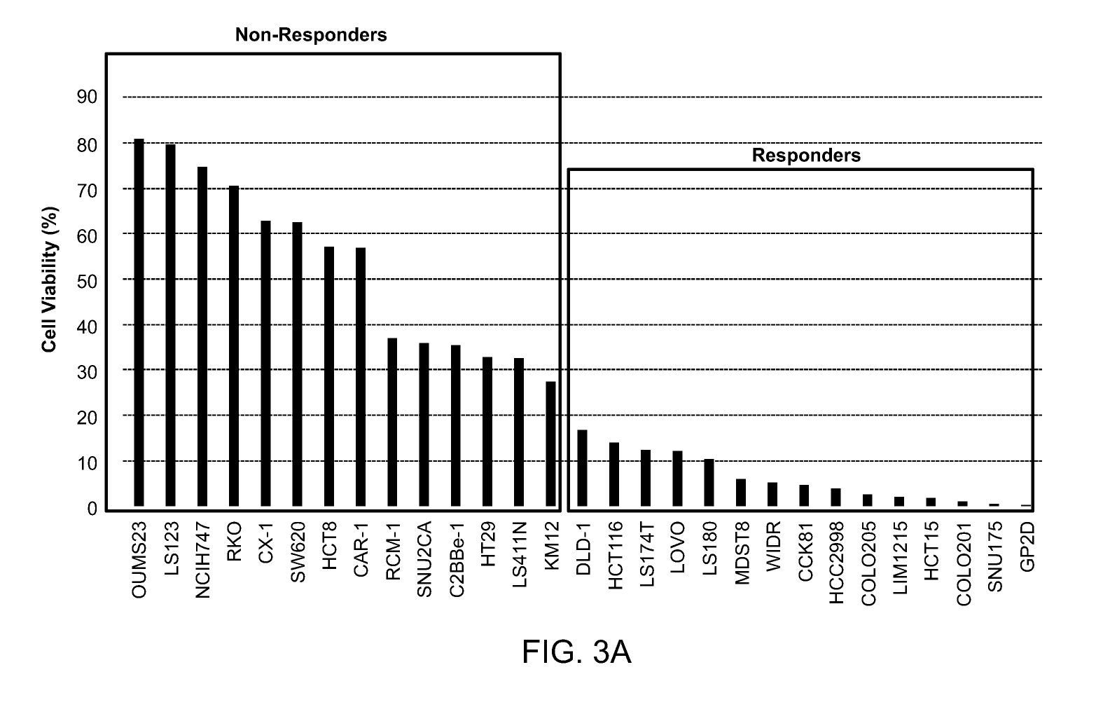

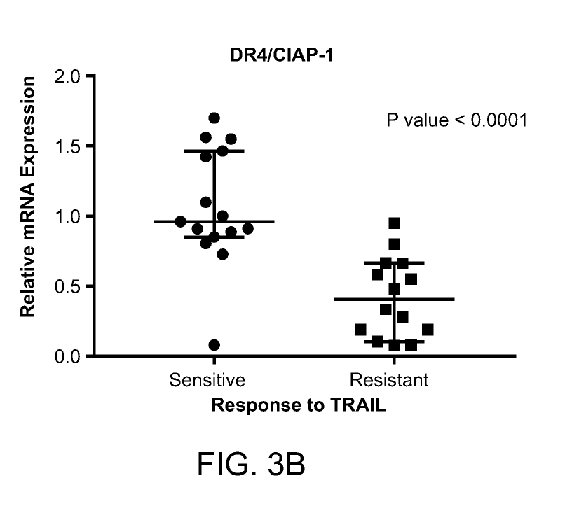

Analysis was performed on colorectal data from The Cancer Genome Atlas (TCGA) to predict the fraction of the population that would respond to TRAIL. Briefly, the gene expression of each sample was normalized across all samples in all indications. FIG. 2A shows a histogram of normalized DR4 levels in CRC tumor samples. The distribution clearly skews to the right, showing that most CRC tumor samples have high levels of DR4. FIG. 2B shows a histogram of normalized cIAP1 levels in CRC tumor samples, which skew to the left. FIG. 2C displays the distribution of biomarker scores across CRC tumor samples, based on normalized DR4 and cIAP1 levels. It should be noted that 58% of samples have a biomarker score greater than 0.5, which corresponds to a predicted 75% reduction in cell viability. 21% of samples have a biomarker score greater than 0.7, which is predicted to completely eradicate tumor cells.

Similar analysis was performed on normal colorectal tissue samples from TCGA. A histogram of the biomarker scores across normal colorectal tissue samples is shown in FIG. 2D. Noticeably, only 12% of samples have a score greater than 0.5, and no samples have a score greater than 0.7, suggesting that TRAIL treatment may not be toxic to normal colorectal tissue.

### Example 3: DR4/cIAP1 mRNA Expression Ratio Correlates with Response to TRAIL-Based Therapy

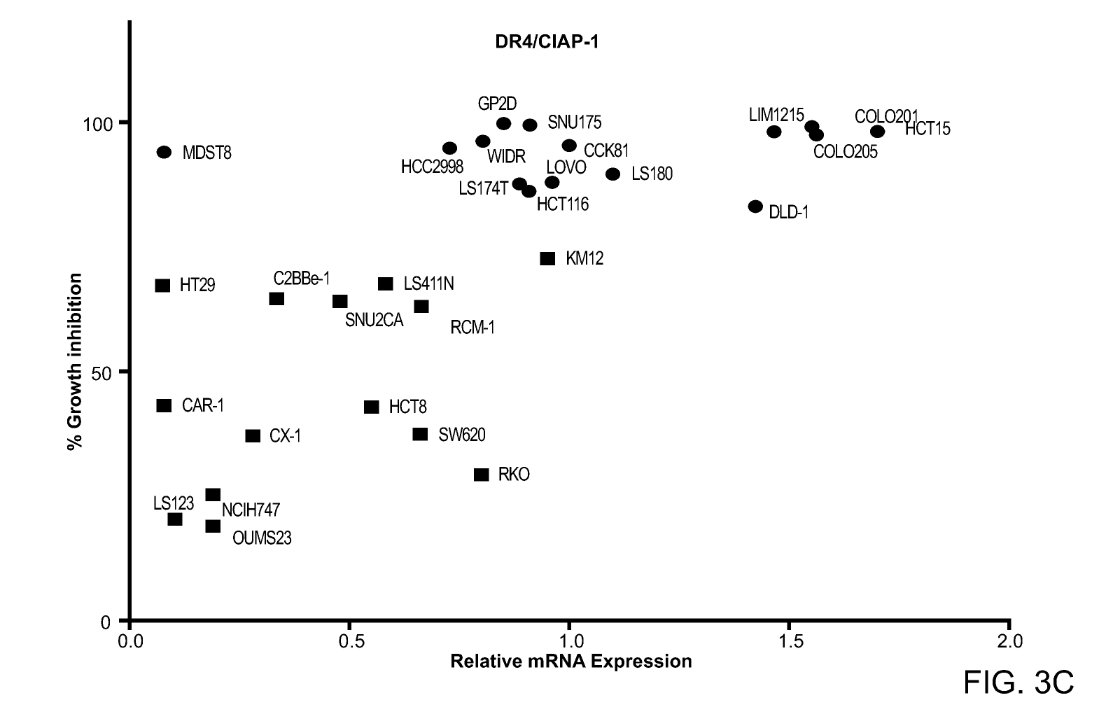

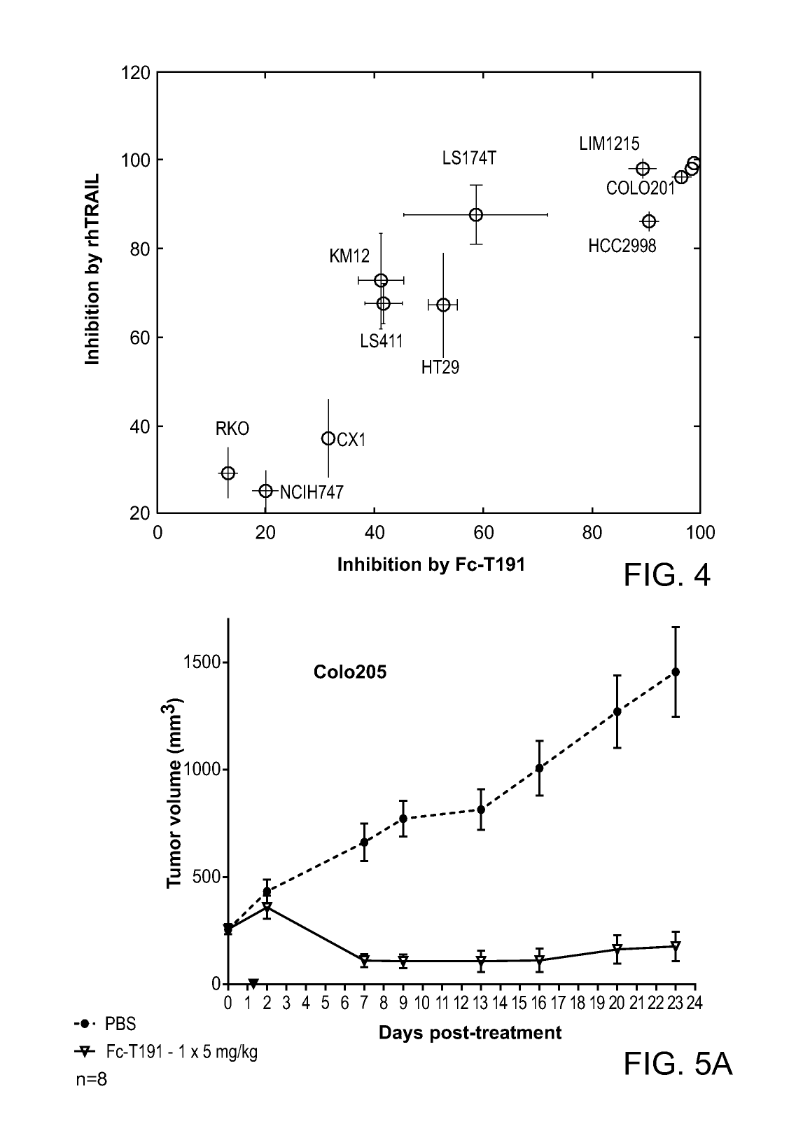

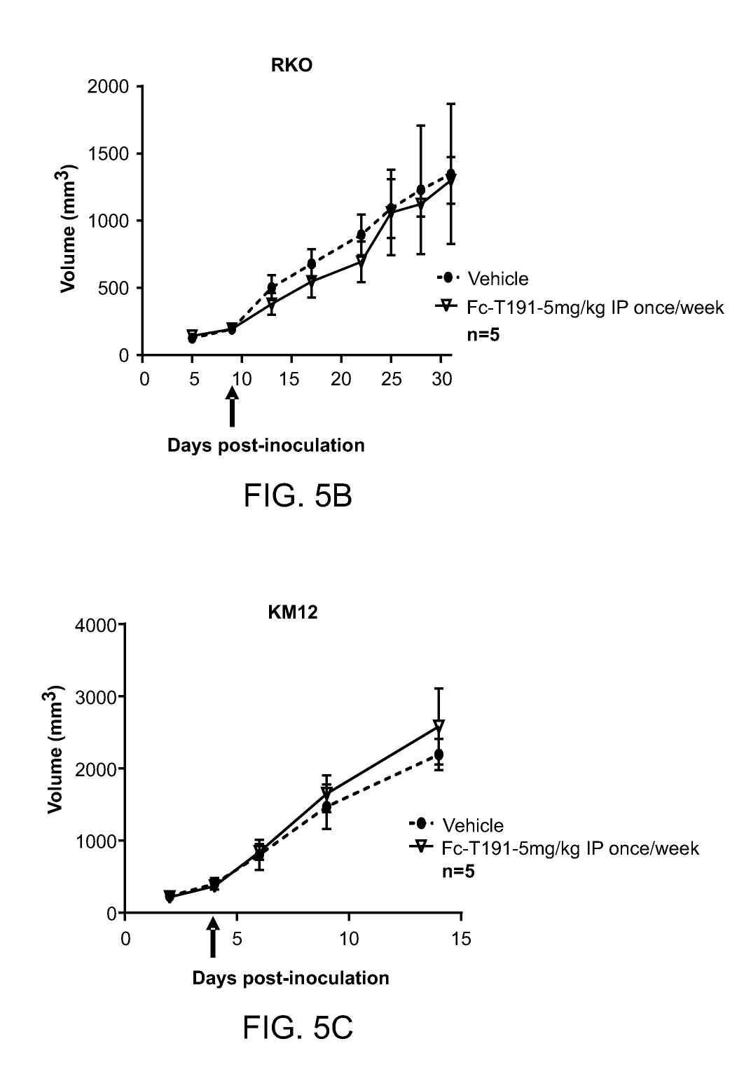

Colorectal cell lines were treated with different concentrations of rhTRAIL (0-10 nM) for 24 hours. The anti-tumor activities (viability) were assessed at 10 nM (Cₘₐₓ) using CellTiter-Glo luminescence assays. FIG. 3A shows percent viability of indicated cell lines following treatment. Cell lines with 80% loss in viability were classified as sensitive cell lines and the remaining as resistant cell lines. Sensitive cell lines showed significantly higher DR4/cIAP1 mRNA expression ratio as compared to resistant cell lines (FIGS. 1B and 1C).

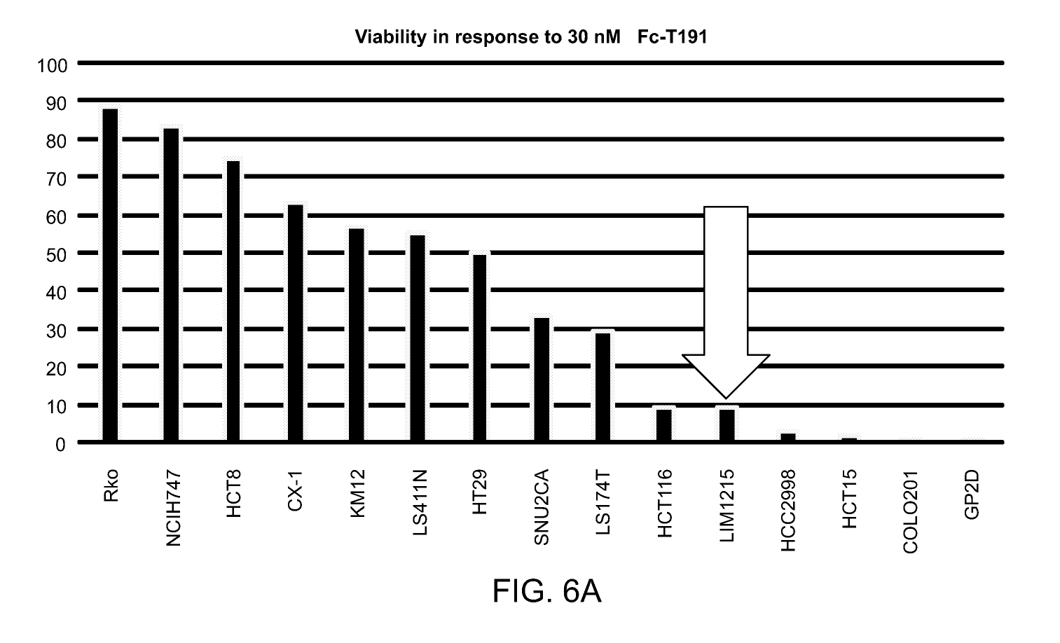

Current TRAIL agonists (i.e., rhTRAIL) have poor pharmacokinetics properties and/or suffer from low potency. However, Fc-TRAIL (e.g., Fc-T191), is a single fusion polypeptide consisting of an Fc region followed by three successive TRAIL monomers targeting both DR4 and DRS, which shows superior terminal half-life in mice PK studies. Fc-T191 shows comparable activity to rhTRAIL with a correlation coefficient=0.92. Thus most of the CRC cell lines that responded well to rhTRAIL, also responded well to Fc-T191 (FIG. 4).

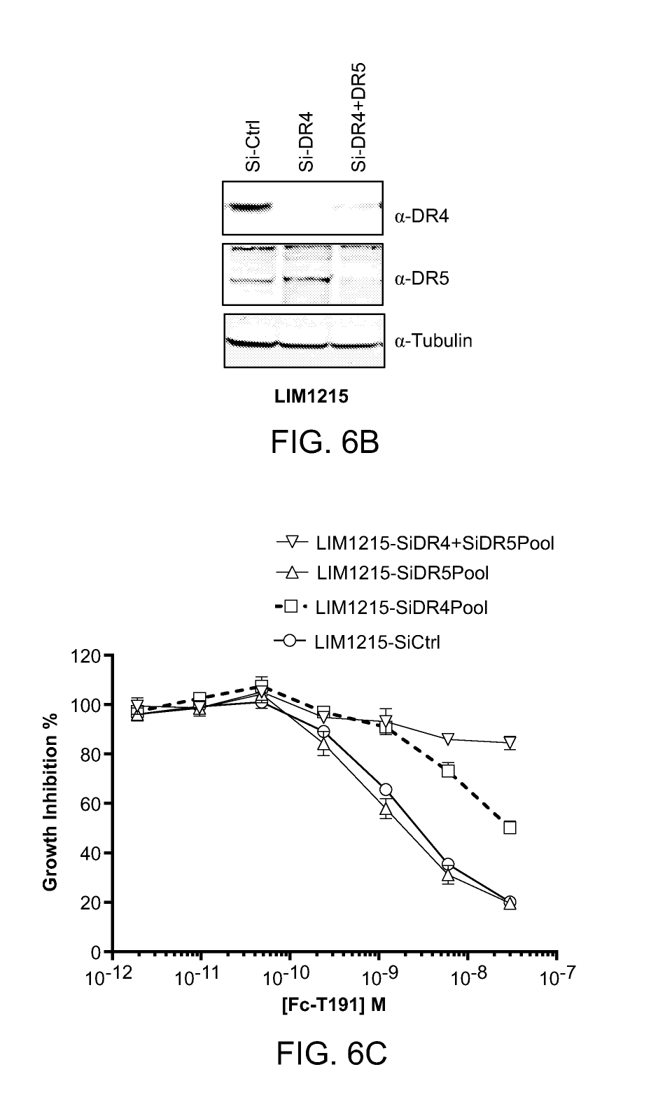

The Colo205 cell line was used to test the biomarker hypothesis in vivo because it has high DR4 (TNFRSF10A) and low cIAP1 (BIRC2) expression, i.e., a high DR4/cIAP1 ratio. Treatment with Fc-T191 (10 mg/kg IP) alone inhibited tumor growth significantly (FIG. 5A). Notably, the growth of RKO and KM12 cell lines with relative low DR4/cIAP1 ratios respectively, were largely unaffected by the treatment of Fc-T191 (FIGS. 5B and 5C). These results suggested that high DR4 (TNFRSF10A) and low cIAP1 (BIRC2) levels indicate that TRAIL therapy will be successful.

### Example 4: Knockdown of Death Receptors and cIAP1 Alters the Response of CRC Cell Lines to TRAIL Indicating a Mechanistic Relationship Between the Two-Gene Biomarker and the Response

Since DR4 and cIAP1 were identified as a potential biomarker signature using computational modeling, the functional role of DR4 or DR5 in response to Fc-T191 treatment in LIM-1215 and HCT116 cells was tested.

### Materials and Methods

### RNA Silencing/Transfection

SiRNA(s) targeting human DR4, DR5, and cIAP1 genes and universal scrambled siRNAs were obtained from IDT (Integrated DNA Technologies, Coralville, Iowa, USA) and the siRNA sequences were as follows:

siDR4-Duplex-1
(SEQ ID NO: 41)
GGAACUUUCCGGAAUGACAAUUCTG
siDR4-Duplex-2
(SEQ ID NO: 42)
GGACAAUGCUCACAACGAGAUUCTG
siDR4-Duplex-3
(SEQ ID NO: 43)
CAGAAUCUCGUUGUGAGCAUUGUCCUC
siDR5-Duplex-1
(SEQ ID NO: 44)
GUCACAUGACCGGUACUGGAAGAAA
siDR5-Duplex-2
(SEQ ID NO: 45)
UACCUUCUAGAUACAUGAACUUUCCAG
siDR5-Duplex-3
(SEQ ID NO: 46)
UCUGAGACAGUGCUUCGAUGACUTT
SiCIAP-1 Duplex-1
(SEQ ID NO: 47)
AAUGAUGCAUAACAAGCAGUGACACUA
SiCIAP-1 Duplex-2
(SEQ ID NO: 48)
AUACCUUUACAAGCGAGAGAACUGA
SiCIAP-1 Duplex-3
(SEQ ID NO: 49)
UUGGAAUAUACUUCAUAUUCUUAUCCA

HCT116, LIM1215 cells (800,000) were reverse-transfected in 6 well plates with SiRNA pools (30pmo1 total for DR4, DRS and cIAP1 alone or 15pmol for DR4- and DR5 combined) using 9ul of RNAi-Max (Invitrogen) transfection reagent in the absence of antibiotics. Cells were washed with PBS and cultured for 2 days after transfection, trypsinized and replated in 96 well plates (10,000 cells/well) and treated with increasing concentration of Fc-T191 for 24 hours (0-30 uM doses) in triplicates. CTG readouts were measured after 24 hours. Cellular lysates were prepared at the same time to validate knockdowns using western blot analysis.

### Western Blotting Analysis

48 hours after transfection cells were washed with ice cold Dulbecco's Phosphate Buffer Saline (PBS), pH7.4 (Gibco), trypsinized with 0.25% trypsin (Gibco), and collected into 15 ml tubes. Cells were pelleted and washed once with ice cold PBS before addition of 250 μl of lysis buffer (RIPA Lysis and Extraction Buffer (Thermo Scientific)+Protease Inhibitor Cocktail (Sigma), Phosphatase Inhibitor Cocktail 2 (Sigma), 1 mM sodium orthovanadate, 10 mM sodium pyrophosphate, 50 μM phenylarsine, 10 μM bpV, 10 mM B-glycerophosphate, 1 M sodium fluoride). Cell lysates were incubated on ice for a minimum of 30 minutes; then transferred into 1.5 ml microcentrifuge tubes and stored at −80C. Protein concentrations were determined using a BCA Assay (Pierce), according to the manufacturer's protocol.

Protein samples (20 μg) were loaded onto a Criterion XT 4-12% Bis-Tris gel (Biorad) and separated by gel electrophoresis at 120 V. Proteins were transferred to nitrocellulose membranes using the IB LOT Dry Blotting System (Invitrogen). The membrane was blocked for 1 hour at room temperature in ODYSSEY Blocking Buffer (LI-COR), followed by an overnight incubation at 4 C with primary antibodies diluted in Odyssey blocking buffer/PBST (1:1 mixture) (DPBS (Gibco)+0.1% TWEEN 20). Antibodies against the following proteins were used: CIAP1 (1:400, R&D system AF8181, DR4 (1:500, Abcam, ab8414), DRS (1:400, Abcam, ab8416) and GAPDH (1:1000 Cell Signaling Technology, #2118). The next day, membranes were washed with PBST and incubated with secondary antibodies: IRDYE 800CW Goat anti-rabbit IgG (H+L) or IRDYE 800CW Donkey anti-goat IgG (H+L) (LI-COR) for 1 hour at room temperature. Membranes were washed once more in PBST and imaged using the ODYSSEY CLx Imaging system (LI-COR).

### Cell Viability Assay

Colorectal cell lines were cultured in complete media to sub-confluency. The cells were detached with trypsin (Gibco Life Technologies, Grand Island, N.Y., USA), washed once with PBS (Gibco Life Technologies,) and suspended in complete fresh growth media. The cells were seeded in 96-well plates at optimal density as given in Tables 1A and 1B, and allowed to recover overnight at 37 ° C. Cells were then treated with increasing concentrations of in-house compound Fc-T191 (0-30 nm, 3-fold dilutions) as a single agent. At 24 hour post treatment, viability was determined by measuring the amount of cellular ATP using CellTiter-Glo Luminescent Assay (CTG, Promega Life Sciences, Madison, Wis., USA) according to the manufacturer's protocol. Luminescence values were normalized to untreated controls. Dose-response curves were generated using MATLAB (Natick, Mass., USA).

### Results

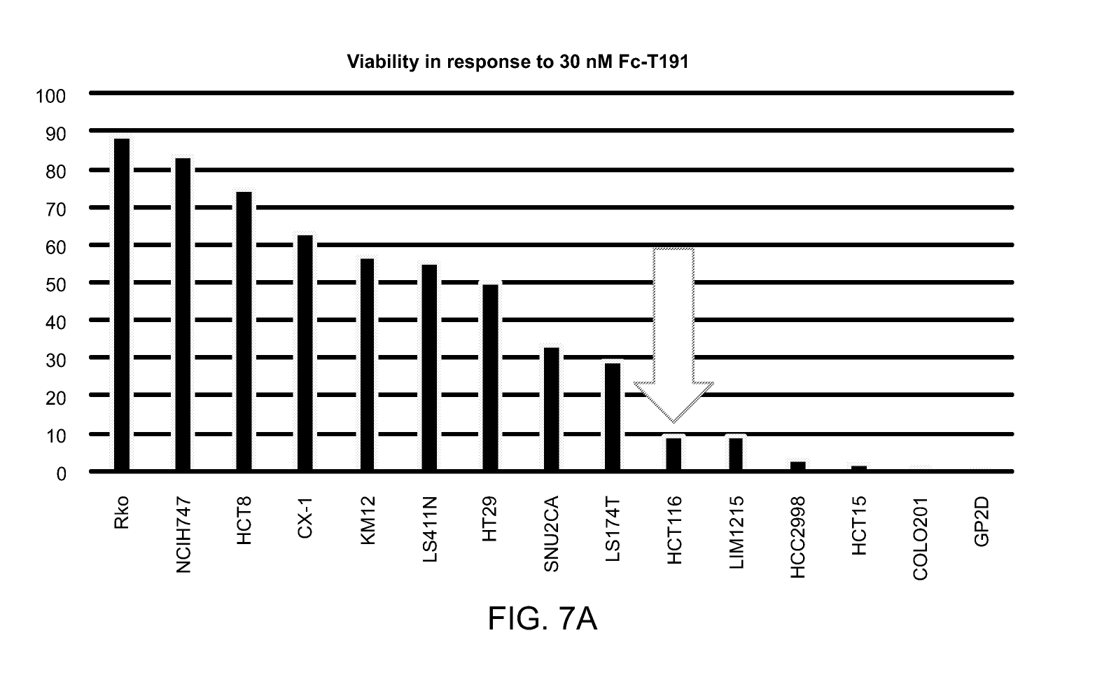

SiRNA-mediated knockdown of DR4, but not DR5, in cIAP1 (BIRC2) low LIM1215 cells made them less responsive to Fc-T191, as compared to cells transfected scrambled (non-targeting) siRNA (FIGS. 6A-6C). However, combined knockdown of DR4 and DR5, further protected the cells from Fc-T191 compared to DR4 knockdown alone (FIGS. 6B-6C).

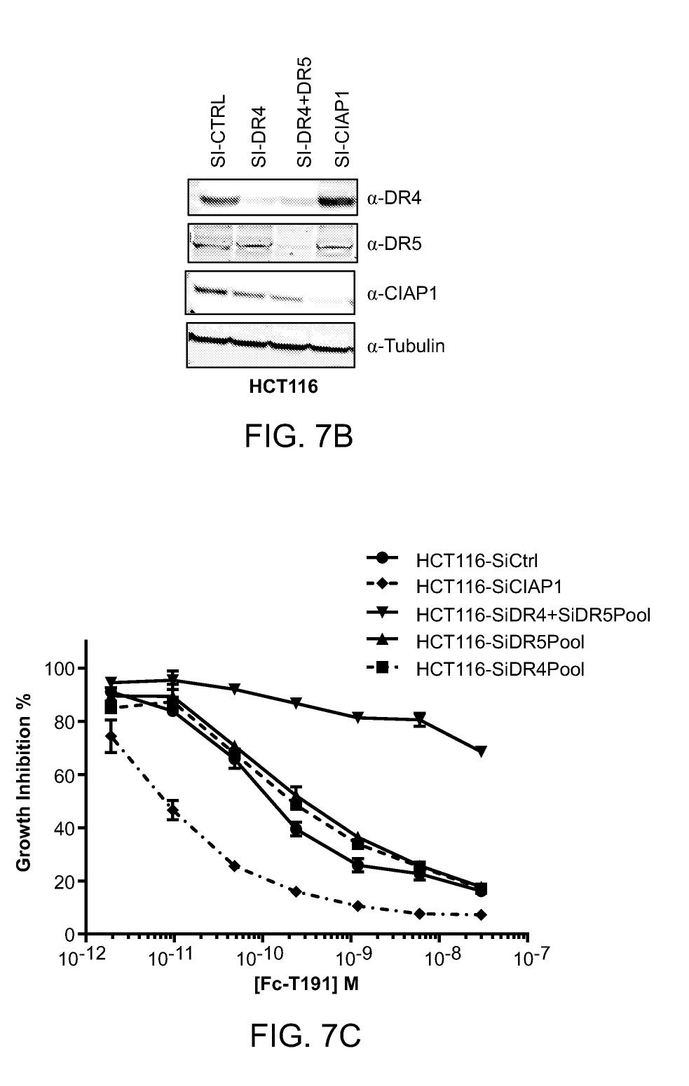

Knockdown of DR4 or DR5 alone had no significant effect on in high cIAP (BIRC2) HCT116 cells, as compared to cells transfected with scrambled siRNA (FIGS. 7A-7C).

However, combined knockdown of DR4 and DR5 rendered HCT116 cells more resistant to even high concentrations of Fc-T191 (FIGS. 7B-7C). Notably, knockdown of cIAP1 (BIRC-2) further sensitized HCT116 cells to Fc-T191 (FIGS. 7B-7C). These results suggest that cell death induced by Fc-T191 via DR4 or DRS is cell type dependent, and cIAP1 may lead to resistance to Fc-T191 induced cell death.

### Example 5: TRAIL-Sensitive PDX Models Express Relatively High DR4 (TNFRSF10A) and Low cIAP1 (BIRC2) Levels.

To further validate the biomarker hypothesis, a patient-derived xenograft (PDX) colorectal cancer model was used to assess biomarker levels and sensitivity to rhTRAIL and Fc-T191 treatment (patient samples: TX-CRC-066, -096, -088, -206, -169, and -171).

### Materials and Methods

### Cell Line-Derived Xenograft Mouse Models of Colorectal Cancer

Nude mice (NU-Foxnlnu; Charles River Laboratories) at 6 weeks old and 18-20 g body weight were injected subcutaneously in the right flank with a suspension of RKO or HCT116 cell lines (5e6) in 50% matrigel (Corning). Tumor measurements were made using a digital caliper and tumor volumes were calculated using the following equation: π/6(L×W∧2) with the “W” being the maximum width and the “L” being the maximum length. When the tumors were of sufficient size (100-250 mm³), the mice were randomized into control and experimental groups. Mice received either PBS (pH 7.4) as vehicle control or Fc-T191 at the indicated dose. Tumor volumes and body weights were monitored during the study. Following the last measurement, tumors were harvested for histological evaluation. To determine the statistical difference between the treatment groups, t-test analysis was performed using the fractional change in tumor volume at day 22 post-inoculation.

### Patient-Derived Xenograft Mouse Models of Colorectal Cancer

TX-CRC-066, -096, -088, -206, -169, -171, patient-derived colorectal cancer fragments were obtained from Texas Tech University, Cancer Center. 5- to 8-week-old female NOD-SCID mice were purchased from Charles River Laboratories (Wilmington, Mass.). Under isoflurane inhalational anesthesia, Fragments of 30 to 60 mm³ were transplanted subcutaneously into the dorsal flank of NOD-SCID mice after opening a small pocket under the skin. Incision sites were stapled with surgical staples. Mice were then monitored for tumor growth during the study. Tumor measurements were made using a digital caliper and tumor volumes were calculated using the following equation: (L×W∧2)/2 with the “W” being the maximum width and the “L” being the maximum length. Successful xenografts were passaged subsequently into new host mice. At passage 3 or 4, when the volume of the grafted tumor reached a sufficient size (˜100-250 mm³) the mice were randomized into groups for control and experimental arms, with 5 to 7 animals each. Animals were treated either with PBS or Fc-T191. Tumor volumes and body weight were monitored twice a week throughout the study. Following the last measurement, tumors were harvested for histological evaluation (IHC).

### Reverse Transcriptase and PCR Assays

Colorectal cell lines were cultured in complete media to sub-confluency. Trypsinized cells washed with PBS and total RNA was isolated using the RNeasy Kit from Qiagen as described according to the manufacturer's instructions. The same kit was used to isolate RNA from PDX tissue. 1 μg of total RNA was then reverse transcribed using the Applied Biosystems High Capacity cDNA Reverse Transcription Kit (Life Technologies, Grand Island, N.Y., USA) according to manufacturer's protocol. To quantify the transcribed gene-specific RNA, cDNA (3 technical replicates for each TaqMan Assay) was then amplified using TaqMan® Fast Advanced Master Mix and TaqMan Gene Expression Assays for TNFRSF10A, BIRC2, ACTB, RPL4 and PPP2CA (all from Applied Biosystems).

The reactions were run on a QuantStudio 12K Flex Real-Time PCR System (Life Technologies, Grand Island, N.Y., USA) under cycling condition recommended by Life Technologies. The threshold cycle (Ct) values were determinate using default threshold settings. Ct is defined as fractional cycle number at which the fluorescence passes the fixed threshold. The 2−ΔCt equation was applied to calculate the RNA expression of TNFRSF10A, BIRC2 where ΔCt=Ct (TNFRSF10A or BIRC2 gene)−Ct (geometric mean of housekeeping genes).

### Results

TX-CRC-066, -096, -088 and -206 were classified as sensitive tumor models because of the significant reduction in tumor size following Fc-T191 treatment (5 mg/kg once/week). In contrast, resistant PDX models −169 and −171 showed no significant growth inhibition in response to similar concentration and scheduling of Fc-T191. The in vivo response of TX-CRC-096 and TX-CRC-0169 to Fc-T191 is shown in FIGS. 6A and 6B as examples of a TRAIL-sensitive and a TRAIL-resistant PDX model, respectively.

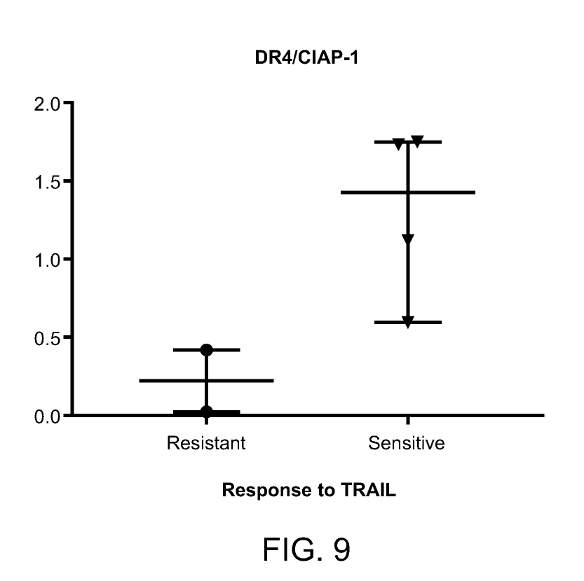

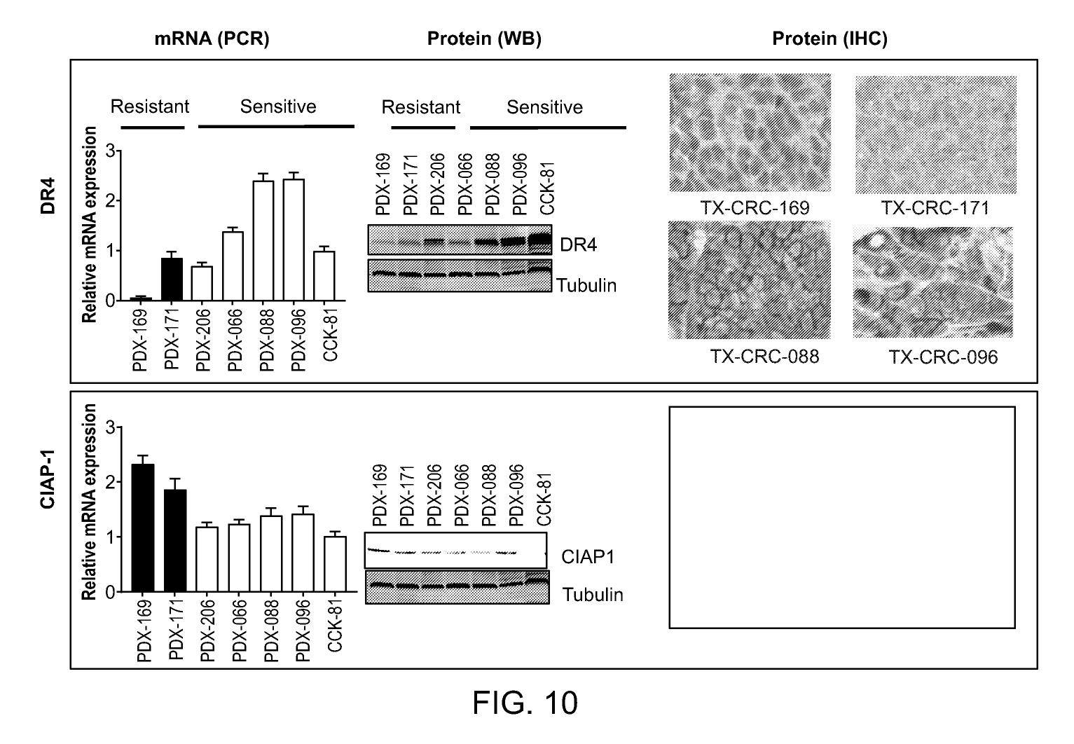

The expression of DR4 (TNFRSF10A) and cIAP1 (BIRC2) in sensitive and resistant PDX models was also tested and correlated with response to TRAIL therapy. As expected, the four sensitive PDX models showed higher DR4/cIAP1 mRNA expression ratio when compared to 2 resistant PDX models (FIG. 9). Further protein level measurement by Western blot analysis and/or IHC further confirmed the higher expression of DR4 and lower expression cIAP1 in sensitive PDX models as compared to resistant models (FIG. 10). These results further suggested that high DR4 (TNFRSF10A) and low cIAP1 (BIRC2) levels are predictive for response to TRAIL therapy.

### Equivalents

Those skilled in the art will recognize, or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

### Incorporation by Reference

The disclosure of each and every U.S. and foreign patent and pending patent application and publication referred to herein is specifically incorporated herein by reference in its entirety, as are the contents of any Sequence Listing and Figures.

SEQUENCE LISTING

| | | SEQ |
|---|---|---|
| | | ID |
| Name | Sequence | NO: |
| Human | MAMMEVQGGPSLGQTCVLIVIFTVLLQSLCVAVT | 1 |
| TRAIL | YVYFTNELKQMQDKYSKSGIACFLKEDDSYWDP | |
| | NDEESMNSPCWQVKWQLRQLVRKMILRTSEETIS | |
| | TVQEKQQNISPLVRERGPQRVAAHITGTRGRSNTL | |
| | SSPNSKNEKALGRKINSWESSRSGHSFLSNLHLRN | |
| | GELVIHEKGFYYIYSQTYFRFQEEIKENTKNDKQM | |
| | VQYIYKYTSYPDPILLMKSARNSCWSKDAEYGLY | |
| | SIYQGGIFELKENDRIFVSVTNEHLIDMDHEASFFG | |
| | AFLVG | |
| Human | MAPPPARVHLGAFLAVTPNPGSAASGTEAAAATP | 2 |
| DR4 | SKVWGSSAGRIEPRGGGRGALPTSMGQHGPSARA | |
| (TNFRS | RAGRAPGPRPAREASPRLRVHKTFKFVVVGVLLQ | |
| F10A) | VVPSSAATIKLHDQSIGTQQWEHSPLGELCPPGSH | |
| | RSEHPGACNRCTEGVGYTNASNNLFACLPCTACK | |
| | SDEEERSPCTTTRNTACQCKPGTFRNDNSAEMCR | |
| | KCSRGCPRGMVKVKDCTPWSDIECVHKESGNGH | |
| | NIWVILVVTLVVPLLLVAVLIVCCCIGSGCGGDPK | |
| | CMDRVCFWRLGLLRGPGAEDNAHNEILSNADSLS | |
| | TFVSEQQMESQEPADLTGVTVQSPGEAQCLLGPA | |
| | EAEGSQRRRLLVPANGADPTETLMLFFDKFANIVP | |
| | FDSWDQLMRQLDLTKNEIDVVRAGTAGPGDALY | |
| | AMLMKWVNKTGRNASIHTLLDALERMEERHARE | |
| | KIQDLLVDSGKFIYLEDGTGSAVSLE | |
| Human | MHKTASQRLFPGPSYQNIKSIMEDSTILSDWTNSN | 3 |
| cIAP1 | KQKMKYDFSCELYRMSTYSTFPAGVPVSERSLAR | |
| (BIRC2) | AGFYYTGVNDKVKCFCCGLMLDNWKLGDSPIQK | |
| | HKQLYPSCSFIQNLVSASLGSTSKNTSPMRNSFAH | |
| | SLSPTLEHSSLFSGSYSSLSPNPLNSRAVEDISSSR | |
| | TNPYSYAMSTEEARFLTYHMWPLTFLSPSELARAG | |
| | FYYIGPGDRVACFACGGKLSNWEPKDDAMSEHR | |
| | RHFPNCPFLENSLETLRFSISNLSMQTHAARMRTF | |
| | MYWPSSVPVQPEQLASAGFYYVGRNDDVKCFCC | |
| | DGGLRCWESGDDPWVEHAKWFPRCEFLIRMKGQ | |
| | EFVDEIQGRYPHLLEQLLSTSDTTGEENADPPIIHF | |
| | GPGESSSEDAVMMNTPVVKSALEMGFNRDLVKQ | |
| | TVQSKILTTGENYKTVNDIVSALLNAEDEKREEEK | |
| | EKQAEEMASDDLSLIRKNRMALFQQLTCVLPILD | |
| | NLLKANVINKQEHDIIKQKTQIPLQARELIDTILVK | |
| | GNAAANIFKNCLKEIDSTLYKNLFVDKNMKYIPT | |
| | EDVSGLSLEEQLRRLQEERTCKVCMDKEVSVVFIP | |
| | CGHLVVCQECAPSLRKCPICRGIIKGTVRTFLS | |
| T148 | VRERGPQIVAAHITGTRGRSNTLSSPNSKNEKALG | 4 |
| | RKINSWESSRSGHSFLSNLHLRNGELVIHEKGFYY | |
| | IYSQTYFRFQEEIKENTKNDKQMVQYIYKYTSYPD | |
| | PILLMKSARNSCWSKDAEYGLYSIYQGGVFELKE | |
| | NDRIFVSVTNEHLIDMDHEASFFGAFLVGGGGGS | |
| | GGGGSGGGGSVRERGPQIVAAHITGTRGRSNTLSS | |
| | PNSKNEKALGRKINSWESSRSGHSFLSNLHLRNGE | |
| | LVIHEKGFYYIYSQTYFRFQEEIKENTKNDKQMV | |
| | QYIYKYTSYPDPILLMKSARNSCWSKDAEYGLYSI | |
| | YQGGVFELKENDRIFVSVTNEHLIDMDHEASFFG | |
| | AFLVGGGGGSGGGGSGGGGSVRERGPQIVAAHIT | |
| | GTRGRSNTLSSPNSKNEKALGRKINSWESSRSGHS | |
| | FLSNLHLRNGELVIHEKGFYYIYSQTYFRFQEEIKE | |
| | NTKNDKQMVQYIYKYTSYPDPILLMKSARNSCW | |
| | SKDAEYGLYSIYQGGVFELKENDRIFVSVTNEHLI | |
| | DMDHEASFFGAFLVG | |
| T151 | VRERGPQRVAAHITGTRGRSNTLSSPNSKNEKAL | 5 |
| | GRKINSWESSRSGHSFLSNLHLRNGELVIHEKGFY | |
| | YIYSQTYFRFQEEIKENTKNDKQMVQYIYKYTSYP | |
| | DPILLMKSARSSCWSKDAEYGLYSIYQGGVFELK | |
| | ENDRIFVSVTNEHLIDMDHEASFFGAFLVGGGGG | |
| | SGGGGSGGGGSVRERGPQRVAAHITGTRGRSNTL | |
| | SSPNSKNEKALGRKINSWESSRSGHSFLSNLHLRN | |
| | GELVIHEKGFYYIYSQTYFRFQEEIKENTKNDKQM | |
| | VQYIYKYTSYPDPILLMKSARSSCWSKDAEYGLY | |
| | SIYQGGVFELKENDRIFVSVTNEHLIDMDHEASFF | |
| | GAFLVGGGGGSGGGGSGGGGSVRERGPQRVAAH | |
| | ITGTRGRSNTLSSPNSKNEKALGRKINSWESSRSG | |
| | HSFLSNLHLRNGELVIHEKGFYYIYSQTYFRFQEEI | |
| | KENTKNDKQMVQYIYKYTSYPDPILLMKSARSSC | |
| | WSKDAEYGLYSIYQGGVFELKENDRIFVSVTNEH | |
| | LIDMDHEASFFGAFLVG | |
| T153 | VRERGPQRVAAHITGTGGRSNTLSSPNSKNEKAL | 6 |
| | GRKINSWESSRSGHSFLSNLHLRNGELVIHEKGFY | |
| | YIYSQTYFRFQEEIKENTKNDKQMVQYIYKYTSYP | |
| | DPILLMKSARNSCWSKDAEYGLYSIYQGGVFELK | |
| | ENDRIFVSVTNEHLIDMDHEASFFGAFLVGGGGG | |
| | SGGGGSGGGGSVRERGPQRVAAHITGTGGRSNTL | |
| | SSPNSKNEKALGRKINSWESSRSGHSFLSNLHLRN | |
| | GELVIHEKGFYYIYSQTYFRFQEEIKENTKNDKQM | |
| | VQYIYKYTSYPDPILLMKSARNSCWSKDAEYGLY | |
| | SIYQGGVFELKENDRIFVSVTNEHLIDMDHEASFF | |
| | GAFLVGGGGGSGGGGSGGGGSVRERGPQRVAAH | |
| | ITGTGGRSNTLSSPNSKNEKALGRKINSWESSRSG | |
| | HSFLSNLHLRNGELVIHEKGFYYIYSQTYFRFQEEI | |
| | KENTKNDKQMVQYIYKYTSYPDPILLMKSARNSC | |
| | WSKDAEYGLYSIYQGGVFELKENDRIFVSVTNEH | |
| | LIDMDHEASFFGAFLVG | |
| T182 | VRERGPQRVAAHITGTRGRSNTLSSPNSKNEKAL | 7 |
| | GRKINSWESSRSGHSFLSNLHLRNGELVIHEKGFY | |
| | YIYSQTYFRFQEEIKENTKNDKQMVQYIYKWTDY | |
| | PDPILLMKSARNSCWSKDAEYGLYSIYQGGIFELK | |
| | ENDRIFVSVTNEHLIDMDHEASFFGAFLVGGGGG | |
| | SGGGGSGGGGSVRERGPQRVAAHITGTRGRSNTL | |
| | SSPNSKNEKALGRKINSWESSRSGHSFLSNLHLRN | |
| | GELVIHEKGFYYIYSQTYFRFQEEIKENTKNDKQM | |
| | VQYIYKWTDYPDPILLMKSARNSCWSKDAEYGL | |
| | YSIYQGGIFELKENDRIFVSVTNEHLIDMDHEASFF | |
| | GAFLVGGGGGSGGGGSGGGGSVRERGPQRVAAH | |
| | ITGTRGRSNTLSSPNSKNEKALGRKINSWESSRSG | |
| | HSFLSNLHLRNGELVIHEKGFYYIYSQTYFRFQEEI | |
| | KENTKNDKQMVQYIYKWTDYPDPILLMKSARNS | |
| | CWSKDAEYGLYSIYQGGIFELKENDRIFVSVTNEH | |
| | LIDMDHEASFFGAFLVG | |
| T183 | VRERGPQIVAAHITGTGGRSNTLSSPNSKNEKALG | 8 |
| | RKINSWESSRSGHSFLSNLHLRNGELVIHEKGFYY | |
| | IYSQTYFRFQEEIKENTKNDKQMVQYIYKWTDYP | |
| | DPILLMKSARNSCWSKDAEYGLYSIYQGGVFELK | |
| | ENDRIFVSVTNEHLIDMDHEASFFGAFLVGGGGG | |
| | SGGGGSGGGGSVRERGPQIVAAHITGTGGRSNTL | |
| | SSPNSKNEKALGRKINSWESSRSGHSFLSNLHLRN | |
| | GELVIHEKGFYYIYSQTYFRFQEEIKENTKNDKQM | |
| | VQYIYKWTDYPDPILLMKSARNSCWSKDAEYGL | |
| | YSIYQGGVFELKENDRIFVSVTNEHLIDMDHEASF | |
| | FGAFLVGGGGGSGGGGSGGGGSVRERGPQIVAA | |
| | HITGTGGRSNTLSSPNSKNEKALGRKINSWESSRS | |
| | GHSFLSNLHLRNGELVIHEKGFYYIYSQTYFRFQE | |
| | EIKENTKNDKQMVQYIYKWTDYPDPILLMKSARN | |
| | SCWSKDAEYGLYSIYQGGVFELKENDRIFVSVTN | |
| | EHLIDMDHEASFFGAFLVG | |
| T186 | VRERGPQRVAAHITGTGGRSNTLSSPNSKNEKAL | 9 |
| | GRKINSWESSRSGHSFLSNLHLRNGELVIHEKGFY | |
| | YIYSQTYFRFQEEIKENTKNDKQMVQYIYKWTDY | |
| | PDPILLMKSARNSCWSKDAEYGLYSIYQGGVFEL | |
| | KENDRIFVSVTNEHLIDMDHEASFFGAFLVGGGG | |
| | GSGGGGSGGGGSVRERGPQRVAAHITGTGGRSNT | |
| | LSSPNSKNEKALGRKINSWESSRSGHSFLSNLHLR | |
| | NGELVIHEKGFYYIYSQTYFRFQEEIKENTKNDKQ | |
| | MVQYIYKWTDYPDPILLMKSARNSCWSKDAEYG | |
| | LYSIYQGGVFELKENDRIFVSVTNEHLIDMDHEAS | |
| | FFGAFLVGGGGGSGGGGSGGGGSVRERGPQRVA | |
| | AHITGTGGRSNTLSSPNSKNEKALGRKINSWESSR | |
| | SGHSFLSNLHLRNGELVIHEKGFYYIYSQTYFRFQ | |
| | EEIKENTKNDKQMVQYIYKWTDYPDPILLMKSAR | |
| | NSCWSKDAEYGLYSIYQGGVFELKENDRIFVSVT | |
| | NEHLIDMDHEASFFGAFLVG | |
| T191 | VRERGPQRVAAHITGTGGRSNTLSSPNSKNEKAL | 10 |
| | GRKINSWESSRSGHSFLSNLHLRNGELVIHEKGFY | |
| | YIYSQTYFRFQEEIKENTKNDKQMVQYIYKWTDY | |
| | PDPILLMKSARSSCWSKDAEYGLYSIYQGGVFEL | |
| | KENDRIFVSVTNEHLIDMDHEASFFGAFLVGGGG | |
| | GSGGGGSGGGGSVRERGPQRVAAHITGTGGRSNT | |
| | LSSPNSKNEKALGRKINSWESSRSGHSFLSNLHLR | |
| | NGELVIHEKGFYYIYSQTYFRFQEEIKENTKNDKQ | |
| | MVQYIYKWTDYPDPILLMKSARSSCWSKDAEYG | |
| | LYSIYQGGVFELKENDRIFVSVTNEHLIDMDHEAS | |
| | FFGAFLVGGGGGSGGGGSGGGGSVRERGPQRVA | |
| | AHITGTGGRSNTLSSPNSKNEKALGRKINSWESSR | |
| | SGHSFLSNLHLRNGELVIHEKGFYYIYSQTYFRFQ | |
| | EEIKENTKNDKQMVQYIYKWTDYPDPILLMKSAR | |
| | SSCWSKDAEYGLYSIYQGGVFELKENDRIFVSVT | |
| | NEHLIDMDHEASFFGAFLVG | |
| T196 | VRERGPQIVAAHITGTGGRSNTLSSPNSKNEKALG | 11 |
| | RKINSWESSRSGHSFLSNLHLRNGELVIHEKGFYY | |
| | IYSQTYFRFQEEIKENTKNDKQMVQYIYKYTSYPD | |
| | PILLMKSARNSCWSKDAEYGLYSIYQGGVFELKE | |
| | NDRIFVSVTNEHLIDMDHEASFFGAFLVGGGGGS | |
| | GGGGSGGGGSVRERGPQIVAAHITGTGGRSNTLS | |
| | SPNSKNEKALGRKINSWESSRSGHSFLSNLHLRNG | |
| | ELVIHEKGFYYIYSQTYFRFQEEIKENTKNDKQMV | |
| | QYIYKYTSYPDPILLMKSARNSCWSKDAEYGLYSI | |
| | YQGGVFELKENDRIFVSVTNEHLIDMDHEASFFG | |
| | AFLVGGGGGSGGGGSGGGGSVRERGPQIVAAHIT | |
| | GTGGRSNTLSSPNSKNEKALGRKINSWESSRSGHS | |
| | FLSNLHLRNGELVIHEKGFYYIYSQTYFRFQEEIKE | |
| | NTKNDKQMVQYIYKYTSYPDPILLMKSARNSCW | |
| | SKDAEYGLYSIYQGGVFELKENDRIFVSVTNEHLI | |
| | DMDHEASFFGAFLVG | |
| T202 | VRERGPQRVAAHITGTGGRSNTLSSPNSKNEKAL | 12 |
| | GRKINSWESSRSGHSFLSNLHLRNGELVIHEKGFY | |
| | YIYSQTYFRFQEEIKENTKNDKQMVQYIYKYTSYP | |
| | DPILLMKSARSSCWSKDAEYGLYSIYQGGVFELK | |
| | ENDRIFVSVTNEHLIDMDHEASFFGAFLVGGGGG | |
| | SGGGGSGGGGSVRERGPQRVAAHITGTGGRSNTL | |
| | SSPNSKNEKALGRKINSWESSRSGHSFLSNLHLRN | |
| | GELVIHEKGFYYIYSQTYFRFQEEIKENTKNDKQM | |
| | VQYIYKYTSYPDPILLMKSARSSCWSKDAEYGLY | |
| | SIYQGGVFELKENDRIFVSVTNEHLIDMDHEASFF | |
| | GAFLVGGGGGSGGGGSGGGGSVRERGPQRVAAH | |
| | ITGTGGRSNTLSSPNSKNEKALGRKINSWESSRSG | |
| | HSFLSNLHLRNGELVIHEKGFYYIYSQTYFRFQEEI | |
| | KENTKNDKQMVQYIYKYTSYPDPILLMKSARSSC | |
| | WSKDAEYGLYSIYQGGVFELKENDRIFVSVTNEH | |
| | LIDMDHEASFFGAFLVG | |
| T203 | VRERGPQIVAAHITGTGGRSNTLSSPNSKNEKALG | 13 |
| | RKINSWESSRSGHSFLSNLHLRNGELVIHEKGFYY | |
| | IYSQTYFRFQEEIKENTKNDKQMVQYIYKYTSYPD | |
| | PILLMKSARSSCWSKDAEYGLYSIYQGGVFELKE | |
| | NDRIFVSVTNEHLIDMDHEASFFGAFLVGGGGGS | |
| | GGGGSGGGGSVRERGPQIVAAHITGTGGRSNTLS | |
| | SPNSKNEKALGRKINSWESSRSGHSFLSNLHLRNG | |
| | ELVIHEKGFYYIYSQTYFRFQEEIKENTKNDKQMV | |
| | QYIYKYTSYPDPILLMKSARSSCWSKDAEYGLYSI | |
| | YQGGVFELKENDRIFVSVTNEHLIDMDHEASFFG | |
| | AFLVGGGGGSGGGGSGGGGSVRERGPQIVAAHIT | |
| | GTGGRSNTLSSPNSKNEKALGRKINSWESSRSGHS | |
| | FLSNLHLRNGELVIHEKGFYYIYSQTYFRFQEEIKE | |
| | NTKNDKQMVQYIYKYTSYPDPILLMKSARSSCWS | |
| | KDAEYGLYSIYQGGVFELKENDRIFVSVTNEHLID | |
| | MDHEASFFGAFLVG | |
| T204 | VRERGPQIVAAHITGTRGRSNTLSSPNSKNEKALG | 14 |
| | RKINSWESSRSGHSFLSNLHLRNGELVIHEKGFYY | |
| | IYSQTYFRFQEEIKENTKNDKQMVQYIYKYTSYPD | |
| | PILLMKSARNSCWSKDAEYGLYSIYQGGVFELKE | |
| | NDRIFVSVTNEHLIDMDHEASFFGAFLVGGGGGS | |
| | GGGGSGGGGSVRERGPQIVAAHITGTRGRSNTLSS | |
| | PNSKNEKALGRKINSWESSRSGHSFLSNLHLRNGE | |
| | LVIHEKGFYYIYSQTYFRFQEEIKENTKNDKQMV | |
| | QYIYKYTSYPDPILLMKSARNSCWSKDAEYGLYSI | |
| | YQGGVFELKENDRIFVSVTNEHLIDMDHEASFFG | |
| | AFLVGGGGGSGGGGSGGGGSVRERGPQIVAAHIT | |
| | GTRGRSNTLSSPNSKNEKALGRKINSWESSRSGHS | |
| | FLSNLHLRNGELVIHEKGFYYIYSQTYFRFQEEIKE | |
| | NTKNDKQMVQYIYKYTSYPDPILLMKSARNSCW | |
| | SKDAEYGLYSIYQGGVFELKENDRIFVSVTNEHLI | |
| | DMDHEASFFGAFLVG | |
| T205 | VRERGPQRVAAHITGTGGRSNTLSSPNSKNEKAL | 15 |
| | GRKINSWESSRSGHSFLSNLHLRNGELVIHEKGFY | |
| | YIYSQTYFRFQEEIKENTKNDKQMVQYIYKYTSYP | |
| | DPILLMKSARNSCWSKDAEYGLYSIYQGGVFELK | |
| | ENDRIFVSVTNEHLIDMDHEASFFGAFLVGGGGG | |
| | SGGGGSGGGGSVRERGPQRVAAHITGTGGRSNTL | |
| | SSPNSKNEKALGRKINSWESSRSGHSFLSNLHLRN | |
| | GELVIHEKGFYYIYSQTYFRFQEEIKENTKNDKQM | |
| | VQYIYKYTSYPDPILLMKSARNSCWSKDAEYGLY | |
| | SIYQGGVFELKENDRIFVSVTNEHLIDMDHEASFF | |
| | GAFLVGGGGGSGGGGSGGGGSVRERGPQRVAAH | |
| | ITGTGGRSNTLSSPNSKNEKALGRKINSWESSRSG | |
| | HSFLSNLHLRNGELVIHEKGFYYIYSQTYFRFQEEI | |
| | KENTKNDKQMVQYIYKYTSYPDPILLMKSARNSC | |
| | WSKDAEYGLYSIYQGGVFELKENDRIFVSVTNEH | |
| | LIDMDHEASFFGAFLVG | |
| T206 | VRERGPQRVAAHITGTRGRSNTLSSPNSKNEKAL | 16 |
| | GRKINSWESSRSGHSFLSNLHLRNGELVIHEKGFY | |
| | YIYSQTYFRFQEEIKENTKNDKQMVQYIYKYTSYP | |
| | DPILLMKSARSSCWSKDAEYGLYSIYQGGVFELK | |
| | ENDRIFVSVTNEHLIDMDHEASFFGAFLVGGGGG | |
| | SGGGGSGGGGSVRERGPQRVAAHITGTRGRSNTL | |
| | SSPNSKNEKALGRKINSWESSRSGHSFLSNLHLRN | |
| | GELVIHEKGFYYIYSQTYFRFQEEIKENTKNDKQM | |
| | VQYIYKYTSYPDPILLMKSARSSCWSKDAEYGLY | |
| | SIYQGGVFELKENDRIFVSVTNEHLIDMDHEASFF | |
| | GAFLVGGGGGSGGGGSGGGGSVRERGPQRVAAH | |
| | ITGTRGRSNTLSSPNSKNEKALGRKINSWESSRSG | |
| | HSFLSNLHLRNGELVIHEKGFYYIYSQTYFRFQEEI | |
| | KENTKNDKQMVQYIYKYTSYPDPILLMKSARSSC | |
| | WSKDAEYGLYSIYQGGVFELKENDRIFVSVTNEH | |
| | LIDMDHEASFFGAFLVG | |
| T207 | VRERGPQIVAAHITGTRGRSNTLSSPNSKNEKALG | 17 |
| | RKINSWESSRSGHSFLSNLHLRNGELVIHEKGFYY | |
| | IYSQTYFRFQEEIKENTKNDKQMVQYIYKYTSYPD | |
| | PILLMKSARSSCWSKDAEYGLYSIYQGGVFELKE | |
| | NDRIFVSVTNEHLIDMDHEASFFGAFLVGGGGGS | |
| | GGGGSGGGGSVRERGPQIVAAHITGTRGRSNTLSS | |
| | PNSKNEKALGRKINSWESSRSGHSFLSNLHLRNGE | |
| | LVIHEKGFYYIYSQTYFRFQEEIKENTKNDKQMV | |
| | QYIYKYTSYPDPILLMKSARSSCWSKDAEYGLYSI | |
| | YQGGVFELKENDRIFVSVTNEHLIDMDHEASFFG | |
| | AFLVGGGGGSGGGGSGGGGSVRERGPQIVAAHIT | |
| | GTRGRSNTLSSPNSKNEKALGRKINSWESSRSGHS | |
| | FLSNLHLRNGELVIHEKGFYYIYSQTYFRFQEEIKE | |
| | NTKNDKQMVQYIYKYTSYPDPILLMKSARSSCWS | |
| | KDAEYGLYSIYQGGVFELKENDRIFVSVTNEHLID | |
| | MDHEASFFGAFLVG | |
| T208 | VRERGPQIVAAHITGTGGRSNTLSSPNSKNEKALG | 18 |
| | RKINSWESSRSGHSFLSNLHLRNGELVIHEKGFYY | |
| | IYSQTYFRFQEEIKENTKNDKQMVQYIYKYTSYPD | |
| | PILLMKSARNSCWSKDAEYGLYSIYQGGIFELKEN | |
| | DRIFVSVTNEHLIDMDHEASFFGAFLVGGGGGSG | |
| | GGGSGGGGSVRERGPQIVAAHITGTGGRSNTLSSP | |
| | NSKNEKALGRKINSWESSRSGHSFLSNLHLRNGEL | |
| | VIHEKGFYYIYSQTYFRFQEEIKENTKNDKQMVQ | |
| | YIYKYTSYPDPILLMKSARNSCWSKDAEYGLYSIY | |
| | QGGIFELKENDRIFVSVTNEHLIDMDHEASFFGAF | |
| | LVGGGGGSGGGGSGGGGSVRERGPQIVAAHITGT | |
| | GGRSNTLSSPNSKNEKALGRKINSWESSRSGHSFL | |
| | SNLHLRNGELVIHEKGFYYIYSQTYFRFQEEIKEN | |
| | TKNDKQMVQYIYKYTSYPDPILLMKSARNSCWS | |
| | KDAEYGLYSIYQGGIFELKENDRIFVSVTNEHLID | |
| | MDHEASFFGAFLVG | |
| T209 | VRERGPQIVAAHITGTGGRSNTLSSPNSKNEKALG | 19 |
| | RKINSWESSRSGHSFLSNLHLRNGELVIHEKGFYY | |
| | IYSQTYFRFQEEIKENTKNDKQMVQYIYKYTSYPD | |
| | PILLMKSARSSCWSKDAEYGLYSIYQGGIFELKEN | |
| | DRIFVSVTNEHLIDMDHEASFFGAFLVGGGGGSG | |
| | GGGSGGGGSVRERGPQIVAAHITGTGGRSNTLSSP | |
| | NSKNEKALGRKINSWESSRSGHSFLSNLHLRNGEL | |
| | VIHEKGFYYIYSQTYFRFQEEIKENTKNDKQMVQ | |
| | YIYKYTSYPDPILLMKSARSSCWSKDAEYGLYSIY | |
| | QGGIFELKENDRIFVSVTNEHLIDMDHEASFFGAF | |
| | LVGGGGGSGGGGSGGGGSVRERGPQIVAAHITGT | |
| | GGRSNTLSSPNSKNEKALGRKINSWESSRSGHSFL | |
| | SNLHLRNGELVIHEKGFYYIYSQTYFRFQEEIKEN | |
| | TKNDKQMVQYIYKYTSYPDPILLMKSARSSCWSK | |
| | DAEYGLYSIYQGGIFELKENDRIFVSVTNEHLIDM | |
| | DHEASFFGAFLVG | |
| T210 | VRERGPQIVAAHITGTRGRSNTLSSPNSKNEKALG | 20 |
| | RKINSWESSRSGHSFLSNLHLRNGELVIHEKGFYY | |
| | IYSQTYFRFQEEIKENTKNDKQMVQYIYKYTSYPD | |
| | PILLMKSARSSCWSKDAEYGLYSIYQGGIFELKEN | |
| | DRIFVSVTNEHLIDMDHEASFFGAFLVGGGGGSG | |
| | GGGSGGGGSVRERGPQIVAAHITGTRGRSNTLSSP | |
| | NSKNEKALGRKINSWESSRSGHSFLSNLHLRNGEL | |
| | VIHEKGFYYIYSQTYFRFQEEIKENTKNDKQMVQ | |
| | YIYKYTSYPDPILLMKSARSSCWSKDAEYGLYSIY | |
| | QGGIFELKENDRIFVSVTNEHLIDMDHEASFFGAF | |
| | LVGGGGGSGGGGSGGGGSVRERGPQIVAAHITGT | |
| | RGRSNTLSSPNSKNEKALGRKINSWESSRSGHSFL | |
| | SNLHLRNGELVIHEKGFYYIYSQTYFRFQEEIKEN | |
| | TKNDKQMVQYIYKYTSYPDPILLMKSARSSCWSK | |
| | DAEYGLYSIYQGGIFELKENDRIFVSVTNEHLIDM | |
| | DHEASFFGAFLVG | |
| T211 | VRERGPQRVAAHITGTGGRSNTLSSPNSKNEKAL | 21 |
| | GRKINSWESSRSGHSFLSNLHLRNGELVIHEKGFY | |
| | YIYSQTYFRFQEEIKENTKNDKQMVQYIYKYTSYP | |
| | DPILLMKSARSSCWSKDAEYGLYSIYQGGIFELKE | |
| | NDRIFVSVTNEHLIDMDHEASFFGAFLVGGGGGS | |
| | GGGGSGGGGSVRERGPQRVAAHITGTGGRSNTLS | |
| | SPNSKNEKALGRKINSWESSRSGHSFLSNLHLRNG | |
| | ELVIHEKGFYYIYSQTYFRFQEEIKENTKNDKQMV | |
| | QYIYKYTSYPDPILLMKSARSSCWSKDAEYGLYSI | |
| | YQGGIFELKENDRIFVSVTNEHLIDMDHEASFFGA | |
| | FLVGGGGGSGGGGSGGGGSVRERGPQRVAAHIT | |
| | GTGGRSNTLSSPNSKNEKALGRKINSWESSRSGHS | |
| | FLSNLHLRNGELVIHEKGFYYIYSQTYFRFQEEIKE | |
| | NTKNDKQMVQYIYKYTSYPDPILLMKSARSSCWS | |
| | KDAEYGLYSIYQGGIFELKENDRIFVSVTNEHLID | |
| | MDHEASFFGAFLVG | |
| Fc-T148 | DKTHTCPPCPAPELLGGPSVFLFPPKPKDTLMISRT | 22 |
| | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTRGRSNTLSSPNSKNEK | |
| | ALGRKINSWESSRSGHSFLSNLHLRNGELVIHEKG | |
| | FYYIYSQTYFRFQEEIKENTKNDKQMVQYIYKYTS | |
| | YPDPILLMKSARNSCWSKDAEYGLYSIYQGGVFE | |
| | LKENDRIFVSVTNEHLIDMDHEASFFGAFLVGGG | |
| | GGSGGGGSGGGGSVRERGPQIVAAHITGTRGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKYTSYPDPILLMKSARNSCWSKDAEY | |
| | GLYSIYQGGVFELKENDRIFVSVTNEHLIDMDHEA | |
| | SFFGAFLVGGGGGSGGGGSGGGGSVRERGPQIVA | |
| | AHITGTRGRSNTLSSPNSKNEKALGRKINSWESSR | |
| | SGHSFLSNLHLRNGELVIHEKGFYYIYSQTYFRFQ | |
| | EEIKENTKNDKQMVQYIYKYTSYPDPILLMKSAR | |
| | NSCWSKDAEYGLYSIYQGGVFELKENDRIFVSVT | |
| | NEHLIDMDHEASFFGAFLVG | |
| Fc-T151 | DKTHTCPPCPAPELLGGPSVFLFPPKPKDTLMISR | 23 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTRGRSNTLSSPNSKNE | |
| | KALGRKINSWESSRSGHSFLSNLHLRNGELVIHEK | |
| | GFYYIYSQTYFRFQEEIKENTKNDKQMVQYIYKY | |
| | TSYPDPILLMKSARSSCWSKDAEYGLYSIYQGGVF | |
| | ELKENDRIFVSVTNEHLIDMDHEASFFGAFLVGGG | |
| | GGSGGGGSGGGGSVRERGPQRVAAHITGTRGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKYTSYPDPILLMKSARSSCWSKDAEYG | |
| | LYSIYQGGVFELKENDRIFVSVTNEHLIDMDHEAS | |
| | FFGAFLVGGGGGSGGGGSGGGGSVRERGPQRVA | |
| | AHITGTRGRSNTLSSPNSKNEKALGRKINSWESSR | |
| | SGHSFLSNLHLRNGELVIHEKGFYYIYSQTYFRFQ | |
| | EEIKENTKNDKQMVQYIYKYTSYPDPILLMKSAR | |
| | SSCWSKDAEYGLYSIYQGGVFELKENDRIFVSVT | |
| | NEHLIDMDHEASFFGAFLVG | |
| Fc-T153 | DKTHTCPPCPAPELLGGPSVFLFPPKPKDTLMISR | 24 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTGGRSNTLSSPNSKNE | |
| | KALGRKINSWESSRSGHSFLSNLHLRNGELVIHEK | |
| | GFYYIYSQTYFRFQEEIKENTKNDKQMVQYIYKY | |
| | TSYPDPILLMKSARNSCWSKDAEYGLYSIYQGGV | |
| | FELKENDRIFVSVTNEHLIDMDHEASFFGAFLVGG | |
| | GGGSGGGGSGGGGSVRERGPQRVAAHITGTGGR | |
| | SNTLSSPNSKNEKALGRKINSWESSRSGHSFLSNL | |
| | HLRNGELVIHEKGFYYIYSQTYFRFQEEIKENTKN | |
| | DKQMVQYIYKYTSYPDPILLMKSARNSCWSKDA | |
| | EYGLYSIYQGGVFELKENDRIFVSVTNEHLIDMDH | |
| | EASFFGAFLVGGGGGSGGGGSGGGGSVRERGPQR | |
| | VAAHITGTGGRSNTLSSPNSKNEKALGRKINSWES | |
| | SRSGHSFLSNLHLRNGELVIHEKGFYYIYSQTYFR | |
| | FQEEIKENTKNDKQMVQYIYKYTSYPDPILLMKS | |
| | ARNSCWSKDAEYGLYSIYQGGVFELKENDRIFVS | |
| | VTNEHLIDMDHEASFFGAFLVG | |
| Fc-T182 | DKTHTCPPCPAPELLGGPSVFLFPPKPKDTLMISR | 25 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTRGRSNTLSSPNSKNE | |
| | KALGRKINSWESSRSGHSFLSNLHLRNGELVIHEK | |
| | GFYYIYSQTYFRFQEEIKENTKNDKQMVQYIYKW | |
| | TDYPDPILLMKSARNSCWSKDAEYGLYSIYQGGIF | |
| | ELKENDRIFVSVTNEHLIDMDHEASFFGAFLVGGG | |
| | GGSGGGGSGGGGSVRERGPQRVAAHITGTRGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKWTDYPDPILLMKSARNSCWSKDAEY | |
| | GLYSIYQGGIFELKENDRIFVSVTNEHLIDMDHEA | |
| | SFFGAFLVGGGGGSGGGGSGGGGSVRERGPQRV | |
| | AAHITGTRGRSNTLSSPNSKNEKALGRKINSWESS | |
| | RSGHSFLSNLHLRNGELVIHEKGFYYIYSQTYFRF | |
| | QEEIKENTKNDKQMVQYIYKWTDYPDPILLMKSA | |
| | RNSCWSKDAEYGLYSIYQGGIFELKENDRIFVSVT | |
| | NEHLIDMDHEASFFGAFLVG | |
| Fc-T183 | DKTHTCPPCPAPELLGGPSVFLFPPKPKDTLMISR | 26 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTGGRSNTLSSPNSKNEK | |
| | ALGRKINSWESSRSGHSFLSNLHLRNGELVIHEKG | |
| | FYYIYSQTYFRFQEEIKENTKNDKQMVQYIYKWT | |
| | DYPDPILLMKSARNSCWSKDAEYGLYSIYQGGVF | |
| | ELKENDRIFVSVTNEHLIDMDHEASFFGAFLVGGG | |
| | GGSGGGGSGGGGSVRERGPQIVAAHITGTGGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKWTDYPDPILLMKSARNSCWSKDAEY | |
| | GLYSIYQGGVFELKENDRIFVSVTNEHLIDMDHEA | |
| | SFFGAFLVGGGGGSGGGGSGGGGSVRERGPQIVA | |
| | AHITGTGGRSNTLSSPNSKNEKALGRKINSWESSR | |
| | SGHSFLSNLHLRNGELVIHEKGFYYIYSQTYFRFQ | |
| | EEIKENTKNDKQMVQYIYKWTDYPDPILLMKSAR | |
| | NSCWSKDAEYGLYSIYQGGVFELKENDRIFVSVT | |
| | NEHLIDMDHEASFFGAFLVG | |
| Fc-T186 | DKTHTCPPCPAPELLGGPSVFLFPPKPKDTLMISR | 27 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTGGRSNTLSSPNSKNE | |
| | KALGRKINSWESSRSGHSFLSNLHLRNGELVIHEK | |
| | GFYYIYSQTYFRFQEEIKENTKNDKQMVQYIYKW | |
| | TDYPDPILLMKSARNSCWSKDAEYGLYSIYQGGV | |
| | FELKENDRIFVSVTNEHLIDMDHEASFFGAFLVGG | |
| | GGGSGGGGSGGGGSVRERGPQRVAAHITGTGGR | |
| | SNTLSSPNSKNEKALGRKINSWESSRSGHSFLSNL | |
| | HLRNGELVIHEKGFYYIYSQTYFRFQEEIKENTKN | |
| | DKQMVQYIYKWTDYPDPILLMKSARNSCWSKDA | |
| | EYGLYSIYQGGVFELKENDRIFVSVTNEHLIDMDH | |
| | EASFFGAFLVGGGGGSGGGGSGGGGSVRERGPQR | |
| | VAAHITGTGGRSNTLSSPNSKNEKALGRKINSWES | |
| | SRSGHSFLSNLHLRNGELVIHEKGFYYIYSQTYFR | |
| | FQEEIKENTKNDKQMVQYIYKWTDYPDPILLMKS | |
| | ARNSCWSKDAEYGLYSIYQGGVFELKENDRIFVS | |
| | VTNEHLIDMDHEASFFGAFLVG | |
| Fc-T191 | DKTHTCPPCPAPELLGGPSVFLFPPKPKDTLMISR | 28 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTGGRSNTLSSPNSKNE | |
| | KALGRKINSWESSRSGHSFLSNLHLRNGELVIHEK | |
| | GFYYIYSQTYFRFQEEIKENTKNDKQMVQYIYKW | |
| | TDYPDPILLMKSARSSCWSKDAEYGLYSIYQGGV | |
| | FELKENDRIFVSVTNEHLIDMDHEASFFGAFLVGG | |
| | GGGSGGGGSGGGGSVRERGPQRVAAHITGTGGR | |
| | SNTLSSPNSKNEKALGRKINSWESSRSGHSFLSNL | |
| | HLRNGELVIHEKGFYYIYSQTYFRFQEEIKENTKN | |
| | DKQMVQYIYKWTDYPDPILLMKSARSSCWSKDA | |
| | EYGLYSIYQGGVFELKENDRIFVSVTNEHLIDMDH | |
| | EASFFGAFLVGGGGGSGGGGSGGGGSVRERGPQR | |
| | VAAHITGTGGRSNTLSSPNSKNEKALGRKINSWES | |
| | SRSGHSFLSNLHLRNGELVIHEKGFYYIYSQTYFR | |
| | FQEEIKENTKNDKQMVQYIYKWTDYPDPILLMKS | |
| | ARSSCWSKDAEYGLYSIYQGGVFELKENDRIFVS | |
| | VTNEHLIDMDHEASFFGAFLVG | |
| Fc-T196 | DKTHTCPPCPAPELLGGPSVFLFPPKPKDTLMISR | 29 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTGGRSNTLSSPNSKNEK | |
| | ALGRKINSWESSRSGHSFLSNLHLRNGELVIHEKG | |
| | FYYIYSQTYFRFQEEIKENTKNDKQMVQYIYKYTS | |
| | YPDPILLMKSARNSCWSKDAEYGLYSIYQGGVFE | |
| | LKENDRIFVSVTNEHLIDMDHEASFFGAFLVGGG | |
| | GGSGGGGSGGGGSVRERGPQIVAAHITGTGGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKYTSYPDPILLMKSARNSCWSKDAEY | |
| | GLYSIYQGGVFELKENDRIFVSVTNEHLIDMDHEA | |
| | SFFGAFLVGGGGGSGGGGSGGGGSVRERGPQIVA | |
| | AHITGTGGRSNTLSSPNSKNEKALGRKINSWESSR | |
| | SGHSFLSNLHLRNGELVIHEKGFYYIYSQTYFRFQ | |
| | EEIKENTKNDKQMVQYIYKYTSYPDPILLMKSAR | |
| | NSCWSKDAEYGLYSIYQGGVFELKENDRIFVSVT | |
| | NEHLIDMDHEASFFGAFLVG | |
| Fc-T202 | DKTHTCPPCPAPELLGGPSVFLFPPKPKDTLMISR | 30 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTGGRSNTLSSPNSKNE | |
| | KALGRKINSWESSRSGHSFLSNLHLRNGELVIHEK | |
| | GFYYIYSQTYFRFQEEIKENTKNDKQMVQYIYKY | |
| | TSYPDPILLMKSARSSCWSKDAEYGLYSIYQGGVF | |
| | ELKENDRIFVSVTNEHLIDMDHEASFFGAFLVGGG | |
| | GGSGGGGSGGGGSVRERGPQRVAAHITGTGGRS | |
| | NTLSSPNSKNEKALGRKINSWESSRSGHSFLSNLH | |
| | LRNGELVIHEKGFYYIYSQTYFRFQEEIKENTKND | |
| | KQMVQYIYKYTSYPDPILLMKSARSSCWSKDAEY | |
| | GLYSIYQGGVFELKENDRIFVSVTNEHLIDMDHEA | |
| | SFFGAFLVGGGGGSGGGGSGGGGSVRERGPQRV | |
| | AAHITGTGGRSNTLSSPNSKNEKALGRKINSWESS | |
| | RSGHSFLSNLHLRNGELVIHEKGFYYIYSQTYFRF | |
| | QEEIKENTKNDKQMVQYIYKYTSYPDPILLMKSA | |
| | RSSCWSKDAEYGLYSIYQGGVFELKENDRIFVSVT | |
| | NEHLIDMDHEASFFGAFLVG | |
| Fc-T203 | DKTHTCPPCPAPELLGGPSVFLFPPKPKDTLMISR | 31 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTGGRSNTLSSPNSKNEK | |
| | ALGRKINSWESSRSGHSFLSNLHLRNGELVIHEKG | |
| | FYYIYSQTYFRFQEEIKENTKNDKQMVQYIYKYTS | |
| | YPDPILLMKSARSSCWSKDAEYGLYSIYQGGVFE | |
| | LKENDRIFVSVTNEHLIDMDHEASFFGAFLVGGG | |
| | GGSGGGGSGGGGSVRERGPQIVAAHITGTGGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKYTSYPDPILLMKSARSSCWSKDAEYG | |
| | LYSIYQGGVFELKENDRIFVSVTNEHLIDMDHEAS | |
| | FFGAFLVGGGGGSGGGGSGGGGSVRERGPQIVAA | |
| | HITGTGGRSNTLSSPNSKNEKALGRKINSWESSRS | |
| | GHSFLSNLHLRNGELVIHEKGFYYIYSQTYFRFQE | |
| | EIKENTKNDKQMVQYIYKYTSYPDPILLMKSARSS | |
| | CWSKDAEYGLYSIYQGGVFELKENDRIFVSVTNE | |
| | HLIDMDHEASFFGAFLVG | |
| Fc-T204 | DKTHTCPPCPAPELLGGPSVFLFPPKPKDTLMISR | 32 |
| | PEVTCVVVDVSHEDPEVKFNWYVDGVEVHNAKT | |
| | TKPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTRGRSNTLSSPNSKNEK | |
| | ALGRKINSWESSRSGHSFLSNLHLRNGELVIHEKG | |
| | FYYIYSQTYFRFQEEIKENTKNDKQMVQYIYKYTS | |
| | YPDPILLMKSARNSCWSKDAEYGLYSIYQGGVFE | |
| | LKENDRIFVSVTNEHLIDMDHEASFFGAFLVGGG | |
| | GGSGGGGSGGGGSVRERGPQIVAAHITGTRGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKYTSYPDPILLMKSARNSCWSKDAEY | |
| | GLYSIYQGGVFELKENDRIFVSVTNEHLIDMDHEA | |
| | SFFGAFLVGGGGGSGGGGSGGGGSVRERGPQIVA | |
| | AHITGTRGRSNTLSSPNSKNEKALGRKINSWESSR | |
| | SGHSFLSNLHLRNGELVIHEKGFYYIYSQTYFRFQ | |
| | EEIKENTKNDKQMVQYIYKYTSYPDPILLMKSAR | |
| | NSCWSKDAEYGLYSIYQGGVFELKENDRIFVSVT | |
| | NEHLIDMDHEASFFGAFLVG | |
| Fc-T205 | DKTHTCPPCPAPELLGGPSVFLFPPKPKDTLMISR | 33 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTGGRSNTLSSPNSKNE | |
| | KALGRKINSWESSRSGHSFLSNLHLRNGELVIHEK | |
| | GFYYIYSQTYFRFQEEIKENTKNDKQMVQYIYKY | |
| | TSYPDPILLMKSARNSCWSKDAEYGLYSIYQGGV | |
| | FELKENDRIFVSVTNEHLIDMDHEASFFGAFLVGG | |
| | GGGSGGGGSGGGGSVRERGPQRVAAHITGTGGR | |
| | SNTLSSPNSKNEKALGRKINSWESSRSGHSFLSNL | |
| | HLRNGELVIHEKGFYYIYSQTYFRFQEEIKENTKN | |
| | DKQMVQYIYKYTSYPDPILLMKSARNSCWSKDA | |
| | EYGLYSIYQGGVFELKENDRIFVSVTNEHLIDMDH | |
| | EASFFGAFLVGGGGGSGGGGSGGGGSVRERGPQR | |
| | VAAHITGTGGRSNTLSSPNSKNEKALGRKINSWES | |
| | SRSGHSFLSNLHLRNGELVIHEKGFYYIYSQTYFR | |
| | FQEEIKENTKNDKQMVQYIYKYTSYPDPILLMKS | |
| | ARNSCWSKDAEYGLYSIYQGGVFELKENDRIFVS | |
| | VTNEHLIDMDHEASFFGAFLVG | |
| Fc-T206 | DKTHTCPPCPAPELLGGPSVFLFPPKPKDTLMISR | 34 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTRGRSNTLSSPNSKNE | |
| | KALGRKINSWESSRSGHSFLSNLHLRNGELVIHEK | |
| | GFYYIYSQTYFRFQEEIKENTKNDKQMVQYIYKY | |
| | TSYPDPILLMKSARSSCWSKDAEYGLYSIYQGGVF | |
| | ELKENDRIFVSVTNEHLIDMDHEASFFGAFLVGGG | |
| | GGSGGGGSGGGGSVRERGPQRVAAHITGTRGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKYTSYPDPILLMKSARSSCWSKDAEYG | |
| | LYSIYQGGVFELKENDRIFVSVTNEHLIDMDHEAS | |
| | FFGAFLVGGGGGSGGGGSGGGGSVRERGPQRVA | |
| | AHITGTRGRSNTLSSPNSKNEKALGRKINSWESSR | |
| | SGHSFLSNLHLRNGELVIHEKGFYYIYSQTYFRFQ | |
| | EEIKENTKNDKQMVQYIYKYTSYPDPILLMKSAR | |
| | SSCWSKDAEYGLYSIYQGGVFELKENDRIFVSVT | |
| | NEHLIDMDHEASFFGAFLVG | |
| Fc-T207 | DKTHTCPPCPAPELLGGPSVFLFPPKPKDTLMISR | 35 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTRGRSNTLSSPNSKNEK | |
| | ALGRKINSWESSRSGHSFLSNLHLRNGELVIHEKG | |
| | FYYIYSQTYFRFQEEIKENTKNDKQMVQYIYKYTS | |
| | YPDPILLMKSARSSCWSKDAEYGLYSIYQGGVFE | |
| | LKENDRIFVSVTNEHLIDMDHEASFFGAFLVGGG | |
| | GGSGGGGSGGGGSVRERGPQIVAAHITGTRGRSN | |
| | TLSSPNSKNEKALGRKINSWESSRSGHSFLSNLHL | |
| | RNGELVIHEKGFYYIYSQTYFRFQEEIKENTKNDK | |
| | QMVQYIYKYTSYPDPILLMKSARSSCWSKDAEYG | |
| | LYSIYQGGVFELKENDRIFVSVTNEHLIDMDHEAS | |
| | FFGAFLVGGGGGSGGGGSGGGGSVRERGPQIVAA | |
| | HITGTRGRSNTLSSPNSKNEKALGRKINSWESSRS | |
| | GHSFLSNLHLRNGELVIHEKGFYYIYSQTYFRFQE | |
| | EIKENTKNDKQMVQYIYKYTSYPDPILLMKSARSS | |
| | CWSKDAEYGLYSIYQGGVFELKENDRIFVSVTNE | |
| | HLIDMDHEASFFGAFLVG | |
| Fc-T208 | DKTHTCPPCPAPELLGGPSVFLFPPKPKDTLMISR | 36 |
| | TPEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTGGRSNTLSSPNSKNEK | |
| | ALGRKINSWESSRSGHSFLSNLHLRNGELVIHEKG | |
| | FYYIYSQTYFRFQEEIKENTKNDKQMVQYIYKYTS | |
| | YPDPILLMKSARNSCWSKDAEYGLYSIYQGGIFEL | |
| | KENDRIFVSVTNEHLIDMDHEASFFGAFLVGGGG | |
| | GSGGGGSGGGGSVRERGPQIVAAHITGTGGRSNT | |
| | LSSPNSKNEKALGRKINSWESSRSGHSFLSNLHLR | |
| | NGELVIHEKGFYYIYSQTYFRFQEEIKENTKNDKQ | |
| | MVQYIYKYTSYPDPILLMKSARNSCWSKDAEYGL | |
| | YSIYQGGIFELKENDRIFVSVTNEHLIDMDHEASFF | |
| | GAFLVGGGGGSGGGGSGGGGSVRERGPQIVAAHI | |
| | TGTGGRSNTLSSPNSKNEKALGRKINSWESSRSGH | |
| | SFLSNLHLRNGELVIHEKGFYYIYSQTYFRFQEEIK | |
| | ENTKNDKQMVQYIYKYTSYPDPILLMKSARNSC | |
| | WSKDAEYGLYSIYQGGIFELKENDRIFVSVTNEHL | |
| | IDMDHEASFFGAFLVG | |
| Fc-T209 | DKTHTCPPCPAPELLGGPSVFLFPPKPKDTLMISRT | 37 |
| | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTGGRSNTLSSPNSKNEK | |
| | ALGRKINSWESSRSGHSFLSNLHLRNGELVIHEKG | |
| | FYYIYSQTYFRFQEEIKENTKNDKQMVQYIYKYTS | |
| | YPDPILLMKSARSSCWSKDAEYGLYSIYQGGIFEL | |
| | KENDRIFVSVTNEHLIDMDHEASFFGAFLVGGGG | |
| | GSGGGGSGGGGSVRERGPQIVAAHITGTGGRSNT | |
| | LSSPNSKNEKALGRKINSWESSRSGHSFLSNLHLR | |
| | NGELVIHEKGFYYIYSQTYFRFQEEIKENTKNDKQ | |
| | MVQYIYKYTSYPDPILLMKSARSSCWSKDAEYGL | |
| | YSIYQGGIFELKENDRIFVSVTNEHLIDMDHEASFF | |
| | GAFLVGGGGGSGGGGSGGGGSVRERGPQIVAAHI | |
| | TGTGGRSNTLSSPNSKNEKALGRKINSWESSRSGH | |
| | SFLSNLHLRNGELVIHEKGFYYIYSQTYFRFQEEIK | |
| | ENTKNDKQMVQYIYKYTSYPDPILLMKSARSSCW | |
| | SKDAEYGLYSIYQGGIFELKENDRIFVSVTNEHLID | |
| | MDHEASFFGAFLVG | |
| Fc-T210 | DKTHTCPPCPAPELLGGPSVFLFPPKPKDTLMISRT | 38 |
| | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQIVAAHITGTRGRSNTLSSPNSKNEK | |
| | ALGRKINSWESSRSGHSFLSNLHLRNGELVIHEKG | |
| | FYYIYSQTYFRFQEEIKENTKNDKQMVQYIYKYTS | |
| | YPDPILLMKSARSSCWSKDAEYGLYSIYQGGIFEL | |
| | KENDRIFVSVTNEHLIDMDHEASFFGAFLVGGGG | |
| | GSGGGGSGGGGSVRERGPQIVAAHITGTRGRSNT | |
| | LSSPNSKNEKALGRKINSWESSRSGHSFLSNLHLR | |
| | NGELVIHEKGFYYIYSQTYFRFQEEIKENTKNDKQ | |
| | MVQYIYKYTSYPDPILLMKSARSSCWSKDAEYGL | |
| | YSIYQGGIFELKENDRIFVSVTNEHLIDMDHEASFF | |
| | GAFLVGGGGGSGGGGSGGGGSVRERGPQIVAAHI | |
| | TGTRGRSNTLSSPNSKNEKALGRKINSWESSRSGH | |
| | SFLSNLHLRNGELVIHEKGFYYIYSQTYFRFQEEIK | |
| | ENTKNDKQMVQYIYKYTSYPDPILLMKSARSSCW | |
| | SKDAEYGLYSIYQGGIFELKENDRIFVSVTNEHLID | |
| | MDHEASFFGAFLVG | |
| Fc-T211 | DKTHTCPPCPAPELLGGPSVFLFPPKPKDTLMISRT | 39 |
| | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGGGGGSGGGGSGG | |
| | GGSVRERGPQRVAAHITGTGGRSNTLSSPNSKNE | |
| | KALGRKINSWESSRSGHSFLSNLHLRNGELVIHEK | |
| | GFYYIYSQTYFRFQEEIKENTKNDKQMVQYIYKY | |
| | TSYPDPILLMKSARSSCWSKDAEYGLYSIYQGGIF | |
| | ELKENDRIFVSVTNEHLIDMDHEASFFGAFLVGGG | |
| | GGSGGGGSGGGGSVRERGPQRVAAHITGTGGRS | |
| | NTLSSPNSKNEKALGRKINSWESSRSGHSFLSNLH | |
| | LRNGELVIHEKGFYYIYSQTYFRFQEEIKENTKND | |
| | KQMVQYIYKYTSYPDPILLMKSARSSCWSKDAEY | |
| | GLYSIYQGGIFELKENDRIFVSVTNEHLIDMDHEA | |
| | SFFGAFLVGGGGGSGGGGSGGGGSVRERGPQRV | |
| | AAHITGTGGRSNTLSSPNSKNEKALGRKINSWESS | |
| | RSGHSFLSNLHLRNGELVIHEKGFYYIYSQTYFRF | |
| | QEEIKENTKNDKQMVQYIYKYTSYPDPILLMKSA | |
| | RSSCWSKDAEYGLYSIYQGGIFELKENDRIFVSVT | |
| | NEHLIDMDHEASFFGAFLVG | |
| G4S | GGGGS | 40 |
| Linker | | |
| siDR4- | GGAACUUUCCGGAAUGACAAUUCTG | 41 |
| Duplex-1 | | |
| siDR4- | GGACAAUGCUCACAACGAGAUUCTG | 42 |
| Duplex-2 | | |
| siDR4- | CAGAAUCUCGUUGUGAGCAUUGUCCUC | 43 |
| Duplex-3 | | |
| siDR5- | GUCACAUGACCGGUACUGGAAGAAA | 44 |
| Duplex-1 | | |
| siDR5- | UACCUUCUAGAUACAUGAACUUUCCAG | 45 |
| Duplex-2 | | |
| siDR5- | UCUGAGACAGUGCUUCGAUGACUTT | 46 |
| Duplex-3 | | |
| SiCIAP-1 | AAUGAUGCAUAACAAGCAGUGACACUA | 47 |
| Duplex-1 | | |
| SiCIAP-1 | AUACCUUUACAAGCGAGAGAACUGA | 48 |
| Duplex-2 | | |
| SiCIAP-1 | UUGGAAUAUACUUCAUAUUCUUAUCCA | 49 |
| Duplex-3 | | |
| 3 G₄S | GGGGSGGGGSGGGGS | 50 |
| domain | | |
| linker | | |

1. A method of treating a patient having colorectal cancer that has been determined to have high levels of DR4, the method comprising administering a therapeutically effective amount of a TRAIL-based therapeutic or a death receptor agonist.

2. A method of treating a patient having colorectal cancer that has been determined to have high levels of DR4 and low levels of cIAP1, the method comprising administering a therapeutically effective amount of a TRAIL-based therapeutic or a death receptor agonist.

3. The method of any one of the preceding claims, wherein the method comprises administration of a TRAIL-based therapeutic.

4. The method of any one of the preceding claims, wherein the TRAIL-based therapeutic is cross-linked.

5. The method of any one of the preceding claims, wherein the TRAIL-based therapeutic is PEGylated.

6. The method of any one of the preceding claims, wherein the TRAIL-based therapeutic is rhTRAIL.

7. The method of any one of the preceding claims, wherein the TRAIL-based therapeutic is a TRAIL polypeptide.

8. The method of claim 7, wherein the TRAIL polypeptide is Fc-TRAIL fusion polypeptide.

9. The method of claim 8, wherein the Fc-TRAIL fusion polypeptide is Fc-T191 (SEQ ID NO: 28).

10. The method of any one of the preceding claims, wherein the levels of DR4 are high enough and the levels of cIAP1 are low enough that the treatment results in greater than 50% cancer cell death.

11. The method of any one of claims 2-10, wherein the colorectal cancer has a DR4/cIAP1 ratio of at least about 0.5.

12. The method of claim 11, wherein the DR4/cIAP1 ratio is at least about 1.0.

13. The method of any one of the preceding claims, wherein the method comprises administration of a DR4 agonist.

14. The method of any one of the preceding claims, wherein the method comprises administration of a DRS agonist.

15. The method of any one of the preceding claims, wherein the method comprises administration of a dual DR4/DR5 agonist.

16. A method of treating a patient having a colorectal cancer that has been determined to have a DR4/cIAP1 ratio of at least about 0.5, wherein the method comprises administering a therapeutically effective amount of a Fc-TRAIL fusion polypeptide comprising SEQ ID NO:28.

17. A method of treating a patient having a colorectal cancer that has been determined to have a DR4/cIAP1 ratio of at least about 0.5, wherein the method comprises administering a therapeutically effective amount of a DR4, DRS, or DR4/DR5 agonist.

18. The method of claim 16 or 17, wherein the patient has a colorectal cancer that has been determined to have a DR4/cIAP1 ratio of at least about 0.7.

19. The method of claim 16 or 17, wherein the patient has a colorectal cancer that has been determined to have a DR4/cIAP1 ratio of at least about 1.0.


SEQUENCE LISTING (50 sequences)

SEQ ID NO: 1 - protein, 281 aa, Homo sapiens [sequence omitted]

SEQ ID NO: 2 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 3 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 4 - protein, 534 aa, Artificial Sequence
  Description of Artificial Sequence Synthetic polypeptide
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 5 - protein, 534 aa, Artificial Sequence
  Description of Artificial Sequence Synthetic polypeptide
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 6 - protein, 534 aa, Artificial Sequence
  Description of Artificial Sequence Synthetic polypeptide
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 7 - protein, 534 aa, Artificial Sequence
  Description of Artificial Sequence Synthetic polypeptide
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVG

SEQ ID NO: 8 - protein, 534 aa, Artificial Sequence
  Description of Artificial Sequence Synthetic polypeptide
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 9 - protein, 534 aa, Artificial Sequence
  Description of Artificial Sequence Synthetic polypeptide
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 10 - protein, 534 aa, Artificial Sequence
  Description of Artificial Sequence Synthetic polypeptide
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVG

SEQ ID NO: 11 - protein, 534 aa, Artificial Sequence
  Description of Artificial Sequence Synthetic polypeptide
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 12 - protein, 534 aa, Artificial Sequence
  Description of Artificial Sequence Synthetic polypeptide
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 13 - protein, 534 aa, Artificial Sequence
  Description of Artificial Sequence Synthetic polypeptide
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 14 - protein, 534 aa, Artificial Sequence; identical to SEQ ID NO: 4
  Description of Artificial Sequence Synthetic polypeptide

SEQ ID NO: 15 - protein, 534 aa, Artificial Sequence; identical to SEQ ID NO: 6
  Description of Artificial Sequence Synthetic polypeptide

SEQ ID NO: 16 - protein, 534 aa, Artificial Sequence; identical to SEQ ID NO: 5
  Description of Artificial Sequence Synthetic polypeptide

SEQ ID NO: 17 - protein, 534 aa, Artificial Sequence
  Description of Artificial Sequence Synthetic polypeptide
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 18 - protein, 534 aa, Artificial Sequence
  Description of Artificial Sequence Synthetic polypeptide
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 19 - protein, 534 aa, Artificial Sequence
  Description of Artificial Sequence Synthetic polypeptide
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 20 - protein, 534 aa, Artificial Sequence
  Description of Artificial Sequence Synthetic polypeptide
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 21 - protein, 534 aa, Artificial Sequence
  Description of Artificial Sequence Synthetic polypeptide
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 22 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 23 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 24 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 25 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVG

SEQ ID NO: 26 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 27 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 28 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVG

SEQ ID NO: 29 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 30 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 31 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 32 - protein, 775 aa, Artificial Sequence; identical to SEQ ID NO: 22
  Description of Artificial Sequence Synthetic polypeptide

SEQ ID NO: 33 - protein, 775 aa, Artificial Sequence; identical to SEQ ID NO: 24
  Description of Artificial Sequence Synthetic polypeptide

SEQ ID NO: 34 - protein, 775 aa, Artificial Sequence; identical to SEQ ID NO: 23
  Description of Artificial Sequence Synthetic polypeptide

SEQ ID NO: 35 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 36 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 37 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 38 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 39 - protein, 775 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 40 - protein, 5 aa, Artificial Sequence
  Description of Artificial Sequence Synthetic peptide
GGGGS

SEQ ID NO: 41 - DNA, 25 bp, Artificial Sequence
  Description of Artificial Sequence Synthetic oligonucleotide
  Description of Combined DNA/RNA Molecule Synthetic oligonucleotide
ggaacuuuccggaaugacaauuctg

SEQ ID NO: 42 - DNA, 25 bp, Artificial Sequence
  Description of Artificial Sequence Synthetic oligonucleotide
  Description of Combined DNA/RNA Molecule Synthetic oligonucleotide
ggacaaugcucacaacgagauuctg

SEQ ID NO: 43 - RNA, 27 nt, Artificial Sequence
  Description of Artificial Sequence Synthetic oligonucleotide
cagaaucucguugugagcauuguccuc

SEQ ID NO: 44 - RNA, 25 nt, Artificial Sequence
  Description of Artificial Sequence Synthetic oligonucleotide
gucacaugaccgguacuggaagaaa

SEQ ID NO: 45 - RNA, 27 nt, Artificial Sequence
  Description of Artificial Sequence Synthetic oligonucleotide
uaccuucuagauacaugaacuuuccag

SEQ ID NO: 46 - DNA, 25 bp, Artificial Sequence
  Description of Artificial Sequence Synthetic oligonucleotide
  Description of Combined DNA/RNA Molecule Synthetic oligonucleotide
ucugagacagugcuucgaugacutt

SEQ ID NO: 47 - RNA, 27 nt, Artificial Sequence
  Description of Artificial Sequence Synthetic oligonucleotide
aaugaugcauaacaagcagugacacua

SEQ ID NO: 48 - RNA, 25 nt, Artificial Sequence
  Description of Artificial Sequence Synthetic oligonucleotide
auaccuuuacaagcgagagaacuga

SEQ ID NO: 49 - RNA, 27 nt, Artificial Sequence
  Description of Artificial Sequence Synthetic oligonucleotide
uuggaauauacuucauauucuuaucca

SEQ ID NO: 50 - protein, 15 aa, Artificial Sequence
  Description of Artificial Sequence Synthetic peptide
GGGGSGGGGSGGGGS